(19) United States
(12) Patent Application Publication
(10) Pub. No.: US 2021/0363212 A1
(43) Pub. Date: Nov. 25, 2021

(54) GENETICALLY ENGINEERED CAR T CELLS THAT SECRET INTERLEUKIN-12 AND THERAPEUTIC USES THEREOF

(71) Applicant: CRISPR Therapeutics AG, Zug (CH)

(72) Inventors: Mohammed Ghonime, Cambridge, MA (US); Demetrios Kalaitzidis, Cambridge, MA (US); Jason Sagert, Cambridge, MA (US); Jonathan Alexander Terrett, Cambridge, MA (US)

(21) Appl. No.: 17/326,708

(22) Filed: May 21, 2021

Related U.S. Application Data
(60) Provisional application No. 63/140,996, filed on Jan. 25, 2021.
(60) Provisional application No. 63/028,190, filed on May 21, 2020.

(51) Int. Cl. C07K 14/54; C07K 14/725; C07K 14/74; C12N 15/90; C12N 15/11; C12N 9/22; C07K 16/28; C07K 14/705; C12N 5/0783; A61P 35/00; A61K 35/17; C12N 15/86

(52) U.S. Cl. CPC C07K 14/5434; A61K 2039/5156; C07K 14/70539; C12N 15/907; C12N 15/11; C12N 9/22; C07K 16/2875; C07K 14/70517; C07K 14/70578; C07K 14/70521; C12N 5/0636; A61P 35/00; A61K 35/17; C12N 15/86; C12N 2310/20; C12N 2800/80; C07K 2319/30; C07K 2319/33; C07K 2319/03; C07K 2319/02; C07K 2317/622; C07K 2317/76; C12N 2510/00; C07K 14/7051

(57) ABSTRACT

Genetically engineered immune cells such as T cells capable of secreting an interleukin-12 protein, for example, upon activation of the T cells. Such genetically engineered immune cells may further express a chimeric antigen receptor (CAR) targeting an antigen of interest, e.g., a tumor-associated antigen, a disrupted T cell receptor alpha chain constant (TRAC) gene, a disrupted beta-2-microglubulin (β2M) gene, a disrupted gene encoding the antigen of interest, or a combination thereof.

### CROSS REFERENCE TO RELATED APPLICATIONS

This application claims the benefit of the filing dates of U.S. Provisional Application No. 63/028,190, filed May 21, 2020 and U.S. Provisional Application No. 63/140,996, filed Jan. 25, 2021, the entire contents of each of which are incorporated by reference herein.

### SEQUENCE LISTING

The application contains a Sequence Listing that has been filed electronically in the form of a text file, created May 17, 2021, and named “095136-0329-015US1_SEQ.TXT” (171,048 bytes), the contents of which are incorporated by reference herein in their entirety

### BACKGROUND OF THE INVENTION

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these CAR T cells are injected into a patient, the receptors enable the T cells to kill cancer cells.

Currently, CAR T cell therapy showed limited efficacy in management of solid tumors. This may be caused by immunosuppressive cytokine and cellular tumor microenvironment, which suppresses adoptively transferred T cells. Improved CAR-T therapy is desired to enhance treatment efficacy for solid tumors.

### SUMMARY OF THE INVENTION

The present disclosure is based, at least in part, on the development of genetically engineered immune cells capable of secreting interleukin 12, for example, upon activation of the immune cells such as T cells. Such genetically engineered immune cells (e.g., T cells) are expected to be more effective in targeting tumor, for example, solid tumor, via, e.g., enhancing anti-tumor immunity.

Accordingly, one aspect of the present disclosure provides a population of genetically engineered immune cells, comprising immune cells that express an interleukin 12 (IL12) protein upon activation of the immune cells. The immune cells comprise an IL12 protein expression cassette, which comprises a transgene encoding the IL12 protein, a promoter in operable linkage to the transgene, and a binding site of a transcriptional regulatory factor associated with immune cell activation.

Exemplary binding sites include, but are not limited to, an NFκb binding site, an AP-1 binding site, a STAT5 binding site, a SMAD binding site, an NFAT binding site, or a combination thereof. In some instances, the binding site may comprise multiple copies of a binding motif of NFκb, AP-1, STAT5, SMAD, and/or NFAT.

The population of genetically engineered immune cells, in some instances, may further comprise a nucleic acid encoding a chimeric antigen receptor (CAR) specific to an antigen of interest. In some embodiments, the immune cells may further comprise a disrupted T cell receptor alpha chain constant (TRAC) gene, a disrupted beta-2-microglubulin (β2M) gene, a disrupted gene encoding the antigen of interest, or a combination thereof. In some examples, the antigen of interest is a tumor-associated antigen. In some examples, the tumor-associated antigen is a solid tumor antigen. In other examples, the tumor-associated antigen is a hematopoietic cancer antigen. In one specific example, the tumor associated antigen is CD70.

In another aspect, provided herein is a population of genetically engineered immune cells, comprising immune cells that, collectively, comprise an expression cassette of an interleukin 12 (IL12) protein, and a nucleic acid encoding a chimeric antigen receptor (CAR) specific to an antigen of interest. In some embodiments, the population of genetically engineered immune cells further comprises a disrupted TRAC gene, a disrupted β2M gene, a disrupted gene encoding the antigen of interest, or a combination thereof. “Collectively” as used here refers to the genetic edits exhibited by the population of cells as a whole. In some embodiments, at least a portion of the immune cells each comprise an expression cassette of an interleukin 12 (IL12) protein, a nucleic acid encoding a chimeric antigen receptor (CAR) specific to an antigen of interest, a disrupted TRAC gene, and a disrupted β2M gene. In some embodiments, at least a portion of the immune cells each comprise an expression cassette of an interleukin 12 (IL12) protein, a nucleic acid encoding a chimeric antigen receptor (CAR) specific to an antigen of interest, and a disrupted gene encoding the antigen of interest. In specific examples, at least a portion of the immune cells each comprise an expression cassette of an interleukin 12 (IL12) protein, a nucleic acid encoding a chimeric antigen receptor (CAR) specific to an antigen of interest, a disrupted TRAC gene, a disrupted β2M gene, and a disrupted gene encoding the antigen of interest.

In any of the genetically engineered immune cells disclosed herein, the nucleic acid encoding the CAR is inserted into a first genomic locus. For example, the nucleic acid encoding the CAR can be inserted into the first genomic locus by CRISPR/Cas-mediated gene editing and homologous recombination. In some instances, the CRISPR/Cas-mediated gene editing involves a first guide RNA targeting a site in the first genomic locus. The nucleic acid can be inserted at the site in the first genomic locus.

In some specific examples, the first genomic locus can be the TRAC gene and insertion of the nucleic acid encoding the CAR disrupts expression of the TRAC gene. For example, the first guide RNA used in the CRISPR/Cas-mediated gene editing system may target a TRAC site comprising the nucleotide sequence of 5′-AGAGCAACAGTGCTGTGGCC-3′ (SEQ ID NO: 22). The nucleic acid encoding the CAR can be inserted at the TRAC site comprising the nucleotide sequence of 5′-AGAGCAACAGTGCTGTGGCC-3′ (SEQ ID NO: 22). In one example, the disrupted TRAC gene has a deletion of a fragment comprising 5′-AGAGCAACAGTGCTGTGGCC-3′ (SEQ ID NO: 22), which can be replaced by the nucleic acid encoding the CAR.

In some embodiments, the expression cassette of the IL12 protein can be inserted into a second genomic locus (e.g., AAVS1, (32M, or the gene encoding the antigen of interest). In some examples, the expression cassette of the IL12 protein is inserted into the second genomic locus by CRISPR/Cas-mediated gene editing and homologous recombination. For example, the CRISPR/Cas-mediated gene editing may involve a second guide RNA targeting a site in the second genomic locus. The expression cassette of the IL12 protein can be inserted at site in the second genomic locus.

In some examples, the second genomic locus is AAVS1. The second guide RNA may target an AAVS1 site comprising the nucleotide sequence of 5′-GGGGCCACTAGGGACAGGAT-3′ (SEQ ID NO: 48). In some instances, the expression cassette of the IL12 protein can be inserted in the AAVS1 site comprising the nucleotide sequence of 5′-GGGGCCACTAGGGACAGGAT-3′ (SEQ ID NO: 48). In specific examples, the expression cassette of the IL12 protein may replace a fragment comprising the nucleotide sequence of 5′-GGGGCCACTAGGGACAGGAT-3′ (SEQ ID NO: 48) in the AAVS1 genomic locus.

In some examples, the second genomic locus is β2M. The second guide RNA may target a β2M site comprising the nucleotide sequence of 5′-GCTACTCTCTCTTTCTGGCC-3′ (SEQ ID NO: 36). For example, the expression cassette of the IL12 protein can be inserted in the β2M site comprising the nucleotide sequence of 5′-GCTACTCTCTCTTTCTGGCC-3′ (SEQ ID NO: 36). In specific examples, the expression cassette of the IL12 protein may replace a fragment comprising the nucleotide sequence of 5′-GCTACTCTCTCTTTCTGGCC-3′ (SEQ ID NO: 36) in the β2M gene.

In some examples, the second genomic locus is a gene encoding the antigen of interest, for example, a tumor-associated antigen, such as a solid tumor antigen or a hematopoietic cancer antigen. The IL12 can be inserted in the antigen of interest gene locus, thereby disrupting expression of the antigen of interest. In some examples, the antigen of interest is CD70. The second guide RNA may target a CD70 site comprising the nucleotide sequence of 5′-GCTTTGGTCCCATTGGTCGC-3′ (SEQ ID NO: 54). In some instances, the expression cassette of the IL12 protein can be inserted in the CD70 site comprising the nucleotide sequence of 5′-GCTTTGGTCCCATTGGTCGC-3′ (SEQ ID NO: 54). In specific examples, the expression cassette of the IL12 protein may replace a fragment comprising the nucleotide sequence of 5′-GCTTTGGTCCCATTGGTCGC-3′ (SEQ ID NO: 54) in the CD70 gene.

In any of the population of genetically engineered immune cells as disclosed herein, the CAR may bind a tumor-associated antigen, such as a solid tumor antigen or a hematopoietic cancer antigen. In one example, the CAR binds CD70. In some embodiments, the CAR binds CD70 and comprises an extracellular domain, a CD8 transmembrane domain, a 4-1BB co-stimulatory domain or a CD28 co-stimulatory domain (e.g., a 4-1BB co-stimulatory domain), and a CD3ζ cytoplasmic signaling domain, and wherein the extracellular domain is a single-chain antibody fragment (scFv) that binds CD70. In some examples, the scFv may comprise a heavy chain variable domain (V_{H}) comprising SEQ ID NO: 9, and a light chain variable domain (V_{L}) comprising SEQ ID NO: 10. In specific examples, the scFv comprises SEQ ID NO: 8. In specific examples, the CAR comprises SEQ ID NO: 19.

Any of the immune cells disclosed herein may be T cells, for example, human T cells. In some embodiments, the IL12 protein is a single chain polypeptide comprising IL12p40 and IL12p35. For example, the IL12 protein may comprise the amino acid sequence of SEQ ID NO:3. Alternatively, the IL12 protein may comprise the amino acid sequence of SEQ ID NO:

4.

In another aspect, the present disclosure provides a method for producing genetically engineered immune cells. In some embodiments, the method may comprise:

(i) introducing into a population of immune cells an expression cassette of an interleukin 12 (IL12) protein, wherein the expression cassette comprising a transgene encoding the IL12 protein, a promoter in operably linkage to the transgene, and a binding site of a transcriptional regulatory factor associated with immune cell activation (e.g., an NFκb binding site, an AP-1 binding site, a STAT5 binding site, a SMAD binding site, an NFAT binding site, or a combination thereof); and

(ii) harvesting the genetically engineered immune cells produced in step (i).

In some embodiments, step (ii) comprises purifying genetically engineered immune cells that express the IL12 protein upon activation of the immune cells. In some embodiments, the binding site comprises multiple copies of a binding motif of NFκb, AP-1, STAT5, SMAD, and/or NFAT.

In some examples, step (i) can be performed by delivering to the population of immune cells: (a) a first RNA-guided nuclease, (b) a first guide RNA (gRNA) targeting a genomic locus of interest (e.g., AAVS1, B2M, or CD70); and (c) a first vector comprising (1) the expression cassette of the IL12 protein, and (2) a first upstream and a first downstream nucleotide sequences flanking the expression cassette. The first upstream nucleotide sequence comprises a left region of homology to the first genomic locus of interest and the first downstream nucleotide sequence comprises a right region of homology to the first genomic locus of interest. In some examples, the population of immune cells in step (i) comprises a nucleic acid encoding a chimeric antigen receptor (CAR) specific to an antigen of interest. In other examples, immune cells in step (i) comprise, collectively, (1) a nucleic acid encoding a chimeric antigen receptor (CAR) specific to an antigen of interest, and (2) a disrupted TRAC gene, a disrupted β2M gene, and disrupted gene encoding the antigen of interest, or a combination thereof.

In some examples, the method disclosed herein may further comprise delivering to the immune cells: (d) a second RNA-guided nuclease, (e) a second guide RNA (gRNA) targeting a TRAC gene locus, and (f) a second vector comprising (1) a nucleic acid encoding a chimeric antigen receptor (CAR) specific to an antigen of interest, and (2) a second upstream and a second downstream nucleotide sequences flanking the nucleic acid encoding the CAR. The second upstream nucleotide sequence comprises a left region homology to the TRAC gene locus and the second downstream nucleotide sequence comprises a right region homology to the TRAC gene locus. In some examples, the method disclosed herein may further comprise delivering to the immune cells (g) a third guide RNA (gRNA) targeting a β2M gene locus, a fourth guide RNA (gRNA) targeting a gene encoding the antigen of interest, or a combination thereof.

In some examples, the components for the intended gene editing can be delivered to the immune cells simultaneously. In other examples, the components can be delivered to the immune cells sequentially, optionally via multiple electroporation. In some examples, the first vector, the second vector, and a ribonucleoprotein (RNP) comprising the RNA-guided nuclease(s) and the gRNAs can be delivered to the immune cells via one electroporation.

In other embodiments, a method for producing chimeric antigen receptor (CAR) T cells secreting an interleukin 12 protein may comprise:

(i) delivering to a population of immune cells
(a) a first vector comprising a nucleic acid encoding an interleukin 12 protein, and
(b) a second vector comprising a nucleic acid encoding a chimeric antigen receptor (CAR) specific to an antigen of interest; and

(ii) harvesting genetically engineered immune cells produced in step (i) expressing the CAR and the IL12 protein.

In some examples, the first vector comprises an expression cassette, which comprises the nucleic acid encoding the IL12 protein, a promoter in operable linkage to the nucleic acid, and a binding site of a transcriptional regulatory factor associated with immune cell activation, e.g., those disclosed herein.

In some examples, step (ii) comprises purifying genetically engineered immune cells expressing the CAR and the IL12 protein. In some examples, the method further comprises delivering to the population of immune cells: (a) one or more RNA-guided nucleases, and (b) a first guide RNA (gRNA) targeting a TRAC gene locus, a second gRNA targeting a β2M gene locus, a third gRNA targeting a genomic locus of interest, a fourth gRNA targeting a gene encoding the antigen of interest, or a combination thereof. In some examples, step (i) can be performed by delivering components (a)-(d) simultaneously. In other examples, step (i) is performed by delivering components (a)-(d) sequentially, optionally via multiple electroporation. In some examples, step (i) is performed by delivering the first vector, the second vector, and a ribonucleoprotein (RNP) comprising the RNA-guided nuclease(s) and the gRNAs via one electroporation.

In some instances the first vector further comprises a first upstream and a first downstream nucleotide sequences flanking the expression cassette. The first upstream nucleotide sequence comprises a left region of homology to the genomic locus of interest and the first downstream nucleotide sequence comprises a right region of homology to the genomic locus of interest. Alternatively or in addition, the second vector further comprises a second upstream and a second downstream nucleotide sequences flanking the nucleic acid encoding the CAR. The second upstream nucleotide sequence comprises a left region homology to the TRAC gene locus and the second downstream nucleotide sequence comprises a right region homology to the TRAC gene locus.

In any of the methods disclosed herein, the nucleic acid encoding the CAR is inserted into the TRAC gene, thereby disrupting expression of the TRAC gene. For example, the nucleic acid encoding the CAR can be inserted in a TRAC gene site comprising the nucleotide sequence of 5′-AGAGCAACAGTGCTGTGGCC-3′ (SEQ ID NO: 22). In some instances, the disrupted TRAC gene has a deletion of the nucleotide sequence of 5′-AGAGCAACAGTGCTGTGGCC-3′ (SEQ ID NO: 22). In specific examples, the nucleic acid encoding the CAR replaces a fragment comprising the nucleotide sequence of 5′-AGAGCAACAGTGCTGTGGCC-3′ (SEQ ID NO: 22). In some instances, the second gRNA targeting a TRAC site comprising the nucleotide sequence of 5′-AGAGCAACAGUGCUGUGGCC-3′ (SEQ ID NO: 25).

In any of the methods disclosed herein, the genomic locus of interest is AAVS1, β2M, or a gene encoding the antigen of interest. In some examples, the genomic locus of interest is β2M. The gRNA targeting a β2M site may comprise the nucleotide sequence of 5′-GCUACUCUCUCUUUCUGGCC-3′ (SEQ ID NO: 38). In some examples, the genomic locus of interest is AAVS1. The guide RNA targeting an AAVS1 site may comprise the nucleotide sequence of 5′-GGGGCCACUAGGGACAGGAU-3′ (SEQ ID NO: 50). In some examples, the genomic locus of interest is a gene encoding an antigen of interest, which may be a tumor-associated antigen, such as a solid tumor antigen or a hematopoietic cancer antigen. In one example, the tumor-associated antigen is CD70. In some examples, the gRNA targeting a CD70 site may comprise the nucleotide sequence of 5′-GCUUUGGUCCCAUUGGUCGC-3′ (SEQ ID NO: 56).

In any of the methods disclosed herein, the CAR binds a tumor-associated antigen, such as a solid tumor antigen or a hematopoietic cancer antigen. In some instances, the CAR is an anti-CD70 CAR as disclosed herein. In some embodiments, the immune cells used in any of the methods disclosed herein are human T cells. In some embodiments, the IL12 protein is a single chain polypeptide comprising IL12p40 and IL12p35, for example, SEQ ID NO: 3 or SEQ ID NO: 4.

In any of the methods disclosed herein, the first vector, the second vector, or both can be AAV vectors. In some embodiments, the first RNA-guided nuclease, the second RNA-guided nuclease, or both are the same enzyme, for example, a Cas9 enzyme.

In addition, the present disclosure provides a method for treating a solid tumor, the method comprising administering to a subject in need thereof an effective amount of any of the population of immune cells disclosed herein (e.g., T cells). In some examples, the subject is a human patient having a solid tumor such as renal cell carcinoma or lung cancer (e.g., non-small cell lung cancer).

In other embodiments, provided herein is a method for treating a hematopoietic malignancy such as a T cell or a B cell malignancy comprising administering to a subject in need thereof an effective amount of any of the population of immune cells disclosed herein (e.g., T cells). Examples include, but are not limited to, peripheral T cell lymphoma (PTCL), anaplastic large cell lymphoma (ALCL), Sezary syndrome (SS), non-smoldering acute adult T cell leukemia or lymphoma (ATLL), angioimmunoblastic T cell lymphoma (AITL), or diffuse large B cell lymphoma (DLBCL). In some embodiments, the subject has undergone a lymphodepletion treatment prior to administration of the population of immune cells. In some embodiments, the treatment method may further comprise subjecting the subject to a lymphodepleting treatment prior to administration of the population of immune cells.

In any of the methods disclosed herein, the population of immune cells may be allogeneic.

Also within the scope of the present disclosure are any of the populations of genetically engineered immune cells such as T cells disclosed herein for use in treating the solid tumor as also disclosed herein, or for manufacturing a medicament for use in the intended treatment.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic diagram depicting the general structure of an exemplary DNA template comprising an activation-dependent transgene.

FIG. 2 is a graph showing expression efficiency of the destabilized GFP (dsGFP) transgene in activation-dependent expression cassettes containing various transcriptional regulatory factor binding motifs in human T cells following treatment with either PMA/ionomycin (stimulated) or no treatment (resting).

FIG. 3 is a graph showing expression efficiency of the destabilized GFP (dsGFP) transgene in activation-dependent expression cassettes containing various transcriptional regulatory factor binding motifs in human anti-CD70 CAR T cells after co-culturing the CAR-T cells with A498 cells that express CD70.

FIGS. 4A and 4B are graphs showing secretion of IL12 over time from human CAR T cells comprising an IL12-expression cassette inserted at the AVVS1 locus after treatment with PMA/ionomycin (activation). FIG. 4A shows secretion of IL12 from CAR T cells that do not contain (mock) or contain constructs with the hIL12 902 slice variant as the transgene

(CTX1560 and CTX 1561). FIG. 4B shows secretion of IL12 from CAR T cells that do not contain (mock) or contain constructs with the hIL12 901 slice variant as the transgene (CTX1562 and CTX 1563).

FIGS. 5A and 5B are graphs showing secretion of IL12 over time from human anti-CD70 CAR T cells having an IL12-expression cassette inserted at the CD70 locus after treatment with PMA/ionomycin (activation). FIG. 5A shows secretion of IL12 from anti-CD70 CAR T cells that do not contain (mock) or contain constructs with the hIL12 902 slice variant as the transgene (CTX1569). FIG. 5B shows secretion of IL12 from anti-CD70 CAR T cells that do not contain (mock) or contain constructs with the hIL12 901 slice variant as the transgene (CTX1570 and CTX 1571).

FIGS. 6A and 6B are graphs showing secretion of IL12 over time from human anti-CD70 CAR T cells having an IL12-expression cassette inserted at the B2M locus after treatment with PMA/ionomycin (activation). FIG. 6A shows secretion of IL12 from anti-CD70 CAR T cells that do not contain (mock) or contain constructs with the hIL12 902 slice variant as the transgene (CTX1564 and CTX1565). FIG. 6B shows secretion of IL12 from anti-CD70 CAR T cells that do not contain (mock) or contain constructs with the hIL12 901 slice variant as the transgene (CTX1566 and CTX 1567).

FIG. 7 is a graph showing secretion of IL12 after treatment with PMA/ionomycin (activation) over time from human CAR T cells edited with constructs containing the hIL12 902 slice variant (CTX1565, CTX 1569, and CTX1561) or the hIL12 901 slice variant (CTX1563, CTX 1571, and CTX1563).

FIG. 8 is a graph showing secretion of IL12 24 hours after treatment with PMA/ionomycin or without treatment (no stimulation) from human anti-CD70 CAR T cells edited at one of three loci (AVVS1, CD70, or B2M) with constructs containing the either the hIL12 902 slice variant or the hIL12 901 slice variant.

FIGS. 9A and 9B are graphs showing secretion of IL12 from human anti-CD70 CAR T cells edited at one of three loci (AVVS1, CD70, or B2M) with constructs containing the either the hIL12 902 slice variant or the hIL12 901 slice variant as the transgene. FIG. 9A shows secretion of IL12 from edited CAR T cells 24 hours after co-culturing with A498 cells that express CD70 target antigen for the anti-CD70 CAR T (target cells) or cells that have not been co-cultured (no stimulation). FIG. 9B shows secretion of IL12 from edited CAR T cells 24 hours after co-culturing with 786-0 cells that express CD70 target antigen for the anti-CD70 CAR T (target cells) or cells that have not been co-cultured (no stimulation).

FIGS. 10A-10D are graphs showing that inducible IL12 enhanced anti-tumor effects of anti-CD70 CAR-T cells in multiple in vivo solid tumor mouse models. FIG. 10A: xenograft mice with H1975 cells, a non-small cell lung cancer model. FIG. 10B: xenograft mice with A498 cells, a renal cell carcinoma mouse model. FIG. 10C: xenograft mice with CAM-1 cells, a renal cell carcinoma mouse model. FIG. 10D: xenograft mice with BxCP3 cells, a pancreatic cancer mouse model.

### DETAILED DESCRIPTION OF THE INVENTION

The present disclosure is based, at least in part, on the development of genetically engineered immune cells such as T cells that have a knocked-in expression cassette for expressing an interleukin 12 (IL12) protein, which may be inserted at a genomic site of interest. Such genetically engineered immune cells are capable of secreting the IL12 protein, for example, upon activation. The genetically engineered immune cells may further express a chimeric antigen receptor (CAR) targeting an antigen of interest, for example, a tumor-associated antigen, such as a solid tumor antigen or a hematopoietic cancer antigen. Upon activation via engagement with the antigen of interest, the CAR-expressing immune cells (e.g., CAR-T cells) secrete the IL12 protein, which may help the host immune cells to be more active and thus more effectively kill disease cells (e.g., cancer cells) expressing the antigen of interest at a disease site. Accordingly, the genetically engineered immune cells disclosed herein are expected to be more effective in eliminating disease cells carrying the antigen of interest as relative to those that lack the IL12 knock-in. The design of conditional expressing IL12 upon T cell activation allows for secretion of the IL12 protein at a disease site (e.g., a tumor site), thereby minimizing impact on healthy tissues.

Accordingly, the present disclosure provides genetically engineered immune cells such as T cells carrying an exogenous IL12 expression cassette, which may conditionally express the encoded IL12 protein upon activation of the immune cells. Such genetically engineered immune cells may further express a chimeric antigen receptor targeting an antigen of interest (e.g., a solid tumor antigen or a tumor antigen associated with hematopoietic malignancies such as those disclosed herein). Alternatively or in addition, the genetically engineered immune cells may further have a disrupted T cell receptor alpha chain constant (TRAC) gene, a disrupted beta-2-microglubulin (β2M) gene, a disrupted gene encoding the antigen of interest, or a combination thereof. Also provided herein are uses of any of the genetically engineered immune cells for therapeutic purposes, e.g., allogeneic T cell therapy, and methods for producing such genetically engineered immune cells.

### I. Genetically Engineered Immune Cells

In one aspect, the present disclosure provides a genetically engineered immune cell (e.g., a T cell) or a population of genetically engineered immune cells (e.g., T cells) having a knocked-in expression cassette for producing an IL12 protein, and optionally one or more additional genetic edits as disclosed herein, e.g., expressing a CAR targeting an antigen of interest (e.g., a tumor-associated antigen, such as a solid tumor antigen or a hematopoietic cancer antigen, e.g., CD70), having a disrupted T cell receptor alpha chain constant (TRAC) gene, a disrupted beta-2-microglubulin (β2M) gene, a disrupted gene encoding the antigen of interest, or a combination thereof.

In some embodiments, the immune cells can be T cells. Parent T cells for making the genetically engineered T cells disclosed herein may be human T cells obtained from one or more healthy donors. Alternatively, the human T cells may be obtained from a patient such as a human cancer patient (e.g., who needs adoptive T cell therapy). In other examples, the T cells may be derived from a cultured T cell line, e.g., Jurkat, SupT1, etc. Primary T cells may be obtained from, e.g., blood, bone marrow, lymph node, the thymus, or other tissues or fluids. In some instances, the T cells can be enriched for or purified. For example, a subpopulation of T cells may be used for making the genetically engineered T cells disclosed herein. Examples include, but are not limited to, CD4⁺/CD8⁺ double positive T cells, CD4⁺ helper T cells (e.g., Th₁ and Th₂ cells), CD8⁺ T cells (e.g., cytotoxic T cells), tumor infiltrating cells (TILs), memory T cells, naïve T cells, or combinations thereof.

(A) Interleukin-12 (IL12) Knock-In

In some embodiments, the genetically engineered immune cells such as T cells disclosed herein have a knock-in of an expression cassette for producing an IL12 protein. As used herein, knock-in refers to introduction of a genetic material (e.g., exogenous) into a host via a genetic engineering process. The knocked-in genetic material may be inserted into a suitable genetic locus of the host cell. Alternatively, the knocked-in genetic material may exist extrachromosomal.

IL12 Proteins

Interleukin-12 is a disulfide-linked heterodimeric cytokine with multiple biological effects on the immune system. Naturally-occurring IL12 is a heterodimeric protein containing two subunits encoded by two separate genes, IL12A (coding for the p35 subunit, IL12p35) and IL12B (coding for the p40 subunit, IL12p40).

In some embodiments, the IL12 protein to be produced in the genetically engineered T cells disclosed herein may be a naturally-occurring IL12 protein or may comprise naturally-occurring IL12 subunits. A naturally-occurring IL12 protein or subunit may be from a suitable species, e.g., from a mammal such as mouse, rat, rabbit, pig, a non-human primate, or human. In some examples, the IL12 is a human protein. Exemplary human p35 and p40 subunits are provided as SEQ ID NOs: 1 and 2 herein. Naturally-occurring IL12 proteins from various species are well known in the art and their sequences can be retrieved from a public gene database such as GenBank. In some instances, the IL12 protein used herein may be a functional variant of a naturally-occurring IL12 (e.g., a functional variant of human IL12). Such a functional variant shares a high sequence homology (e.g., at least 85%, at least 90%, at least 95%, or above) with the wild-type counterpart and has substantially similar bioactivity as the wild-type counterpart (e.g., at least 80% of a bioactivity as compared with the wild-type counterpart).

In some embodiments, the IL12 protein to be produced in the genetically engineered immune cells such as T cells can be a heterodimer having the p35 and p40 subunits as separate polypeptide chains. In other embodiments, the IL12 protein may be a single polypeptide fusion protein comprising both the p35 and p40 subunits. In some instances, the two subunits can be linked directly. Alternatively, the two subunits can be linked via a peptide linker, for example, a G/S rich linker. In some embodiments, the peptide linker may be about one to about 20 amino acid residues. In some embodiments, the peptide linker may be about 15 amino acid residues. In some embodiments, the peptide linker is GGGGSGGGGSGGGGS (SEQ ID NO: 7).

In some examples, the p35 subunit is located at the N-terminal portion of the IL12 polypeptide (e.g., in a format of p35-linker-p40). In other examples, the p40 subunit is located at the N-terminal portion of the IL12 polypeptide (e.g., in a format of p40-linker-p35).

Table 1 below provides exemplary IL12 subunits and IL12 polypeptides for expressing in any of the genetically engineered immune cells such as T cells.

TABLE 1
Sequences of human IL12 (hIL12)
splice variants

| | SEQ ID | |
|---|---|---|
| Name | NO: | SEQUENCE |
| p35 | 1 | NLPVATPDPGMFPCLHHSQNLLRAVS |
| subunit | | NMLQKARQTLEFYPCTSEEIDHEDIT |
| | | KDKTSTVEACLPLELTKNESCLNSRE |
| | | TSFITNGSCLASRKTSFMMALCLSSI |
| | | YEDLKMYQVEFKTMNAKLLMDPKRQI |
| | | FLDQNMLAVIDELMQALNFNSETVPQ |
| | | KSSLEEPDFYKTKIKLCILLHAFRIR |
| | | AVTIDRVMSYLNAS |
| p40 | 2 | IWELKKDVYVVELDWYPDAPGEMVVL |
| subunit | | TCDTPEEDGITWTLDQSSEVLGSGKT |
| | | LTIQVKEFGDAGQYTCHKGGEVLSHS |
| | | LLLLHKKEDGIWSTDILKDQKEPKNK |
| | | TFLRCEAKNYSGRFTCWWLTTISTDL |
| | | TFSVKSSRGSSDPQGVTCGAATLSAE |
| | | RVRGDNKEYEYSVECQEDSACPAAEE |
| | | SLPIEVMVDAVHKLKYENYTSSFFIR |
| | | DIIKPDPPKNLQLKPLKNSRQVEVSW |
| | | EYPDTWSTPHSYFSLTFCVQVQGKSK |
| | | REKKDRVFTDKTSATVICRKNASISV |
| | | RAQDRYYSSSWSEWASVPCS |
| hIL12 * | 3 | MCPARSLLLVATLVLLDHLSLARNLP |
| (901) | | VATPDPGMFPCLHHSQNLLRAVSNML |
| | | QKARQTLEFYPCTSEEIDHEDITKDK |
| | | TSTVEACLPLELTKNESCLNSRETSF |
| | | ITNGSCLASRKTSFMMALCLSSIYED |
| | | LKMYQVEFKTMNAKLLMDPKRQIFLD |
| | | QNMLAVIDELMQALNFNSETVPQKSS |
| | | LEEPDFYKTKIKLCILLHAFRIRAVT |
| | | IDRVMSYLNASGGGGSGGGGSGGGGS |
| | | IWELKKDVYVVELDWYPDAPGEMVVL |
| | | TCDTPEEDGITWTLDQSSEVLGSGKT |
| | | LTIQVKEFGDAGQYTCHKGGEVLSHS |
| | | LLLLHKKEDGIWSTDILKDQKEPKNK |
| | | TFLRCEAKNYSGRFTCWWLTTISTDL |
| | | TFSVKSSRGSSDPQGVTCGAATLSAE |
| | | RVRGDNKEYEYSVECQEDSACPAAEE |
| | | SLPIEVMVDAVHKLKYENYTSSFFIR |
| | | DIIKPDPPKNLQLKPLKNSRQVEVSW |
| | | EYPDTWSTPHSYFSLTFCVQVQGKSK |
| | | REKKDRVFTDKTSATVICRKNASISV |
| | | RAQDRYYSSSWSEWASVPCS |
| hIL12* | 4 | MCHQQLVISWPSLVFLASPLVAIWEL |
| (902) | | KKDVYVVELDWYPDAPGEMVVLTCDT |
| | | PEEDGITWTLDQSSEVLGSGKTLTIQ |
| | | VKEFGDAGQYTCHKGGEVLSHSLLLL |
| | | HKKEDGIWSTDILKDQKEPKNKTFLR |
| | | CEAKNYSGRFTCWWLTTISTDLTFSV |
| | | KSSRGSSDPQGVTCGAATLSAERVRG |
| | | DNKEYEYSVECQEDSACPAAEESLPE |
| | | EVMVDAVHKLKYENYTSSFFIRDIIK |
| | | PDPPKNLQLKPLKNSRQVEVSWEYPD |
| | | TWSTPHSYFSLTFCVQVQGKSKREKK |
| | | DRVFTDKTSATVICRKNASISVRAQD |
| | | RYYSSSWSEWASVPCSGGGGSGGGGS |
| | | GGGGSRNLPVATPDPGMFPCLHHSQN |
| | | LLRAVSNMLQKARQTLEFYPCTSEEI |
| | | DHEDITKDKTSTVEACLPLELTKNES |
| | | CLNSRETSFITNGSCLASRKTSFMMA |
| | | LCLSSIYEDLKMYQVEFKTMNAKLLM |
| | | DPKRQIFLDQNMLAVIDELMQALNFN |
| | | SETVPQKSSLEEPDFYKTKIKLCILL |
| | | HAFRIRAVTIDRVMSYLNAS |
| Signal | 5 | MCPARSLLLVATLVLLDHLSLAR |
| peptide 1 | | |
| Signal | 6 | MCHQQLVISWFSLVFLASPLVA |
| peptide 2 | | |
| Peptide | 7 | GGGGSGGGGSGGGGS |
| Linker | | |

*linker underlined and signal peptide italicized.

An IL12 expression cassette refers to a nucleic acid molecule (e.g., a DNA molecule) comprising a nucleotide sequence encoding an IL12 protein and optionally further comprising a promoter, which can be in operably linkage to the IL12 coding sequence for control of IL12 expression in the host cells. In some embodiments expression cassettes may further comprise a 5′-untranslated region (5′-UTR sequence) (e.g., SEQ ID NO: 105) upstream a nucleotide sequence encoding an IL12 protein. In some embodiments, the IL12 expression cassette may comprise two coding regions, one encoding the p35 subunit and the other encoding the p40 subunit. Expression of the p35 and p40 subunits may be under the control of one promoter (in polycistronic format). Alternatively, expression of the p35 and p40 subunits may be under the control of distinct promoters. In other examples, two separate expression cassettes may be used, each for expressing one of the p35 and p40 subunits. The individual p35 and p40 polypeptides thus expressed may form a heterodimer in the host cell and be secreted. In other embodiments, the IL12 expression cassette may comprise one coding region encoding a polypeptide comprising the p35 subunit and the p40 subunit. Alternatively, an IL12 expression cassette may encompasses a transgene encoding a single polypeptide comprising both IL12p35 and IL12p40 subunits, e.g., those described herein.

Activation-Dependent Expression of IL12

An IL12 expression cassette disclosed herein may comprise a suitable promoter in operable linkage to the IL12 coding sequence(s) (transgene). The term “operably linked” means that the nucleotide sequence of interest is linked to regulatory sequence(s) in a manner that allows for expression of the encoded polypeptide. In some embodiments, the promoter may be a promoter (e.g., naturally-occurring or modified) of a gene expressed in immune cells such as in T cells. In some examples, the promoter can be a native IL2 promoter or a minimal IL2 promoter. In other examples, the promoter can be an ADE promoter, such as a late ADE promoter.

In some embodiments, the IL12 expression cassette may further comprise one or more regulatory elements that regulate IL12 expression in the host cells, for example, 5′ and/or 3′-UTRs, enhancers, silencers, polyA signaling sequences, or a combination thereof. The term “regulatory elements” is intended to include, for example, enhancers, silencers, transcriptional regulatory factor binding sites, and other expression control elements (e.g., polyadenylation signals). Such regulatory elements are well known in the art and are described, for example, in Goeddel; Gene Expression Technology: Methods in Enzymology 185, Academic Press, San Diego, Calif. (1990). Regulatory sequences include those that direct constitutive expression of a coding sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

In some examples, the IL12 expression cassette disclosed herein comprises a binding site of a transcriptional regulatory factor such as the expression of the IL12 protein can be triggered in specific tissues/cells or by specific cellular processes, for example, T cell activation. Transcriptional regulatory factors associated with immune cell activation include, but are not limited to, NF-κB, NFAT, AP1, STAT5 and SMAD. In some examples, the IL12 expression cassette may comprise one or more binding sites of one or more of such transcriptional regulatory factors. In some examples, a binding site of one transcriptional regulatory factor (e.g., NF-κB, NFAT, AP1, STAT5 and SMAD) may comprise multiple copies of the binding motifs for the transcriptional regulatory factor, which may be in tandem repeat in the binding site. In some examples, the binding site may be a hybrid binding site comprising at least one binding motif for one transcriptional regulatory factor and at least another binding motif for another transcriptional regulator factors. Binding sites of exemplary transcriptional regulatory factors are provided in Table 4 below.

In some examples, the IL12 expression cassette disclosed herein comprise a suitable promoter in operable linkage to the coding sequence of the IL12 protein and a binding site of APE The promoter may be an IL2 promoter such as a minimal IL2 prompter (e.g., SEQ ID NO:70). Alternatively, the promoter may be an ADE promoter such as a Late ADE promoter (e.g., SEQ ID NO:71). The AP1 binding site may comprise multiple copies of the AP1 binding motif, for example, 2x or 3x. In one specific example, the IL12 expression cassette disclosed herein comprise the minimal IL2 promoter in operable linkage to the coding sequence of an IL12 protein (e.g., hIL12(902)), and 3 copies of the AP1 binding motif.

Any of the IL12 expression cassettes disclosed herein, as well as the encoded IL12 polypeptides (e.g., SEQ ID NOs 3 and 4) are also within the scope of the present disclosure.

In specific examples, the genetically engineered immune cells disclosed herein express an interleukin 12 (IL12) protein upon activation of the immune cells. Such genetically engineered immune cells may comprise an expression cassette of the IL12 protein, which comprises a transgene encoding the IL12 protein (e.g., SEQ ID NO:3 or SEQ ID NO:4), a promoter operably linked to the transgene (e.g., a minimal IL2 promoter comprising the nucleotide sequence of SEQ ID NO: 70) and a binding site of a transcriptional regulatory factor associated with immune cell activation, for example one or more AP-1 binding sites (e.g., the 3x AP-1 binding site comprising the nucleotide sequence of SEQ ID NO: 67).

IL12 Expression Cassette Knock-In

Any of the IL12 expression cassettes disclosed herein can be introduced into immune cells via a conventional method or the gene editing methods disclosed herein. In some embodiments, the IL12 expression cassette may be knocked-in to the genome of the host cell at a genomic locus of interest. In some embodiments, the expression cassette of the IL12 protein can be knocked into a target genomic locus by replacing a portion of the target gene.

In some embodiments, the target genomic locus can be AAVS1. In some examples, the IL12 expression cassette may be inserted at or near a site of AAVS1 comprising the nucleotide sequence of 5′-GGGGCCACTAGGGACAGGAT-3′ (SEQ ID NO: 48). In specific examples, the IL12 expression cassette may replace a fragment comprising SEQ ID NO: 48 in the AAVS1 locus.

In other embodiments, the target genomic locus may be a (32M gene or a TRAC gene. In some examples, the IL12 expression cassette may be inserted at or near a site of the (32M gene comprising the nucleotide sequence of 5′-GCTTTGGTCCCATTGGTCGC-3′ (SEQ ID NO: 54). In specific examples, the IL12 expression cassette may replace a fragment comprising SEQ ID NO: 54 in the (32M gene locus.

In other embodiments, the IL12 expression cassette can be knocked into a gene encoding a target antigen, for example, the target antigen of a chimeric antigen receptor to be co-expressed in the genetically engineered immune cells such as T cells. Such target antigens may be tumor-associated antigens, e.g., a solid tumor antigen or a hematopoietic cancer antigen. One example is CD70. In some examples, the IL12 expression cassette may be inserted at or near a site in the CD70 gene comprising the nucleotide sequence of 5′-GCTTTGGTCCCATTGGTCGC-3′ (SEQ ID NO: 54). In specific examples, the IL12 expression cassette may replace a fragment comprising SEQ ID NO: 54.

(B) Chimeric Antigen Receptor (CAR) Knock-in

The genetically engineered immune cells such as T cells may be modified to knock-in a nucleic acid encoding a chimeric antigen receptor (CAR), for example, a CAR that targets a tumor antigen (e.g., CD70).

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3 zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

In some instances, a CAR can be a fusion polypeptide comprising an extracellular antigen binding domain that recognizes a target antigen (e.g., a single chain variable fragment (scFv) of an antibody or other antibody fragment) and an intracellular domain comprising a signaling domain of the T-cell receptor (TCR) complex (e.g., CD3c) and, in most cases, a co-stimulatory domain. (Enblad et al., Human Gene Therapy. 2015; 26(8):498-505). A CAR construct may further comprise a hinge and transmembrane domain between the extracellular domain and the intracellular domain. Examples of signal peptides include MLLLVTSLLLCELPHPAFLLIP (SEQ ID NO: 12) and MALPVTALLLPLALLLHAARP (SEQ ID NO: 13). Other signal peptides may be used.

(a) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen such as a tumor antigen. In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

Exemplary tumor-associated antigens include, but are not limited to, 5T4, CD2, CD3, CD7, CD5, CD19, CD20, CD22, CD30, CD38, CD70, CD123, CD133, CD171, CEA, CS1, BCMA, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, Claudin 18.2, and VEGFRII.

In some embodiments, the CAR constructs disclosed herein comprise a scFv extracellular domain capable of binding to CD70. In some examples, an anti-CD70 scFv may comprise a heavy chain variable domain (V_{H}) having the same heavy chain complementary determining regions (CDRs) as those in SEQ ID NO: 9 and/or a light chain variable domain (V_{L}) having the same light chain CDRs as those in SEQ ID NO: 10. See Table 2 below. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.bioinf.org.uk/abs/.

In other embodiments, an anti-CD70 scFv may be a functional variant of the exemplary anti-CD70 scFv provided in Table 2 below. Such a functional variant is substantially similar to the exemplary anti-CD70 scFv, both structurally and functionally. A functional variant comprises substantially the same V_{H} and V_{L} CDRs as the exemplary anti-CD70 scFv. For example, it may comprise only up to 8 (e.g., 8, 7, 6, 5, 4, 3, 2, or 1) amino acid residue variations in the total CDR regions relative to those in the exemplary anti-CD70 scFv and binds the same epitope of CD70 with substantially similar affinity (e.g., having a K_{D} value in the same order). In some instances, the functional variants may have the same heavy chain CDR3 as the exemplary anti-CD70 scFv, and optionally the same light chain CDR3 as the exemplary anti-CD70 scFv. Such an anti-CD70 scFv may comprise a V_{H} fragment having CDR amino acid residue variations (e.g., up to 5, for example, 5, 4, 3, 2, and 1) in only the heavy chain CDR1 and/or CDR2 as compared with the V_{H} of the exemplary anti-CD70 scFv. Alternatively or in addition, the anti-scFv antibody may further comprise a V_{L} fragment having CDR amino acid residue variations (e.g., up to 5, for example, 5, 4, 3, 2, and 1) in only the light chain CDR1 and/or CDR2 as compared with the V_{L} of the exemplary anti-CD70 scFv. In some examples, the amino acid residue variations can be conservative amino acid residue substitutions.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-CD70 scFv disclosed herein may be in the format of, from N-terminus to C-terminus, V_{H}-linker-V_{L}. In some examples, the anti-CD70 scFv comprises a V_{H} fragment of SEQ ID NO: 9 and a VL fragment of SEQ ID NO: 10. Specific examples of anti-CD70 scFv are provided in Table 2 below.

(b) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of FVPVFLPAKPTTTPAPRPPTPAPTIAS QPLSLRPEACRPAAGG AVHTRGLDFACDIYIWAPLAGTCGVLLLSLVITLYCNHRNR (SEQ ID NO: 14) or IYIWAPLAGTCGVLLLSLVITLY (SEQ ID NO: 15). Other transmembrane domains may be used.

(c) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

(d) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

It should be understood that methods described herein encompasses more than one suitable CAR that can be used to produce genetically engineered T cells expressing the CAR, for example, those known in the art or disclosed herein. Examples can be found in, e.g., WO 2019/097305A2, and WO2019215500, the relevant disclosures of each of the prior applications are incorporated by reference herein for the purpose and subject matter referenced herein.

Amino acid sequences of the components of exemplary anti-CD70 CARs are provided in Table 2 below.

TABLE 2
Sequences of Exemplary Anti-CD70
CAR Construct Components.

| | | SEQ |
|---|---|---|
| | | ID |
| Description | Sequence | NO: |
| CD70B | QVQLVQSGAEVKKPGASVKVSC | 8 |
| scFv | KASGYTFTNYGMNWVRQAPGQG | |
| amino | LKWMGWINTYTGEPTYADAFKG | |
| acid | RVTMTRDTSISTAYMELSRLRS | |
| sequence | DDTAVYYCARDYGDYGMDYWGQ | |
| (linker | GTTVTVSSGGGGSGGGGSGGGG | |
| underlined) | SGDIVMTQSPDSLAVSLGERAT | |
| | INCRASKSVSTSGYSFMHWYQQ | |
| | KPGQPPKLLIYLASNLESGVPD | |
| | RFSGSGSGTDFTLTISSLQAED | |
| | VAVYYCQHSREVPWTFGQGTKV | |
| | EIK | |
| CD70 VH | QVQLVQSGAEVKKPGASVKVSC | 9 |
| | KASGYTFTNYGMNWVRQAPGQG | |
| | LKWMGWINTYTGEPTYADAFKG | |
| | RVTMTRDTSISTAYMELSRLRS | |
| | DDTAVYYCARDYGDYGMDYWGQ | |
| | GTTVTVSS | |
| CD70 VL | DIVMTQSPDSLAVSLGERATIN | 10 |
| | CRASKSVSTSGYSFMHWYQQKP | |
| | GQPPKLLIYLASNLESGVPDRF | |
| | SGSGSGTDFTLTISSLQAEDVA | |
| | VYYCQHSREVPWTFGQGTKVEI | |
| | K | |
| Linker | GGGGSGGGGSGGGGSG | 11 |
| signal | MLLLVTSLLLCELPHPAFLLI | 12 |
| peptide | P | |
| signal | MALPVTALLLPLALLLHAARP | 13 |
| peptide | | |
| CD8a | FVPVFLPAKPTTTPAPRPPTPA | 14 |
| trans- | PTIASQPLSLRPEACRPAAGGA | |
| membrane | VHTRGLDFACDIYIWAPLAGTC | |
| domain | GVLLLSLVITLYCNHRNR | |
| CD8a | IYIWAPLAGTCGVLLLSLVITL | 15 |
| trans- | Y | |
| membrane | | |
| 4-1BB | KRGRKKLLYIFKQPFMRPVQTT | 16 |
| amino | QEEDGCSCRFPEEEEGGCEL | |
| acid | | |
| sequence | | |
| CD28 | SKRSRLLHSDYMNMTPRRPGPT | 17 |
| amino | RKHYQPYAPPRDFAAYRS | |
| acid | | |
| sequence | | |
| CD3ζ amino | RVKFSRSADAPAYQQGQNQLYN | 18 |
| acid | ELNLGRREEYDVLDKRRGRDPE | |
| sequence | MGGKPRRKNPQEGLYNELQKDK | |
| | MAEAYSEIGMKGERRRGKGHDG | |
| | LYQGLSTATKDTYDALHMQALP | |
| | PR | |
| CD70 CAR | MALPVTALLLPLALLLHAARP | 19 |
| amino | QVQLVQSGAEVKKPGASVKVS | |
| acid | CKASGYTFTNYGMNWVRQAPG | |
| sequence | QGLKWMGWINTYTGEPTYADA | |
| (CD70B | FKGRVTMTRDTSISTAYMELS | |
| scFV | RLRSDDTAVYYCARDYGDYGM | |
| with 41BB) | DYWGQGTTVTVSSGGGGSGGG | |
| | GSGGGGSGDIVMTQSPDSLAV | |
| | SLGERATINCRASKSVSTSGY | |
| | SFMHWYQQKPGQPPKLLIYLA | |
| | SNLESGVPDRFSGSGSGTDFT | |
| | LTISSLQAEDVAVYYCQHSRE | |
| | VPWTFGQGTKVEIKSAAAFVP | |
| | VFLPAKPTTTPAPRPPTPAPT | |
| | IASQPLSLRPEACRPAAGGAV | |
| | HTRGLDFACDIYIWAPLAGTC | |
| | GVLLLSLVITLYCNHRNRKRG | |
| | RKKLLYIFKQPFMRPVQTTQE | |
| | EDGCSCRFPEEEEGGCELRVK | |
| | FSRSADAPAYQQGQNQLYNEL | |
| | NLGRREEYDVLDKRRGRDPEM | |
| | GGKPRRKNPQEGLYNELQKDK | |
| | MAEAYSEIGMKGERRRGKGHD | |
| | GLYQGLSTATKDTYDALHMQA | |
| | LPPR | |
| EFIα | GGCTCCGGTGCCCGTCAGTGG | 20 |
| promoter | GCAGAGCGCACATCGCCCACA | |
| | GTCCCCGAGAAGTTGGGGGGA | |
| | GGGGTCGGCAATTGAACCGGT | |
| | GCCTAGAGAAGGTGGCGCGGG | |
| | GTAAACTGGGAAAGTGATGTC | |
| | GTGTACTGGCTCCGCCTTTTT | |
| | CCCGAGGGTGGGGGAGAACCG | |
| | TATATAAGTGCAGTAGTCGCC | |
| | GTGAACGTTCTTTTTCGCAAC | |
| | GGGTTTGCCGCCAGAACACAG | |
| | GTAAGTGCCGTGTGTGGTTCC | |
| | CGCGGGCCTGGCCTCTTTACG | |
| | GGTTATGGCCCTTGCGTGCCT | |
| | TGAATTACTTCCACTGGCTGC | |
| | AGTACGTGATTCTTGATCCCG | |
| | AGCTTCGGGTTGGAAGTGGGT | |
| | GGGAGAGTTCGAGGCCTTGCG | |
| | CTTAAGGAGCCCCTTCGCCTC | |
| | GTGCTTGAGTTGAGGCCTGGC | |
| | CTGGGCGCTGGGGCCGCCGCG | |
| | TGCGAATCTGGTGGCACCTTC | |
| | GCGCCTGTCTCGCTGCTTTCG | |
| | ATAAGTCTCTAGCCATTTAAA | |
| | ATTTTTGATGACCTGCTGCGA | |
| | CGCTTTTTTTCTGGCAAGATA | |
| | GTCTTGTAAATGCGGGCCAAG | |
| | ATCTGCACACTGGTATTTCGG | |
| | TTTTTGGGGCCGCGGGCGGCG | |
| | ACGGGGCCCGTGCGTCCCAGC | |
| | GCACATGTTCGGCGAGGCGGG | |
| | GCCTGCGAGCGCGGCCACCGA | |
| | GAATCGGACGGGGGTAGTCTC | |
| | AAGCTGGCCGGCCTGCTCTGG | |
| | TGCCTGGCCTCGCGCCGCCGT | |
| | GTATCGCCCCGCCCTGGGCGG | |
| | CAAGGCTGGCCCGGTCGGCAC | |
| | CAGTTGCGTGAGCGGAAAGAT | |
| | GGCCGCTTCCCGGCCCTGCTG | |
| | CAGGGAGCTCAAAATGGAGGA | |
| | CGCGGCGCTCGGGAGAGCGGG | |
| | CGGGTGAGTCACCCACACAAA | |
| | GGAAAAGGGCCTTTCCGTCCT | |
| | CAGCCGTCGCTTCATGTGACT | |
| | CCACGGAGTACCGGGCGCCGT | |
| | CCAGGCACCTCGATTAGTTCT | |
| | CGAGCTTTTGGAGTACGTCGT | |
| | CTTTAGGTTGGGGGGAGGGGT | |
| | TTTATGCGATGGAGTTTCCCC | |
| | ACACTGAGTGGGTGGAGACTG | |
| | AAGTTAGGCCAGCTTGGCACT | |
| | TGATGTAATTCTCCTTGGAAT | |
| | TTGCCCTTTTTGAGTTTGGAT | |
| | CTTGGTTCATTCTCAAGCCTC | |
| | AGACAGTGGTTCAAAGTTTTT | |
| | TTCTTCCATTTCAGGTGTCGT | |
| | GA | |
| CD70 LHA | GAGATGTAAGGAGCTGCTGTG | 21 |
| to RHA | ACTTGCTCAAGGCCTTATATC | |
| (CD70B | GAGTAAACGGTAGTGCTGGGG | |
| scFV | CTTAGACGCAGGTGTTCTGAT | |
| with | TTATAGTTCAAAACCTCTATC | |
| 41BB) | AATGAGAGAGCAATCTCCTGG | |
| | TAATGTGATAGATTTCCCAAC | |
| | TTAATGCCAACATACCATAAA | |
| | CCTCCCATTCTGCTAATGCCC | |
| | AGCCTAAGTTGGGGAGACCAC | |
| | TCCAGATTCCAAGATGTACAG | |
| | TTTGCTTTGCTGGGCCTTTTT | |
| | CCCATGCCTGCCTTTACTCTG | |
| | CCAGAGTTATATTGCTGGGGT | |
| | TTTGAAGAAGATCCTATTAAA | |
| | TAAAAGAATAAGCAGTATTAT | |
| | TAAGTAGCCCTGCATTTCAGG | |
| | TTTCCTTGAGTGGCAGGCCAG | |
| | GCCTGGCCGTGAACGTTCACT | |
| | GAAATCATGGCCTCTTGGCCA | |
| | AGATTGATAGCTTGTGCCTGT | |
| | CCCTGAGTCCCAGTCCATCAC | |
| | GAGCAGCTGGTTTCTAAGATG | |
| | CTATTTCCCGTATAAAGCATG | |
| | AGACCGTGACTTGCCAGCCCC | |
| | ACAGAGCCCCGCCCTTGTCCA | |
| | TCACTGGCATCTGGACTCCAG | |
| | CCTGGGTTGGGGCAAAGAGGG | |
| | AAATGAGATCATGTCCTAACC | |
| | CTGATCCTCTTGTCCCACAGA | |
| | TATCCAGAACCCTGACCCTGC | |
| | CGTGTACCAGCTGAGAGACTC | |
| | TAAATCCAGTGACAAGTCTGT | |
| | CTGCCTATTCACCGATTTTGA | |
| | TTCTCAAACAAATGTGTCACA | |
| | AAGTAAGGATTCTGATGTGTA | |
| | TATCACAGACAAAACTGTGCT | |
| | AGACATGAGGTCTATGGACTT | |
| | CAGGCTCCGGTGCCCGTCAGT | |
| | GGGCAGAGCGCACATCGCCCA | |
| | CAGTCCCCGAGAAGTTGGGGG | |
| | GAGGGGTCGGCAATTGAACCG | |
| | GTGCCTAGAGAAGGTGGCGCG | |
| | GGGTAAACTGGGAAAGTGATG | |
| | TCGTGTACTGGCTCCGCCTTT | |
| | TTCCCGAGGGTGGGGGAGAAC | |
| | CGTATATAAGTGCAGTAGTCG | |
| | CCGTGAACGTTCTTTTTCGCA | |
| | ACGGGTTTGCCGCCAGAACACA | |
| | GGTAAGTGCCGTGTGTGGTTCC | |
| | CGCGGGCCTGGCCTCTTTACGG | |
| | GTTATGGCCCTTGCGTGCCTTG | |
| | AATTACTTCCACTGGCTGCAGT | |
| | ACGTGATTCTTGATCCCGAGCT | |
| | TCGGGTTGGAAGTGGGTGGGAG | |
| | AGTTCGAGGCCTTGCGCTTAAG | |
| | GAGCCCCTTCGCCTCGTGCTTG | |
| | AGTTGAGGCCTGGCCTGGGCGC | |
| | TGGGGCCGCCGCGTGCGAATCT | |
| | GGTGGCACCTTCGCGCCTGTCT | |
| | CGCTGCTTTCGATAAGTCTCTA | |
| | GCCATTTAAAATTTTTGATGAC | |
| | CTGCTGCGACGCTTTTTTTCTG | |
| | GCAAGATAGTCTTGTAAATGCG | |
| | GGCCAAGATCTGCACACTGGTA | |
| | TTTCGGTTTTTGGGGCCGCGGG | |
| | CGGCGACGGGGCCCGTGCGTCC | |
| | CAGCGCACATGTTCGGCGAGGC | |
| | GGGGCCTGCGAGCGCGGCCACC | |
| | GAGAATCGGACGGGGGTAGTCT | |
| | CAAGCTGGCCGGCCTGCTCTGG | |
| | TGCCTGGCCTCGCGCCGCCGTG | |
| | TATCGCCCCGCCCTGGGCGGCA | |
| | AGGCTGGCCCGGTCGGCACCAG | |
| | TTGCGTGAGCGGAAAGATGGCC | |
| | GCTTCCCGGCCCTGCTGCAGGG | |
| | AGCTCAAAATGGAGGACGCGGC | |
| | GCTCGGGAGAGCGGGCGGGTGA | |
| | GTCACCCACACAAAGGAAAAGG | |
| | GCCTTTCCGTCCTCAGCCGTCG | |
| | CTTCATGTGACTCCACGGAGTA | |
| | CCGGGCGCCGTCCAGGCACCTC | |
| | GATTAGTTCTCGAGCTTTTGGA | |
| | GTACGTCGTCTTTAGGTTGGGG | |
| | GGAGGGGTTTTATGCGATGGAG | |
| | TTTCCCCACACTGAGTGGGTGG | |
| | AGACTGAAGTTAGGCCAGCTTG | |
| | GCACTTGATGTAATTCTCCTTG | |
| | GAATTTGCCCTTTTTGAGTTTG | |
| | GATCTTGGTTCATTCTCAAGCC | |
| | TCAGACAGTGGTTCAAAGTTTT | |
| | TTTCTTCCATTTCAGGTGTCGT | |
| | GACCACCATGGCGCTTCCGGTG | |
| | ACAGCACTGCTCCTCCCCTTGG | |
| | CGCTGTTGCTCCACGCAGCAAG | |
| | GCCGCAGGTCCAGTTGGTGCAA | |
| | AGCGGGGCGGAGGTGAAAAAAC | |
| | CCGGCGCTTCCGTGAAGGTGTC | |
| | CTGTAAGGCGTCCGGTTATACG | |
| | TTCACGAACTACGGGATGAATT | |
| | GGGTTCGCCAAGCGCCGGGGCA | |
| | GGGACTGAAATGGATGGGGTGG | |
| | ATAAATACCTACACCGGCGAAC | |
| | CTACATACGCCGACGCTTTTAA | |
| | AGGGCGAGTCACTATGACGCGC | |
| | GATACCAGCATATCCACCGCAT | |
| | ACATGGAGCTGTCCCGACTCCG | |
| | GTCAGACGACACGGCTGTCTAC | |
| | TATTGTGCTCGGGACTATGGCG | |
| | ATTATGGCATGGACTACTGGGG | |
| | TCAGGGTACGACTGTAACAGTT | |
| | AGTAGTGGTGGAGGCGGCAGTG | |
| | GCGGGGGGGGAAGCGGAGGAGG | |
| | GGGTTCTGGTGACATAGTTATG | |
| | ACCCAATCCCCAGATAGTTTGG | |
| | CGGTTTCTCTGGGCGAGAGGGC | |
| | AACGATTAATTGTCGCGCATCA | |
| | AAGAGCGTTTCAACGAGCGGAT | |
| | ATTCTTTTATGCATTGGTACCA | |
| | GCAAAAACCCGGACAACCGCCG | |
| | AAGCTGCTGATCTACTTGGCTT | |
| | CAAATCTTGAGTCTGGGGTGCC | |
| | GGACCGATTTTCTGGTAGTGGA | |
| | AGCGGAACTGACTTTACGCTCA | |
| | CGATCAGTTCACTGCAGGCTGA | |
| | GGATGTAGCGGTCTATTATTGC | |
| | CAGCACAGTAGAGAAGTCCCCT | |
| | GGACCTTCGGTCAAGGCACGAA | |
| | AGTAGAAATTAAAAGTGCTGCT | |
| | GCCTTTGTCCCGGTATTTCTCC | |
| | CAGCCAAACCGACCACGACTCC | |
| | CGCCCCGCGCCCTCCGACACCC | |
| | GCTCCCACCATCGCCTCTCAAC | |
| | CTCTTAGTCTTCGCCCCGAGGC | |
| | ATGCCGACCCGCCGCCGGGGGT | |
| | GCTGTTCATACGAGGGGCTTGG | |
| | ACTTCGCTTGTGATATTTACAT | |
| | TTGGGCTCCGTTGGCGGGTACG | |
| | TGCGGCGTCCTTTTGTTGTCAC | |
| | TCGTTATTACTTTGTATTGTAA | |
| | TCACAGGAATCGCAAACGGGGC | |
| | AGAAAGAAACTCCTGTATATAT | |
| | TCAAACAACCATTTATGAGACC | |
| | AGTACAAACTACTCAAGAGGAA | |
| | GATGGCTGTAGCTGCCGATTTC | |
| | CAGAAGAAGAAGAAGGAGGATG | |
| | TGAACTGCGAGTGAAGTTTTCC | |
| | CGAAGCGCAGACGCTCCGGCAT | |
| | ATCAGCAAGGACAGAATCAGCT | |
| | GTATAACGAACTGAATTTGGGA | |
| | CGCCGCGAGGAGTATGACGTGC | |
| | TTGATAAACGCCGGGGGAGAGA | |
| | CCCGGAAATGGGGGGTAAACCC | |
| | CGAAGAAAGAATCCCCAAGAAG | |
| | GACTCTACAATGAACTCCAGAA | |
| | GGATAAGATGGCGGAGGCCTAC | |
| | TCAGAAATAGGTATGAAGGGCG | |
| | AACGACGACGGGGAAAAGGTCA | |
| | CGATGGCCTCTACCAAGGGTTG | |
| | AGTACGGCAACCAAAGATACGT | |
| | ACGATGCACTGCATATGCAGGC | |
| | CCTGCCTCCCAGATAATAATAA | |
| | AATCGCTATCCATCGAAGATGG | |
| | ATGTGTGTTGGTTTTTTGTGTG | |
| | TGGAGCAACAAATCTGACTTTG | |
| | CATGTGCAAACGCCTTCAACAA | |
| | CAGCATTATTCCAGAAGACACC | |
| | TTCTTCCCCAGCCCAGGTAAGG | |
| | GCAGCTTTGGTGCCTTCGCAGG | |
| | CTGTTTCCTTGCTTCAGGAATG | |
| | GCCAGGTTCTGCCCAGAGCTCT | |
| | GGTCAATGATGTCTAAAACTCC | |
| | TCTGATTGGTGGTCTCGGCCTT | |
| | ATCCATTGCCACCAAAACCCTC | |
| | TTTTTACTAAGAAACAGTGAGC | |
| | CTTGTTCTGGCAGTCCAGAGAA | |
| | TGACACGGGAAAAAAGCAGATG | |
| | AAGAGAAGGTGGCAGGAGAGGG | |
| | CACGTGGCCCAGCCTCAGTCTC | |
| | TCCAACTGAGTTCCTGCCTGCC | |
| | TGCCTTTGCTCAGACTGTTTGC | |
| | CCCTTACTGCTCTTCTAGGCCT | |
| | CATTCTAAGCCCCTTCTCCAAG | |
| | TTGCCTCTCCTTATTTCTCCCT | |
| | GTCTGCCAAAAAATCTTTCCCA | |
| | GCTCACTAAGTCAGTCTCACGC | |
| | AGTCACTCATTAACCCACCAAT | |
| | CACTGATTGTGCCGGCACATGA | |
| | ATGCACCAGGTGTTGAAGTGGA | |
| | GGAATTAAAAAGTCAGATGAGG | |
| | GGTGTGCCCAGAGGAAGCACCA | |
| | TTCTAGTTGGGGGAGCCCATCT | |
| | GTCAGCTGGGAAAAGTCCAAAT | |
| | AACTTCAGATTGGAATGTGTTT | |
| | TAACTCAGGGTTGAGAAAACAG | |
| | CTACCTTCAGGACAAAAGTCAG | |
| | GGAAGGGCTCTCTGAAGAAATG | |
| | CTACTTGAAGATACCAGCCCTA | |
| | CCAAGGGCAGGGAGAGGACCCT | |
| | ATAGAGGCCTGGGACAGGAGCT | |
| | CAATGAGAAAGG | |

In some embodiments, any of the CAR-coding nucleic acids disclosed herein may be inserted in a TRAC gene locus as disclosed herein, for example, replacing a fragment comprising the nucleotide sequence of SEQ ID NO: 22.

(C) Disruption of Endogenous Genes

In addition to the knock-in of any of the IL12 expression cassettes disclosed herein, the chimeric antigen receptor-encoding nucleic acids, or both, the genetically engineered immune cells such as T cells disclosed herein may further comprise one or more disrupted endogenous genes via gene editing. Examples include TRAC, B2M, and the gene encoding the target antigen of the CAR (e.g., the CD70 gene).

As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the all or part of the deleted fragment may be located within the gene region targeted by a designed guide RNA such as those disclosed herein (known as on-target sequence). In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region. In other embodiments, a disrupted gene may be described as having a mutation of a fragment located within the gene region targeted by a designed guide RNA such as those disclosed herein.

In some instances, the disrupted TRAC gene in the genetically engineered T cells disclosed herein may comprise a deletion, for example, a deletion of a fragment in Exon 1 of the TRAC gene locus. In some examples, the disrupted TRAC gene comprises a deletion of a fragment comprising the nucleotide sequence of SEQ ID NO: 22, which is the target site of TRAC guide RNA TA-1. See Table 3 below. In some examples, the fragment of SEQ ID NO: 22 may be replaced by a nucleic acid encoding any of the CAR, for example, the anti-CD70 CAR disclosed herein.

In some instances, the disrupted B2M gene in the genetically engineered T cells disclosed herein may be generated using the CRISPR/Cas technology. In some examples, a B2M gRNA provided in Table 3 may be used. The disrupted B2M gene may comprise a nucleotide sequence of any one of SEQ ID NOs: 42-47. In some examples, the gene editing approach can be used in combination with homologous recombination such that the IL12 expression cassette can be inserted at or near the target antigen gene (e.g., the B2M gene) thereby disrupting expression of the target antigen. See descriptions above.

In some instances, the gene of the target antigen (e.g., CD70) may be disrupted, for example, using the CRISPR/Cas technology. In some examples, a CD70 gRNA provided in Table 3 may be used. In some examples, the gene editing approach can be used in combination with homologous recombination such that the IL12 expression cassette can be inserted into the target antigen gene (e.g., the CD70 gene) thereby disrupting expression of the target antigen. See descriptions above.

(D) Exemplary Genetically Engineered CAR T Cells

In some embodiments, provided herein is a population of genetically engineered immune cells (e.g., T cells such as human T cells), which collectively (i.e., in the whole cell population) express any of the IL12 proteins disclosed herein, any of the CARs such as anti-CD70 CARs disclosed herein, a disrupted TRAC gene, a disrupted B2M gene, and optionally a disrupted target antigen gene such as CD70 gene as also disclosed herein.

The IL12 expression cassette may be inserted into a genomic site of interest, for example, in the AAVS1 gene, in the B2M gene, or in the target antigen gene such as the CD70 gene. In some examples, the IL12 expression cassette comprise one or more binding sites of the transcriptional regulatory factors disclosed herein such that expression of the IL12 protein is triggered by T cell activation. In some instances, the IL12 expression cassette may be inserted at the site of SEQ ID NO: 48 in the AAVS1 gene. In some instances, the IL12 expression may be inserted at the site of SEQ ID NO: 36 in the B2M gene. In other instances, the IL12 expression cassette may be inserted at the site of SEQ ID NO: 54 in the CD70 gene.

The nucleic acid encoding the anti-CD70 CAR can be inserted in a genomic site of interest, for example, in the disrupted TRAC gene, thereby disrupting expression of the TRAC gene. In some examples, the CAR-coding sequence can be inserted at the site of SEQ ID NO: 22, e.g., replacing a fragment in the TRAC gene that comprise SEQ ID NO: 22.

The population of genetically engineered T cells disclosed herein may be a heterogeneous cell population comprising T cells having one or more of the genetic modifications disclosed herein, for example, expressing the IL12 protein, the anti-CD70 CAR, having a disrupted TRAC gene, having a disrupted B2M gene, or a combination thereof. For example, the cell population may comprises genetically engineered T cells that, collectively (as a whole), exhibit the following genetic modifications: (a) express an exogenous IL12 protein (e.g., activation-dependent), (b) express a CAR, (c) have a disrupted TRAC gene, a disrupted B2M gene, a disrupted target antigen gene, or a combination thereof, while not all of the genetically engineered T cells necessarily exhibit all of the genetic modifications. In some instances, the population of genetically engineered T cells comprise T cells that express an exogenous IL12 in an activation-dependent manner as disclosed herein. Such T cells may express functional TCR. Alternatively, such T cells may express a CAR, the coding sequence of which may be inserted in a TRAC gene locus, thereby disrupting expression of the TRAC gene.

In some examples, at least 30% of a population of the genetically engineered T cells express a detectable level of the IL12 protein. For example, at least 40%, at least 50%, at least 60%, at least 70%, at least 75%, at least 80%, at least 85%, at least 90%, or at least 95% of the genetically engineered T cells express a detectable level of the IL12 protein.

In some examples, at least 30% of a population of the genetically engineered T cells express a detectable level of the anti-CD70 CAR. For example, at least 40%, at least 50%, at least 60%, at least 70%, at least 75%, at least 80%, at least 85%, at least 90%, or at least 95% of the genetically engineered T cells express a detectable level of the anti-CD70 CAR.

In some embodiments, at least 30% of the T cells in the population of genetically engineered T cells may not express a detectable level of β2M surface protein. For example, at least 40%, at least 50%, at least 60%, at least 70% or more of the T cells in the population may not express a detectable level of β2M surface protein.

Alternatively or in addition, at least 50% of the T cells in the population of genetically engineered T cells may not express a detectable level of TCR surface protein. For example, at least 60%, at least 70%, at least 80%, at least 90%, at least 95% or more of the T cells in the population may not express a detectable level of TCR surface protein.

In some embodiments, a substantial percentage of the cells in the population of genetically engineered T cells may comprise more than one gene edit which results in a certain percentage of cells not expressing more than one gene and/or protein. For example, at least 50% of the cells in the population of genetically engineered T cells may not express a detectable level of two surface proteins, e.g., does not express a detectable level of β2M and TRAC proteins. In some examples, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the cells in the population do not express a detectable level of TRAC and B2M surface proteins.

In some embodiments, a substantial percentage of the cells in the population of genetically engineered T cells may express any of the IL12 protein, any of the anti-CD70 CAR, have a disrupted TRAC gene, a disrupted B2M gene, and optionally a disrupted CD70 gene. The expression cassette coding for the anti-CD70 CAR may be inserted in the TRAC gene, thereby disrupting its expression. In some examples, the disrupted TRAC gene comprises a deletion of a fragment comprising the nucleotide sequence of SEQ ID NO: 22. The CAR expression cassette may be inserted at the deletion site, for example, replacing the fragment comprising SEQ ID NO: 22. The expression cassette of the IL12 protein may be inserted in a genomic site of interest, for example, at the B2M gene, at the AAVS1 gene, or at the CD70 gene.

In some examples, the population of anti-CD70 CAR T cells disclosed herein comprise a plurality of genetically engineered T cells each expressing an IL12 protein (e.g., SEQ ID NO: 3 or SEQ ID NO: 4), expressing an anti-CD70 CAR (e.g., SEQ ID NO: 19), and having a disrupted TRAC gene, a disrupted B2M gene, and a disrupted CD70 gene. In some instances, this plurality of genetically engineered T cells may constitute at least 30% (e.g., at least 40%, at least 50, at least 60% or higher) of the population of anti-CD70 CAR T cells.

### II. Preparation of Genetically Engineered Immune Cells

Any suitable gene editing methods known in the art can be used for making the genetically engineered immune cells (e.g., T cells such as human T cells expressing an IL12 protein and optionally a CAR such as an anti-CD70 CAR) disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9). In specific examples, the genetically engineered immune cells such as T cells are produced by the CRISPR technology in combination with homologous recombination using an adeno-associated viral vector (AAV) as a donor template.

(i) CRISPR-Cas9-Mediated Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

(a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 63 provided in Table 3 below.

(b) Guide RNAs (gRNAs)

CRISPR-Cas9-mediated gene editing as described herein includes the use of a guide

RNA or a gRNA. As used herein, a “gRNA” refers to a genome-targeting nucleic acid that can direct the Cas9 to a specific target sequence within a TRAC gene or a β2M gene for gene editing at the specific target sequence. A guide RNA comprises at least a spacer sequence that hybridizes to a target nucleic acid sequence within a target gene for editing, and a CRISPR repeat sequence.

An exemplary gRNA targeting a TRAC gene is provided in SEQ ID NO: 24 or 27. See Table 3 below. See also WO 2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein. When combined with homologous recombination, an exogenous nucleic acid such as a CAR-coding nucleic acid can be inserted into the TRAC gene. In some instances, insertion of the exogenous nucleic acid may disrupt expression of the TRAC gene.

An exemplary gRNA targeting a β2M gene is provided in SEQ ID NO: 40 or 41. See Table 3 below. See also WO 2019/097305A2, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein. When combined with homologous recombination, an exogenous nucleic acid such as an IL12 expression cassette can be inserted into the B2M gene. In some instances, insertion of the exogenous nucleic acid may disrupt expression of the B2M gene.

An exemplary gRNA targeting an AAVS1 gene is provided in SEQ ID NO: 52 or 53 in Table 3 below. In some embodiments, gRNAs targeting the AAVS1 genomic region and Cas9 create breaks in the AAVS1 genomic region resulting Indels in the AAVS1 locus. When combined with homologous recombination, an exogenous nucleic acid such as an IL12 expression cassette can be inserted into the AAVS1 gene.

An exemplary gRNA targeting the CD70 gene is provided in SEQ ID NO: 58 or 59 in Table 3 below. In some embodiments, gRNAs targeting the CD70 genomic region and Cas9 create breaks in the CD70 genomic region resulting Indels in the CD70 locus. When combined with homologous recombination, an exogenous nucleic acid such as an IL12 expression cassette can be inserted into the CD70 gene. In some instances, insertion of the exogenous nucleic acid may disrupt expression of the CD70 gene.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the TRAC target sequence is 5′-AGAGCAACAGTGCTGTGGCC-3′ (SEQ ID NO: 22), then the gRNA spacer sequence is 5′-AGAGCAACAGUGCUGUGGCC-3′ (SEQ ID NO: 25). In another example, if the (32M target sequence is 5′-GCTACTCTCTCTTTCTGGCC-3′ (SEQ ID NO: 36), then the gRNA spacer sequence is 5′-GCUACUCUCUCUUUCUGGCC-3′ (SEQ ID NO: 38). In other examples, when the AAVS1 target site is 5′-GGGGCCACTAGGGACAGGAT-3′ (SEQ ID NO: 48), then the gRNA spacer sequence is 5′-GGGGCCACUAGGGACAGGAU-3′ (SEQ ID NO: 50). In other examples, when the CD70 target site is 5′-GCTTTGGTCCCATTGGTCGC-3′ (SEQ ID NO: 54), then the gRNA spacer sequence is 5′-GCUUUGGUCCCAUUGGUCGC-3′ (SEQ ID NO: 56). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′ NNNNNNNNNNNNNNNNNNNNNRG-3′, the target nucleic acid can be the sequence that corresponds to the Ns, wherein N can be any nucleotide, and the underlined NRG sequence is the S. pyogenes PAM. Examples are provided as SEQ ID NOs: 60-62 (Table 3).

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO2019/215500, the relevant disclosures of each of which are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as a sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of which are herein incorporated by reference for the purposes and subject matter referenced herein.

Table 3 below provides exemplary components for gene editing of TRAC, B2M, AAVS1, and CD70 genes.

TABLE 3
Exemplary Components for Genetic
Modification of Target Genes

| | | SEQ |
|---|---|---|
| | | ID |
| Description | Sequence (5′→3′) | NO |
| TRAC target | AGAGCAACAGTGCTGTGGCC | 22 |
| sequence | | |
| TRAC target | AGAGCAACAGTGCTGTGGCC | 23 |
| sequence with | (TGG) | |
| (PAM) | | |
| TRAC sgRNA | AGAGCAACAGUGCUGUGGCCg | 24 |
| (TA-1) | uuuuagagcuagaaauagcaa | |
| unmodified | guuaaaauaaggcuaguccgu | |
| | uaucaacuugaaaaaguggca | |
| | ccgagucggugcUUUU | |
| TRAC sgRNA | AGAGCAACAGUGCUGUGGCC | 25 |
| spacer | | |
| unmodified | | |
| TRAC sgRNA | A*G*A*GCAACAGUGCUGUGG | 26 |
| spacer | CC | |
| modified | | |
| TRAC sgRNA | A*G*A*GCAACAGUGCUGUGG | 27 |
| (TA-1) | CCguuuuagagcuagaaauag | |
| modified | caaguuaaaauaaggcuaguc | |
| | cguuaucaacuugaaaaagug | |
| | gcaccgagucggugcU*U*U* | |
| | U | |
| TRAC gene-edit | AAGAGCAACAAATCTGACT | 28 |
| TRAC gene-edit | AAGAGCAACAGTGCTGTGCCT | 29 |
| | GGAGCAACAAATCTGACT | |
| | AAGAGCAACAAATCTGACT | |
| TRAC gene-edit | AAGAGCAACAGTGCTGGAGCA | 30 |
| | ACAAATCTGACTAAGAGCAAC | |
| | AAATCTGACT | |
| TRAC gene-edit | AAGAGCAACAGTGCCTGGAGC | 31 |
| | AACAAATCTGACTAAGAGCAA | |
| | CAAATCTGACT | |
| TRAC gene-edit | AAGAGCAACAGTGCTGACTAA | 32 |
| | GAGCAACAAATCTGACT | |
| TRAC gene-edit | AAGAGCAACAGTGCTGTGGGC | 33 |
| | CTGGAGCAACAAATCTGACTA | |
| | AGAGCAACAAATCTGACT | |
| TRAC gene-edit | AAGAGCAACAGTGCTGGCCTG | 34 |
| | GAGCAACAAATCTGACTAAGA | |
| | GCAACAAATCTGACT | |
| TRAC gene-edit | AAGAGCAACAGTGCTGTGTGC | 35 |
| | CTGGAGCAACAAATCTGACTA | |
| | AGAGCAACAAATCTGACT | |
| B2M target | GCTACTCTCTCTTTCTGGCC | 36 |
| sequence | | |
| B2M target | GCTACTCTCTCTTTCTGGCC | 37 |
| sequence with | (TGG) | |
| (PAM) | | |
| B2M sgRNA | GCUACUCUCUCUUUCUGGCC | 38 |
| spacer | | |
| unmodified | | |
| B2M sgRNA | G*C*U*ACUCUCUCUUUCUGG | 39 |
| spacer | CC | |
| modified | | |
| B2M sgRNA | GCUACUCUCUCUUUCUGGCCg | 40 |
| unmodified | uuuuagagcuagaaauagcaa | |
| | guuaaaauaaggcuaguccgu | |
| | uaucaacuugaaaaaguggca | |
| | ccgagucggugcUUUU | |
| B2M sgRNA | G*C*U*ACUCUCUCUUUCUGG | 41 |
| modified | CCguuuuagagcuagaaauag | |
| | caaguuaaaauaaggcuaguc | |
| | cguuaucaacuugaaaaagug | |
| | gcaccgagucggugcU*U*U* | |
| | U | |
| B2M gene-edit | CGTGGCCTTAGCTGTGCTCGC | 42 |
| | GCTACTCTCTCTTTCTGCCTG | |
| | GAGGCTATCCAGCGTGAGTCT | |
| | CTCCTACCCTCCCGCT | |
| B2M gene-edit | CGTGGCCTTAGCTGTGCTCGC | 43 |
| | GCTACTCTCTCTTTCGCCTGG | |
| | AGGCTATCCAGCGTGAGTCTC | |
| | TCCTACCCTCCCGCT | |
| B2M gene-edit | CGTGGCCTTAGCTGTGCTCGC | 44 |
| | GCTACTCTCTCTTTCTGGAGG | |
| | CTATCCAGCGTGAGTCTCTCC | |
| | TACCCTCCCGCT | |
| B2M gene-edit | CGTGGCCTTAGCTGTGCTCGC | 45 |
| | GCTACTCTCTCTTTCTGGATA | |
| | GCCTGGAGGCTATCCAGCGTG | |
| | AGTCTCTCCTACCCTCCCGCT | |
| B2M gene-edit | CGTGGCCTTAGCTGTGCTCGC | 46 |
| | GCTATCCAGCGTGAGTCTCTC | |
| | CTACCCTCCCGCT | |
| B2M gene-edit | CGTGGCCTTAGCTGTGCTCGC | 47 |
| | GCTACTCTCTCTTTCTGTGGC | |
| | CTGGAGGCTATCCAGCGTGAG | |
| | TCTCTCCTACCCTCCCGCT | |
| AAVS1 target | GGGGCCACTAGGGACAGGAT | 48 |
| sequence | | |
| AAVS1 target | GGGGCCACTAGGGACAGGAT | 49 |
| sequence with | (TGG) | |
| (PAM) | | |
| AAVS1 sgRNA | GGGGCCACUAGGGACAGGAU | 50 |
| spacer | | |
| unmodified | | |
| AAVS1 sgRNA | G*G*G*GCCACUAGGGACAG | 51 |
| spacer | GAU | |
| modified | | |
| AAVS1 sgRNA | GGGGCCACTAGGGACAGGAT | 52 |
| unmodified | guuuuagagcuagaaauagc | |
| | aaguuaaaauaaggcuaguc | |
| | cguuaucaacuugaaaaagu | |
| | ggcaccgagucggugcUUUU | |
| AAVS1 sgRNA | G*G*G*GCCACTAGGGACAG | 53 |
| modified | GATguuuuagagcuagaaau | |
| | agcaaguuaaaauaaggcua | |
| | guccguuaucaacuugaaaa | |
| | aguggcaccgagucggugcU | |
| | UUU | |
| CD70 sgRNA | GCTTTGGTCCCATTGGTCGC | 54 |
| target | | |
| sequence | | |
| CD70 sgRNA | GCTTTGGTCCCATTGGTCGC | 55 |
| target | (TGG) | |
| sequence | | |
| with | | |
| (PAM) | | |
| CD70 sgRNA | GCUUUGGUCCCAUUGGUCGC | 56 |
| spacer | | |
| unmodified | | |
| CD70 sgRNA | G*C*U*UUGGUCCCAUUGGU | 57 |
| modified | CGC | |
| CD70 sgRNA | GCUUUGGUCCCAUUGGUCGC | 58 |
| unmodified | guuuuagagcuagaaauagc | |
| | aaguuaaaauaaggcuaguc | |
| | cguuaucaacuugaaaaagu | |
| | ggcaccgagucggugcUUUU | |
| CD70 sgRNA | G*C*U*UUGGUCCCAUUGGU | 59 |
| modified | CGCguuuuagagcuagaaau | |
| | agcaaguuaaaauaaggcua | |
| | guccguuaucaacuugaaaa | |
| | aguggcaccgagucggugcU | |
| | UUU | |
| sgRNA | nnnnnnnnnnnnnnnnnnnn | 60 |
| | guuuuagagcuagaaauagc | |
| | aaguuaaaauaaggcuaguc | |
| | cguuaucaacuugaaaaagu | |
| | ggcaccgagucggugcuuuu | |
| sgRNA | nnnnnnnnnnnnnnnnnnnn | 61 |
| | guuuuagagcuagaaauagc | |
| | aaguuaaaauaaggcuaguc | |
| | cguuaucaacuugaaaaagu | |
| | ggcaccgagucggugc | |
| sgRNA | n₍₁₇₋₃₀₎guuuuagagcua | 62 |
| | gaaauagcaaguuaaaauaa | |
| | ggcuaguccguuaucaacuu | |
| | gaaaaaguggcaccgagucg | |
| | gugcu₍₁₋₈₎ | |
| spCas9 | MDKKYSIGLDIGTNSVGWAV | 63 |
| | ITDEYKVPSKKFKVLGNTDR | |
| | HSIKKNLIGALLFDSGETAE | |
| | ATRLKRTARRRYTRRKNRIC | |
| | YLQEIFSNEMAKVDDSFFHR | |
| | LEESFLVEEDKKHERHPIFG | |
| | NIVDEVAYHEKYPTIYHLRK | |
| | KLVDSTDKADLRLIYLALAH | |
| | MIKFRGHFLIEGDLNPDNSD | |
| | VDKLFIQLVQTYNQLFEENP | |
| | INASGVDAKAILSARLSKSR | |
| | RLENLIAQLPGEKKNGLFGN | |
| | LIALSLGLTPNFKSNFDLAE | |
| | DAKLQLSKDTYDDDLDNLLA | |
| | QIGDQYADLFLAAKNLSDAI | |
| | LLSDILRVNTEITKAPLSAS | |
| | MIKRYDEHHQDLTLLKALVR | |
| | QQLPEKYKEIFFDQSKNGYA | |
| | GYIDGGASQEEFYKFIKPIL | |
| | EKMDGTEELLVKLNREDLLR | |
| | KQRTFDNGSIPHQIHLGELH | |
| | AILRRQEDFYPFLKDNREKI | |
| | EKILTFRIPYYVGPLARGNS | |
| | RFAWMTRKSEETITPWNFEE | |
| | VVDKGASAQSFIERMTNFDK | |
| | NLPNEKVLPKHSLLYEYFTV | |
| | YNELTKVKYVTEGMRKPAFL | |
| | SGEQKKAIVDLLFKTNRKVT | |
| | VKQLKEDYFKKIECFDSVEI | |
| | SGVEDRFNASLGTYHDLLKI | |
| | IKDKDFLDNEENEDILEDIV | |
| | LTLTLFEDREMIEERLKTYA | |
| | HLFDDKVMKQLKRRRYTGWG | |
| | RLSRKLINGIRDKQSGKTIL | |
| | DFLKSDGFANRNFMQLIHDD | |
| | SLTFKEDIQKAQVSGQGDSL | |
| | HEHIANLAGSPAIKKGILQT | |
| | VKVVDELVKVMGRHKPENIV | |
| | IEMARENQTTQKGQKNSRER | |
| | MKRIEEGIKELGSQILKEHP | |
| | VENTQLQNEKKLYLYYLQNGR | |
| | DMYVDQELDINRLSDYDVDHI | |
| | VPQSFLKDDSIDNKVLTRSDK | |
| | NRGKSDNVPSEEWKKMKNYWR | |
| | QLLNAKLITQRKFDNLTKAER | |
| | GGLSELDKAGFIKRQLVETRQ | |
| | ITKHVAQILDSRMNTKYDEND | |
| | KLIREVKVITLKSKLVSDFRK | |
| | DFQFYKVREINNYHHAHDAYL | |
| | NAWGTALIKKYPKLESEFVYG | |
| | DYKVYDVRKMIAKSEQEIGKA | |
| | TAKYFFYSNIMNFFKTEITLA | |
| | NGEIRKRPLIETNGETGEIVW | |
| | DKGRDFATVRKVLSMPQVNIV | |
| | KKTEVQTGGFSKESILPKRNS | |
| | DKLIARKKDWDPKKYGGFDSP | |
| | TVAYSVLWAKVEKGKSKKLKS | |
| | VKELLGITIMERSSFEKNPID | |
| | FLEAKGYKEVKKDLIIKLPKY | |
| | SLFELENGRKRMLASAGELQK | |
| | GNELALPSKYVNFLYLASHYE | |
| | KLKGSPEDNEQKQLFVEQHKH | |
| | YLDEIIEQISEFSKRVILADA | |
| | NLDKVLSAYNKHRDKPIREQA | |
| | ENIIHLFTLTNLGAPAAFKYF | |
| | DTTIDRKRYTSTKEVLDATLI | |
| | HQSITGLYETRIDLSQLGGD | |

*indicates a nucleotide with a 2′-O-methyl phosphorothioate modification.
“n” refers to the spacer sequence at the 5′ end.

(ii) AAV Vectors for Delivery of CAR Constructs and IL12 Expression Cassettes to T Cells

A nucleic acid encoding any of the IL12 constructs and/or any of the CAR constructs such as anti-CD70 CAR constructs as disclosed herein can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

In some embodiments, a nucleic acid encoding any of the CAR construct such as an anti-CD70 CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions, and inserting a CAR coding segment into the TRAC gene.

In some embodiments, an IL12 expression cassettes such as those disclosed herein (e.g., via a donor template, which can be carried by a viral vector such as an adeno-associated viral (AAV) vector) can be designed such that it can insert into a location within a target gene of interest, e.g., the AAVS1 gene, the B2M gene, or the target antigen gene such as the CD70 gene, to disrupt the target gene in the genetically engineered T cells and express the IL12 polypeptide. Disruption of the target gene may lead to loss of function of the endogenous target gene. For example, a disruption in the B2M gene can be created with an endonuclease such as those described herein and one or more gRNAs targeting one or more B2M genomic regions, thereby disrupting expression of MHC Class I molecules. Any of the gRNAs specific to the AAVS1 gene, the B2M gene, and/or the CD70 gene and the target regions can be used for this purpose, e.g., those disclosed herein.

In some examples, a genomic deletion in the target gene (AAVS1, B2M or target antigen gene such as CD70 gene) and replacement by an IL12 coding segment can be created by homology directed repair or HDR (e.g., using a donor template, which may be part of a viral vector such as an adeno-associated viral (AAV) vector). In some embodiments, a disruption in the target gene can be created with an endonuclease as those disclosed herein and one or more gRNAs targeting one or more target genomic regions, and inserting an IL12 expression cassette into the target gene. See Examples below.

A donor template as disclosed herein can contain a coding sequence for a CAR. In some examples, the CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLY)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

### III. Pharmaceutical Compositions and Therapeutic Applications

In another aspect, provided herein are therapeutic applications of any of the genetically engineered immune cells such as T cells disclosed herein that express an IL12 protein and a CAR such as a CAR targeting a tumor antigen (e.g., an anti-CD70 CA). Such therapeutic applications include eliminating disease cells expressing the antigen targeted by the CAR construct, for example, CD70⁺ cancer cells.

Any of the genetically engineered immune cells such as T cells as disclosed herein (e.g., those expressing exogenous IL12 and CAR as also disclosed herein and having one or more additional genetic edits such as a disrupted TRAC gene, a disrupted B2M gene, and/or a disrupted target antigen gene) may be formulated in a pharmaceutical composition, which may further comprise one or more pharmaceutically acceptable excipients. Such pharmaceutical compositions are also within the scope of the present disclosure. The pharmaceutical compositions can be used in therapeutic applications, for example, cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid, or an organic acid. In some embodiments, the salt formed with the free carboxyl groups is derived from an inorganic base, or an organic base. In some embodiments, the pharmaceutical composition disclosed herein comprises a population of the genetically engineered CAR-T cells disclosed herein such as the anti-CD70 CAR-T cells may be suspended in a cryopreservation solution (e.g., CryoStor C55).

Any of the genetically engineered immune cells such as T cells disclosed herein may be used for treating cancer, for example, a solid cancer or a hematopoietic cancer such as a T cell or B cell malignancy. Thus, provided herein are methods for treating cancer comprising administering to a subject in need of the treatment an effective amount of the genetically engineered immune cells.

The step of administering CAR T cell therapy may include the placement (e.g., transplantation) of cells, e.g., engineered human CAR T cells, into a subject, by a method or route that results in at least partial localization of the introduced cells at a desired site, such as tumor, such that a desired effect(s) is produced. Engineered T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of engineered T cells is administered via a systemic route of administration, such as an intraperitoneal or intravenous route.

A subject may be any subject for whom treatment or therapy is desired. In some embodiments, the subject is a mammal. In some embodiments, the subject is a human.

In some embodiments, any of the genetically engineered T cells expression an anti-CD70 CAR and an exogenous IL12 protein (encoded by any of the IL12 expression cassettes disclosed herein), as well as having one or more of the disrupted endogenous genes such as TRAC and B2M as disclosed herein, can be used for treating a cancer that carries CD70⁺ cancer cells.

A human patient to be treated by the methods described herein can be a human patient having, suspected of having, or a risk for having a solid tumor. Non-limiting examples of solid tumors include pancreatic cancer, gastric cancer, ovarian cancer, cervical cancer, breast cancer, renal cancer, thyroid cancer, nasopharyngeal cancer, non-small cell lung (NSCLC), glioblastoma, and/or melanoma.

In some examples, the solid tumor is renal cell carcinoma (RCC). A subject suspected of having RCC might show one or more symptoms of RCC, e.g., unexplained weight loss, anemia, abdominal pain, blood in the urine, or lumps in the abdomen. A subject at risk for RCC can be a subject having one or more of the risk factors for RCC, e.g., smoking, obesity, high blood pressure, family history of RCC, or genetic conditions such as von Hippel-Lindau disease. A human patient who needs the anti-CD70 CAR T cell treatment may be identified by routine medical examination, e.g., laboratory tests, biopsy, magnetic resonance imaging (MRI) scans, or ultrasound exams

In some examples, the solid tumor is lung cancer such as non-small cell lung cancer (NSCLC). A subject suspected of having lung cancer such as NSCLC might show one or more symptoms of the lung cancer, e.g., unexplained weight loss, pain in the back and/or chest, cough (chronic or with blood), shortness of breath or wheezing, phlegm, and/or pneumonia. A subject at risk for lung cancer such as NSCLC can be a subject having one or more of the risk factors, e.g., smoking, exposure to asbestos or radon, family history of NSCLC, or genetic conditions such as mutations in the EFGR gene. A human patient who needs the anti-CD70 CAR T cell treatment may be identified by routine medical examination, e.g., laboratory tests, biopsy, magnetic resonance imaging (MRI) scans, or ultrasound exams

Examples of renal cell carcinomas (RCCs) that may be treated using methods described herein include, but are not limited to, clear cell renal carcinomas (ccRCC), papillary renal cell carcinomas (pRCC), and chromophobe renal cell carcinomas (crRCC). These three subtypes account for more than 90% of all RCCs.

In some embodiments, the human patient has unresectable or metastatic RCC. In some embodiments, the human patient has predominantly clear cell RCC (ccRCC). In some embodiments, the human patient has unresectable or metastatic RCC with predominantly clear cell differentiation.

In some examples, the solid tumor is pancreatic cancer such as pancreatic adenocarcinoma, pancreatic systic tumor, pancreatic acinar cell cancer, pancreatic sarcoma, or pancreatic ampullary cancer. A subject suspected of having pancreatic cancer may exhibit one or more symptoms associated with pancreatic cancer, for example, upper abdomen pain, diarrhea, flushing of the skin and face, hypoglycemia or hyperglycemia, digestive problems, gallbladder proteins, change in weight, or a combination thereof. A subject at risk for pancreatic cancer can be a subject having one or more of the risk factors, e.g., age, increased body mass index, smoking, diabetes, and/or chronic inflammation.

In other embodiments, a human patient to be treated by the methods described herein can be a human patient having, suspected of having, or a risk for having hematopoietic malignancies, such as a T cell or B cell malignancy. A subject suspected of having a T cell or B cell malignancy might show one or more symptoms of T cell or B cell malignancy, e.g., unexplained weight loss, fatigue, night sweats, shortness of breath, or swollen glands. A subject at risk for T cell or B cell malignancy can be a subject having one or more of the risk factors for T cell or B cell malignancy, e.g., a weakened immune system, age, male, or infection (e.g., Epstein-Barr virus infection). A human patient who needs the anti-CD70 CAR T cell treatment may be identified by routine medical examination, e.g., physical examination, laboratory tests, biopsy (e.g., bone marrow biopsy and/or lymph node biopsy), magnetic resonance imaging (MRI) scans, or ultrasound exams

Examples of T cell and B cell malignancies that may be treated using the methods described herein include, but are not limited to, peripheral T cell lymphoma (PTCL), anaplastic large cell lymphoma (ALCL), Sezary syndrome (SS), non-smoldering acute adult T cell leukemia or lymphoma (ATLL), angioimmunoblastic T cell lymphoma (AITL), and diffuse large B cell lymphoma (DLBCL).

In some embodiments, an engineered human CAR T cell population being administered according to the methods described herein does not induce toxicity in the subject, e.g., the engineered human CAR T cells do not induce toxicity in non-cancer cells. In some embodiments, an engineered human CAR T cell population being administered does not trigger complement mediated lysis or does not stimulate antibody-dependent cell mediated cytotoxicity (ADCC). In some embodiments, an engineered human CAR T cell population being administered does not trigger apoptosis. In some embodiments, an engineered human CAR T cell population being administered does not trigger ADCP.

An effective amount refers to the amount of a population of engineered human CAR T cells needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments, a subject is administered a population of cells comprising engineered T cells (e.g., engineered human CAR T cells) at a dose of about 1×10⁷ to about 1×10⁹ engineered T cells expressing a detectable level of CAR described herein.

In some embodiments, an engineered human CAR T cell population being administered according to the methods described herein comprises allogeneic T cells obtained from one or more donors, for example, healthy human donors. Allogeneic refers to a cell, cell population, or biological samples comprising cells, obtained from one or more different donors of the same species, where the genes at one or more loci are not identical to the recipient. For example, an engineered CAR T cell population, being administered to a subject can be derived from T cells from one or more unrelated donors, or from one or more non-identical siblings. In some embodiments, syngeneic cell populations may be used, such as those obtained from genetically identical donors, (e.g., identical twins). In some examples described herein, the cells are expanded in culture prior to administration to a subject in need thereof.

Modes of administration for engineered human CAR T cells include injection and infusion. Injection includes, without limitation, intravenous, intrathecal, intraperitoneal, intraspinal, intracerebro spinal, and intrasternal infusion. In some embodiments, the route is intravenous.

In some embodiments, engineered human CAR T cells are administered systemically, which refers to the administration other than directly into a target site, tissue, or organ, such that it enters, instead, the subject's circulatory system and, thus, is subject to metabolism and other like processes.

The clinical outcome of a treatment comprising a composition for the treatment of a medical condition can be determined by the skilled clinician. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Clinical outcome can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

Prior to administration of any of the genetically engineered T cells disclosed herein, the subject may be treated by a lymphodepletion regimen (e.g., a conventional lymphodepletion regimen) to condition the subject for the T cell therapy. Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

### III. Kits for Use in Cancer Therapy

The present disclosure also provides kits for use of a population of the genetically engineered T cells disclosed herein, such as the anti-CD70 CAR T cells, in methods for treating solid tumors. Such kits may include one or more containers comprising a pharmaceutical composition that comprises any population of the genetically engineered T cells (e.g., those described herein), and a pharmaceutically acceptable carrier, and optionally one or more pharmaceutical compositions that comprises one or more lymphodepleting agents.

In some embodiments, the kit can comprise instructions for use in any of the methods described herein. The included instructions can comprise a description of administration of the genetically engineered T cells and optionally the lymphodepletion agents to a subject to achieve the intended therapeutic effects. The kit may further comprise a description of selecting a human patient suitable for treatment based on identifying whether the human patient is in need of the treatment, for example, identifying a human patient carrying CD70+ cancer cells. In some embodiments, the instructions comprise a description of administering the pharmaceutical compositions contained in the kit to a human patient who is in need of the treatment.

The instructions relating to the use of the population of genetically engineered T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a renal cell carcinoma in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the genetically engineered immune cells such as the anti-CD70 CAR-T cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning:A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R.I. Freshney, ed. (1986»; Immobilized Cells and Enzymes (1RL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1: Constructs Designed to Express a Transgene Upon T Cell Activation

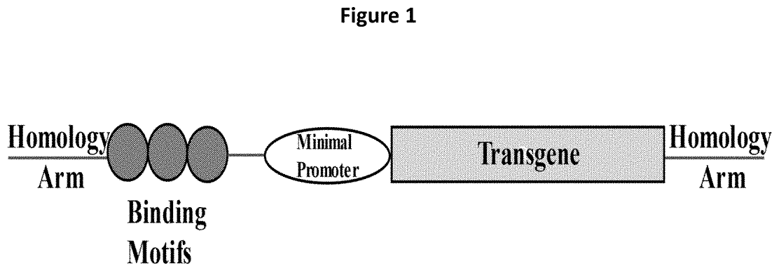

To generate T cells that express transgenes only upon activation, activation-dependent constructs were generated. Specifically, the DNA double-stranded break at the AAVS1 locus was repaired by homology-directed repair with activation-dependent recombinant adeno-associated adenoviral vectors, serotype 6 (AAV6) comprising similar nucleotide sequences with differences in the binding motifs and the minimal promoter regions and also containing right homology arms (RHAs) and left homology arms (LHAs) for targeted insertion of the donor sequence into a specific loci (e.g.: the AAVS1 locus). The general structure of an activation-dependent construct is shown in FIG. 1.

Twelve constructs (CTX936-CTX947) were generated using this general structure using the transgene dsGFP and AAVS1 LHAs and RHAs. Five different binding motifs including NF-κB, NFAT, AP1, STAT5 and SMAD responsive elements and two different minimal promoters—min-IL2 or Late ADE—were tested for the activation-dependent expression of a transgene (e.g.: destabilized GFP [dsGFP]). Sequences of the components used for generating the CTX936-CTX947 constructs are provided in Table 4.

TABLE 4
Sequences of Construct Components

| | SEQ | |
|---|---|---|
| | ID | |
| Name | NO: | SEQUENCE |
| dsGFP | 64 | MVSKGEELFTGVVPILVELDGDVNGHKFSVSGEGEGD |
| | | ATYGKLTLKFICTTGKLPVPWPTLVTTLTYGVQCFSR |
| | | YPDHMKQHDFFKSAMPEGYVQERTIFFKDDGNYKTRA |
| | | EVKFEGDTLVNRIELKGIDFKEDGNILGHKLEYNYNS |
| | | HNVYIMADKQKNGIKVNFKIRHNIEDGSVQLADHYQQ |
| | | NTPIGDGPVLLPDNHYLSTQSALSKDPNEKRDHMVLL |
| | | EFVTAAGITLGMDELYKSGLRSLMKQIQSHGFPPEVE |
| | | EQDDGTLPMSCAQESGMDRHPAACASARINV |
| NF-κB | 65 | TGGGGATTCCCCA |
| binding | | |
| motif | | |
| NFAT | 66 | GGAGGAAAAACTGTTTCATACAGAAGGCGT |
| binding | | |
| motif | | |
| AP1 | 67 | TGACTCA |
| binding | | |
| motif | | |
| STAT5 | 68 | TTCTGAGAA |
| binding | | |
| motif | | |
| SMAD | 69 | GTCTAGAC |
| binding | | |
| motif | | |
| Min IL2 | 70 | CATTTTGACACCCCCATAATATTTTTCCAGAATTAA |
| promoter | | CAGTATAAATTGCATCTCTTGTTCAAGAGTTCCCTA |
| | | TCACTCTCTTTAATCACTACTCACAGTAACCTCAAC |
| | | TCCTGC |
| Late ADE | 71 | AGACGCTAGCGGGGGGCTATAAAAGGGGGTGGGGG |
| promoter | | CGTTCGTCCTCACTCT |
| AAVS1 | 72 | ACTGTGGGGTGGAGGGGACAGATAAAAGTACCCAGA |
| RHA | | ACCAGAGCCACATTAACCGGCCCTGGGAATATAAGG |
| | | TGGTCCCAGCTCGGGGACACAGGATCCCTGGAGGCA |
| | | GCAAACATGCTGTCCTGAAGTGGACATAGGGGCCCG |
| | | GGTTGGAGGAAGAAGACTAGCTGAGCTCTCGGACCC |
| | | CTGGAAGATGCCATGACAGGGGGCTGGAAGAGCTAG |
| | | CACAGACTAGAGAGGTAAGGGGGGTAGGGGAGCTG |
| | | CCCAAATGAAAGGAGTGAGAGGTGACCCGAATCCAC |
| | | AGGAGAACGGGGTGTCCAGGCAAAGAAAGCAAGAG |
| | | GATGGAGAGGTGGCTAAAGCCAGGGAGACGGGGTA |
| | | CTTTGGGGTTGTCCAGAAAAACGGTGATGATGCAGG |
| | | CCTACAAGAAGGGGAGGCGGGACGCAAGGGAGACA |
| | | TCCGTCGGAGAAGGCCATCCTAAGAAACGAGAGATG |
| | | GCACAGGCCCCAGAAGGAGAAGGAAAAGGGAACCC |
| | | AGCGAGTGAAGACGGCATGGGGTTGGGTGAGGGAG |
| | | GAGAGATGCCCGGAGAGGACCCAGACACGGGGAGG |
| | | ATCCGCTCAGAGGACATCACGTGGTGCAGCGCCGAG |
| | | AAGGAAGTGCTCCGGAAAGAGCATCCTTGGGCAGCA |
| | | ACACAGCAGAGAGCAAGGGGAAGAGGGAGTGGAGG |
| | | AAGACGGAACCTGAAGGAGGCGGC |
| AAVS1 | 73 | GAAGCCCAGAGCAGGGCCTTAGGGAAGCGGGACCCT |
| LHA | | GCTCTGGGCGGAGGAATATGTCCCAGATAGCACTGG |
| | | GGACTCTTTAAGGAAAGAAGGATGGAGAAAGAGAA |
| | | AGGGAGTAGAGGCGGCCACGACCTGGTGAACACCTA |
| | | GGACGCACCATTCTCACAAAGGGAGTTTTCCACACG |
| | | GACACCCCCCTCCTCACCACAGCCCTGCCAGGACGG |
| | | GGCTGGCTACTGGCCTTATCTCACAGGTAAAACTGA |
| | | CGCACGGAGGAACAATATAAATTGGGGACTAGAAAG |
| | | GTGAAGAGCCAAAGTTAGAACTCAGGACCAACTTAT |
| | | TCTGATTTTGTTTTTCCAAACTGCTTCTCCTCTTGG |
| | | GAAGTGTAAGGAAGCTGCAGCACCAGGATCAGTGAA |
| | | ACGCACCAGACGGCCGCGTCAGAGCAGCTCAGGTTC |
| | | TGGGAGAGGGTAGCGCAGGGTGGCCACTGAGAACCGG |
| | | GCAGGTCACGCATCCCCCCCTTCCCTCCCACCCCCTG |
| | | CCAAGCTCTCCCTCCCAGGATCCTCTCTGGCTCCATC |
| | | GTAAGCAAACCTTAGAGGTTCTGGCAAGGAGAGAGA |
| | | TGGCTCCAGGAAATGGGGGTGTGTCACCAGATAAGG |
| | | AATCTGCCTAACAGGAGGTGGGGGTTAGACCCAATA |
| | | TCAGGAGACTAGGAAGGAGGAGGCCTAAGGATGGG |
| | | GCTTTTCTGTCACCA |

Table 5 provides the structure and nucleic acid sequences of the CTX936-CTX947 constructs.

TABLE 5
CTX936-CTX947 Construct Structures and Sequences

| | Structure | |
|---|---|---|
| Construct | (LHA - Binding Domain (optional*) - Promoter - | SEQ |
| ID | Transgene- RHA) | ID NO: |
| CTX936* | AAVS1 LHA - min IL2 - dsGFP - AAVS1 RHA | 74 |
| CTX937 | AAVS1 LHA-3x NFKb-min IL2-dsGFP- AAVS1 RHA | 75 |
| CTX938 | AAVS1 LHA-3x NFAT-min IL2-dsGFP- AAVS1 RHA | 76 |
| CTX939 | AAVS1 LHA-3x AP1-min IL2-dsGFP- AAVS1 RHA | 77 |
| CTX940 | AAVS1 LHA-5x STAT5-min IL2-dsGFP- AAVS1 RHA | 78 |
| CTX941 | AAVS1 LHA-4x SMAD-min IL2-dsGFP- AAVS1 RHA | 79 |
| CTX942* | AAVS1 LHA-late ADE-dsGFP- AAVS1 RHA | 80 |
| CTX943 | AAVS1 LHA-3x NFKb-late ADE-dsGFP- AAVS1 RHA | 81 |
| CTX944 | AAVS1 LHA -3x NFAT-late ADE-dsGFP- AAVS1 RHA | 82 |
| CTX945 | AAVS1 LHA -3X AP1-late ADE-dsGFP- AAVS1 RHA | 83 |
| CTX946 | AAVS1 LHA -5x STAT6-late ADE-dsGFP- AAVS1 RHA | 84 |
| CTX947 | AAVS1 LHA -4x SMAD-late ADE-dsGFP- AAVS1 RHA | 85 |

*construct does not contain a binding domain sequence

The AAV6s were delivered with Cas9:sgRNA ribonucleoproteins (RNPs) (1 μM Cas9, 5 μM gRNA) by electroporation into activated human T cells using methods similar to that described in Hendel et al., Nat Biotechnol. 2015; 33(9):985-989, the disclosure of which is incorporated herein in its entirety. Briefly, T cells were isolated from human subjects. Next, isolated human T cells were electroporated using a Lonza Nucleofector device in a nucleofection mix. The nucleofection mix contained Nucleofector™ Solution, 5×10⁶ cells, 1 μM spCas9, and 5 μM gRNA. The RNP complex comprised Cas9 and one sgRNA targeting SEQ ID NO: 48 in AAVS1. AAVS1 sgRNAs included SEQ ID NOs: 52-53. (See Table 3 for sequences).

About one week post-nucleofection, edited T cells either remained untreated (resting) or were treated with phorbol myristate acetate (PMA)/ionomycin for four hours (stimulated). Then, cells were processed for flow cytometry to assess dsGFP expression levels at the cell surface of the edited cell populations.

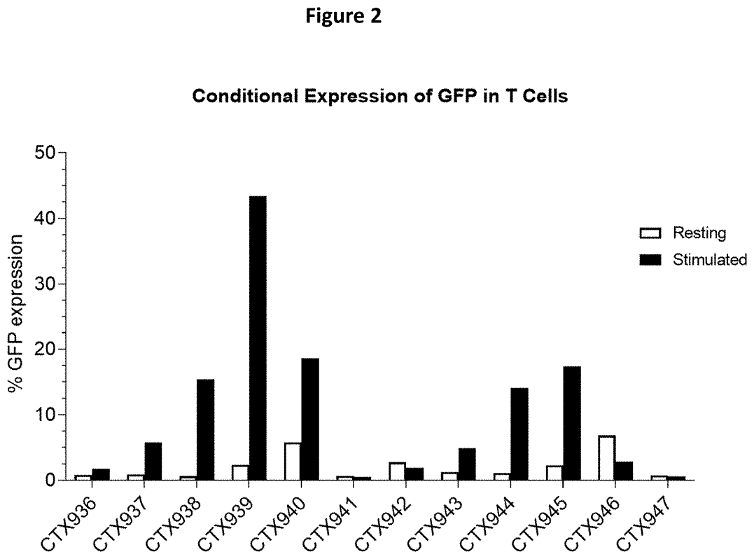

As shown in FIG. 2, edited T cell expression of dsGFP upon stimulation with PMA/ionomycin varied by construct. Specifically, T cells containing the CTX939 construct, which contained the AP1 binding motif and minimal IL-2 promoter, gave the highest level of dsGFP expression upon stimulation.

A similar study was conducted with CAR T cells. Expression of dsGFP was induced with stimulation with PMA/ionomycin. Similar results were observed in CAR-T cells relative to T cells.

### Example 2: Generation of CD70 CAR T Cells that Conditionally Expressed a Transgene Upon Engagement with Target Antigen

Allogeneic human T cells that lacked expression of the TRAC gene, β2M gene, CD70 gene and AAVS1 gene, and expressed a chimeric antigen receptor (CAR) targeting CD70 in addition to a conditional transgene were produced. Briefly, human T cells were first isolated and then subjected to electroporation in the presence of two recombinant adeno-associated adenoviral vectors (AAVs), serotype 6 (AAV6) (MOI 50,000), and Cas9:sgRNA RNPs (1 μM Cas9, 5 μM gRNA), using the method described in Example 1 above. Of the two AAVs electroporated into the cells, one recombinant AAV included the nucleotide sequence encoding an anti-CD70 CAR (the donor template in SEQ ID NO: 21 and the anti-CD70 CAR amino acid sequence of SEQ ID NO: 19). The second recombinant AAV was one of the activation-dependent constructs described in Table 5 (CTX936-CTX947). In addition, the following sgRNAs were also used: TRAC (SEQ ID NO: 27), β2M (SEQ ID NO: 41), CD70 (SEQ ID NO: 59) and AAVS1 (SEQ ID NO: 53). Table 3 above provides the nucleic acid sequences of the sgRNAs that were used in this study in addition to target sequences and spacers for the sgRNAs.

About one week post electroporation, the T cells either remained untreated (resting) or were stimulated by co-culturing the cells with A498 cells that express CD70 target antigen for the anti-CD70 CAR T. Specifically, A498 cells expressing the CD70 target antigen were co-cultured overnight with edited T cells containing one of the activation-dependent constructs described in Example 1 (CTX936-CTX947). Then, the cells were processed for flow cytometry to assess dsGFP expression levels at the cell surface of the edited cell population.

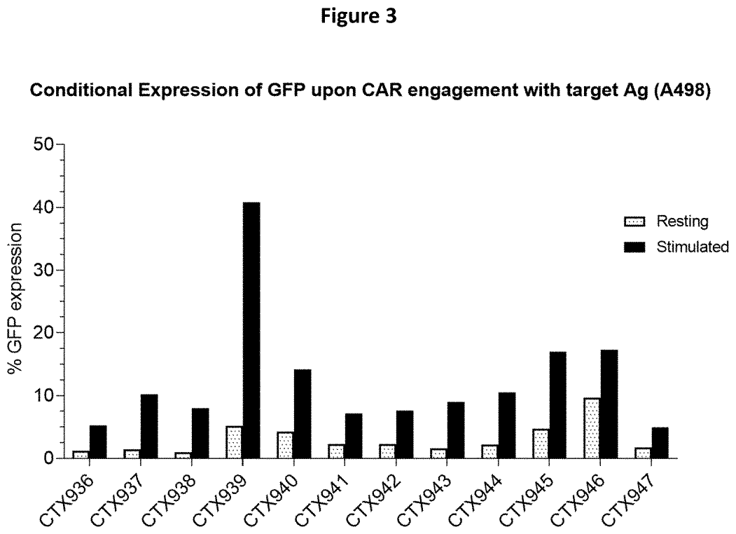

Edited CAR T cell expression of dsGFP upon stimulation with A498 cells varied by construct. As shown in FIG. 3, edited CAR T cells electroporated with the constructs demonstrated expression of the transgene upon T cells activation following antigen exposure. Edited CAR-T cells carrying the CTX939 construct showed the highest expression level of the transgene.

### Example 3: Generation of Genetically Modified T Cells That Conditionally Expressed an IL12 Transgene Upon T cell Activation

The design of inducible (activation-dependent) IL12 constructs was based on the CTX939 construct described in the previous examples. Specifically, CTX939 was modified to replace the dsGFP reporter transgene with one of two splice variants of human IL12 (SEQ ID NO: 3 and SEQ ID NO: 4).

Additional modifications were made to the homology arms to selectively insert the transgene into difference loci. Specifically, homology arms were designed to three loci: AAVS1 (CTX1560-CTX1563), B2M (CTX1564-CTX1567) or CD70 (CTX1568-CTX1571). The nucleic acid sequences of the homology arms designed to the AAVS1 locus are provided in Table 7 (SEQ ID NOs: 72-73) and the nucleic acid sequences of the homology arms designed to the B2M and CD70 loci are provided in Table 7 below.

Table 6 discloses the structures of the CTX1560-CTX1571 constructs and Table 7 discloses the nucleic acid sequences of the CTX1560-CTX1571 constructs.

TABLE 6
Structures and Sequences of the CTX1560-CTX1571

| | Structure | |
|---|---|---|
| Construct | (LHA - Binding Domain (optional*) - Promoter - | SEQ |
| ID | Transgene- RHA) | ID NO: |
| CTX1560* | AAVS1 LHA - Min IL2 promoter - hIL12 (902) - AAVS1 RHA | 90 |
| CTX1561 | AAVS1 LHA -3X AP - Min IL2 promoter- hIL12 (902)- AAVS1 | 91 |
| | RHA | |
| CTX1562* | AAVS1 LHA - Min IL2 promoter - hIL12 (901) - AAVS1 RHA | 92 |
| CTX1563 | AAVS1 LHA -3X AP - Min IL2 promoter -hIL12 (901)- AAVS1 | 93 |
| | RHA | |
| CTX1564* | B2M LHA - Min IL2 promoter - hIL12 (902) - B2M RHA | 94 |
| CTX1565 | B2M LHA -3XAP - Min IL2 promoter - hIL12 (902) - B2M RHA | 95 |
| CTX1566* | B2M LHA - Min IL2 promoter - hIL12 (901) - B2M RHA | 96 |
| CTX1567 | B2M LHA -3XAP - Min IL2 promoter - hIL12 (901) - B2M RHA | 97 |
| CTX1568* | CD70 LHA - Min IL2 promoter - hIL12 (902) - CD70 RHA | 98 |
| CTX1569 | CD70 LHA -3XAP - Min IL2 promoter - hIL12 (902) - CD70 RHA | 99 |
| CTX1570* | CD70 LHA - Min IL2 promoter - hIL12 (901) - CD70 RHA | 100 |
| CTX1571 | CD70 LHA -3XAP - Min IL2 promoter - hIL12 (901) - CD70 RHA | 101 |

*construct does not contain a binding domain sequence

The CTX1560-CTX1571 constructs were next tested in T cells for the expression of IL12 upon chemical activation. Briefly, the AAV6 vectors containing one of the constructs selected from CTX1560-CTX1571 were delivered with Cas9:sgRNA RNPs (1 μM Cas9, 5 μM gRNA) to a isolated human T cells by electroporation using the method described in Example 1. The nucleofection mix contained the Nucleofector™ Solution, 5×10⁶ cells, 1 μM Cas9, and 5 μM gRNA. The following sgRNAs were used: β2M (SEQ ID NO: 41), CD70 (SEQ ID NO: 59) and AAVS1 (SEQ ID NO: 53).

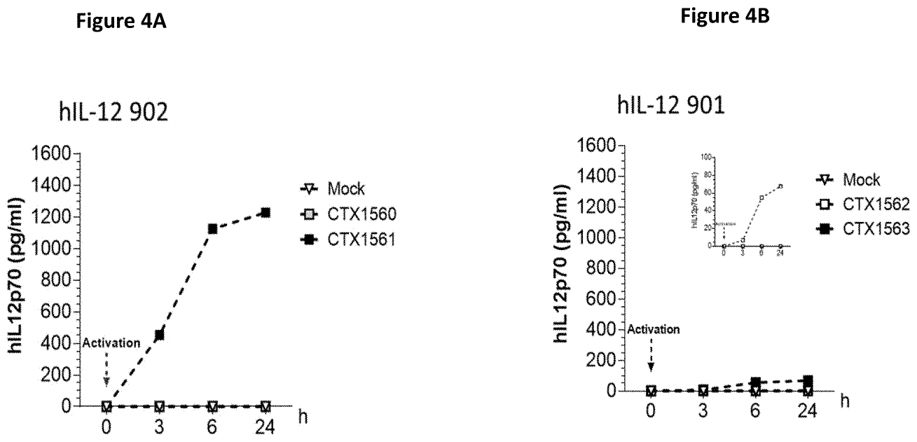

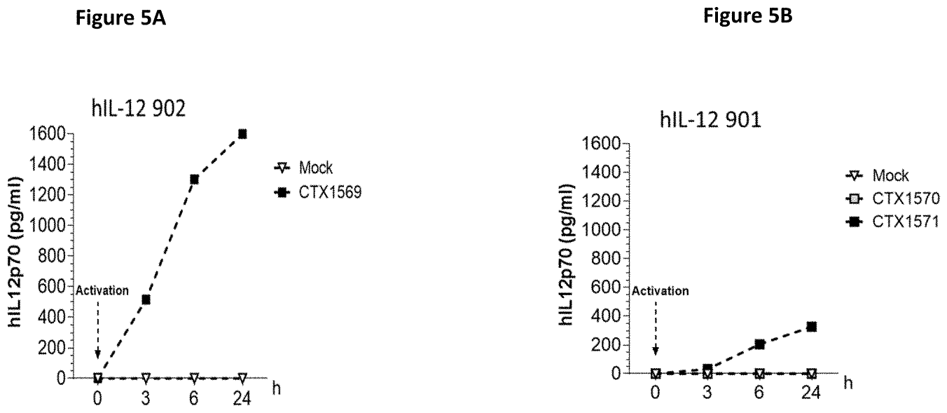

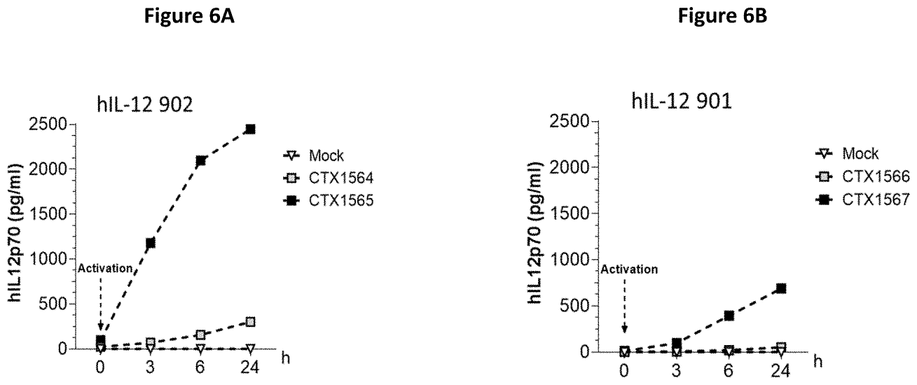

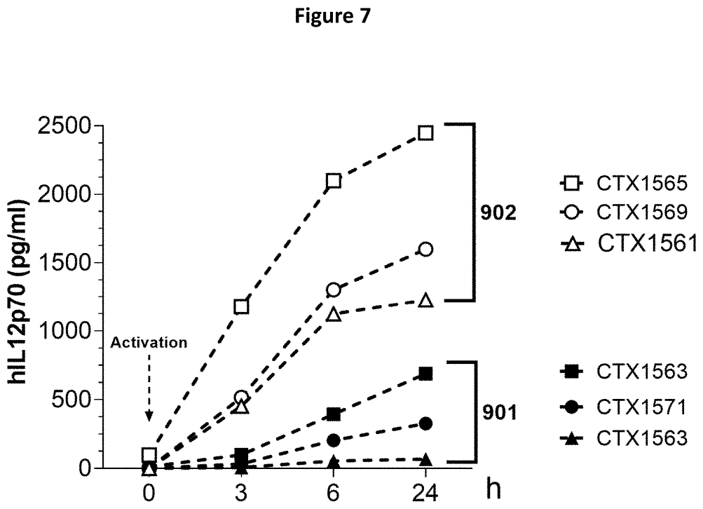

About one week post-electroporation, T cells were either left untreated (resting) or were treated with phorbol myristate acetate (PMA)/ionomycin for 0, 3, 6 and 24 hours to chemically active the cells. Next, supernatants were collected for to assess the amount of IL12 secretion from edited cells at each time point of stimulation using an ELISA for hIL12. As shown in FIGS. 4A, 4B, 5A, 5B, 6A, and 6B, unedited cells (Mock) did not secrete any IL12 regardless of which hIL12 splice variant (901 or 902) was used. However, edited T cells showed activation-dependent IL12 secretion in the supernatant over time when an IL12 expression construct was inserted into any of the three loci—the AAVS1 locus (FIGS. 4A and 4B), the CD70 locus (FIGS. 5A and 5B), and the B2M locus (FIGS. 6A and 6B). At any of the three loci, the constructs containing the hIL12 902 sequence and 3XAP1 binding site (FIGS. 4A, 5A, and 6A) provided a higher level of IL12 secretion following PMA activation compared to secretion levels of constructs that contained the hIL12 901 sequence (FIGS. 4B, 5B, and 6B). FIG. 7 demonstrates the difference in IL12 secretion from edited cells between constructs containing either hIL12 901 or hIL12 902 sequences with the 3xAP1 binding site. Overall, the highest amount of IL12 secretion was gained from cells containing the CTX1565 construct which included B2M homology arms allowing for insertion of IL12 into the B2M locus (FIG. 7).

### Example 4: Generation of CD70 CAR T that Conditionally Expressed hIL12 Only Upon Engagement with a Target Antigen

Allogeneic human T cells that lack expression of the TRAC gene, β2M gene, CD70 gene and AAVS1 gene, and express a chimeric antigen receptor (CAR) targeting CD70 were produced. The resulting edited CAR T cells also contained activation-dependent constructs that secrete hIL12 upon T cell activation. Specifically, the isolated human T cells were subjected to the electroporation method described in Example 1 to introduce two recombinant adeno-associated adenoviral vectors, serotype 6 (AAV6) (MOI 50, 000), and Cas9:sgRNA RNPs (1 μM Cas9, 5 μM gRNA).

One recombinant AAV contained the nucleotide sequence of SEQ ID NO: 21 (encoding anti-CD70 CAR comprising the amino acid sequence of SEQ ID NO: 19) and a second recombinant AAV contained one of the activation-dependent constructs described in Example 3 (CTX1560-CTX1571). In addition, the following sgRNAs were also used: TRAC (SEQ ID NO: 41), β2M (SEQ ID NO: 41), CD70 (SEQ ID NO: 59) and AAVS1 (SEQ ID NO: 33).

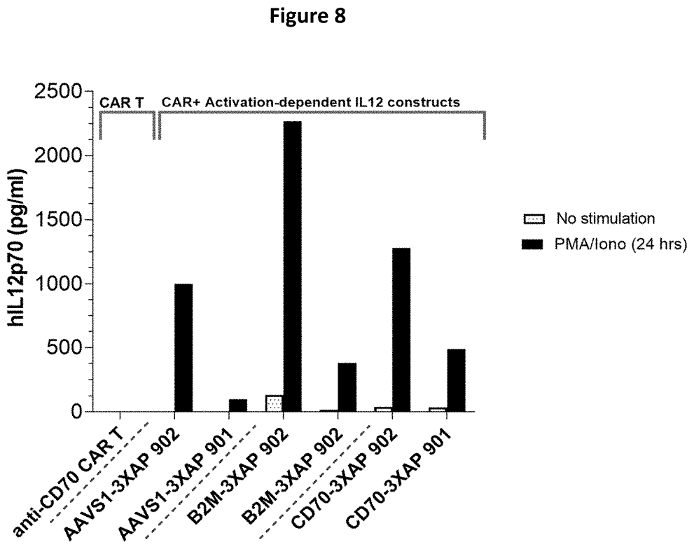

About one week post-electroporation, T cells were either left untreated or pharmacologically-stimulated with phorbol myristate acetate (PMA)/ionomycin for 24 hours. Then, supernatants were collected to assess IL12 secretion from edited cells using an ELISA for hIL12. As shown in FIG. 8, unedited cells (mock) did not secrete any IL12; however, edited T cells showed activation-dependent IL12 secretion. The highest level of IL12 secretion was gained from cells with the CTX1565 construct which contained B2M homology arms. Constructs containing CD70 homology arms yielded slightly higher amounts of IL12 secretion than constructs that contained AVVS1 homology arms; however, constructs that contained AVVS1 homology arms had no basal secretion unlike constructs containing CD70 homology arms. FIG. 8.

In another study, electroporated T cells were activated by CD70 antigen expressing cells instead of PMA/ionomycin week post electroporation. Specifically, A498 cells or 786-0 cells expressing CD70 target antigen were co-cultured overnight with edited anti-CD70 CAR T cells containing one of the activation-dependent constructs described in Example 3 (CTX1560-CTX1571). Supernatants were collected the next day to assess IL12 secretion from the edited CAR T cells using an ELISA for hIL12.

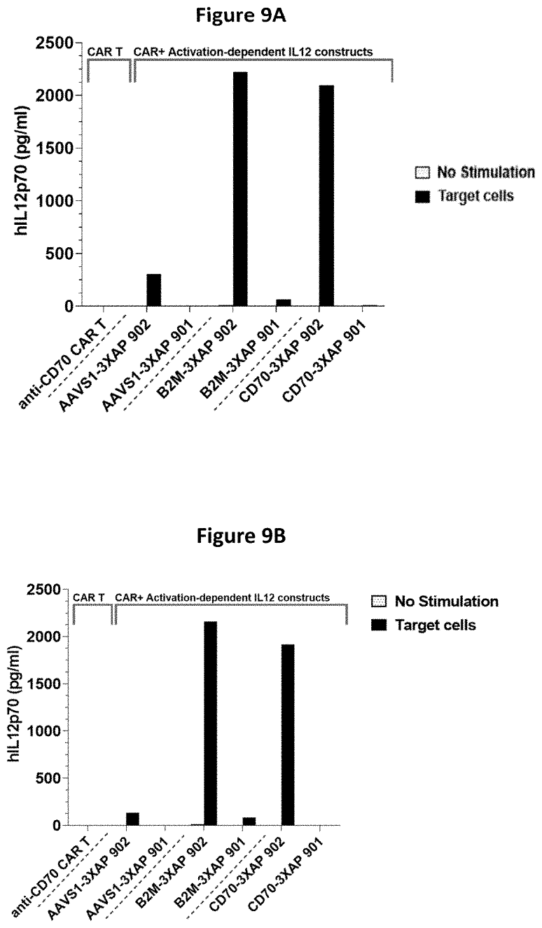

As shown in FIGS. 9A and 9B, only edited CAR T cells secreted IL12 upon CD70 CAR T stimulation with A498 (FIG. 9A) or 786-0 (FIG. 9B). IL12 expression was greatest from cells containing the CTX1565 construct which includes the 3XAP-IL12 902 expression construct inserted into the B2M locus.

As such, the data show that T cells were engineered to express transgenes only after either T cell activation or CAR molecule engagement with target tumor expressed antigen. Accordingly, data suggest that by controlling transgene expression by this manner can allow for spatial and temporal control of anti-tumor promoting factors, which may increase efficacy and safety of cellular therapies.

TABLE 7
Sequences of IL12-Containing Constructs

| Construct | SEQ ID | |
|---|---|---|
| ID | NO: | SEQUENCE |
| CTX936* | 74 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTCATTTT |
| | | GACACCCCCATAATATTTTTCCAGAATTAACAGTATAAATTGCATCTCTTGTTCA |
| | | AGAGTTCCCTATCACTCTCTTTAATCACTACTCACAGTAACCTCAACTCCTGCAA |
| | | GGCCAGCCCAGCACCAGCACCAGCCAACTCTCACTGAAGCCAGCTCTCTCTTCCT |
| | | CCACCACCATGGTGAGCAAGGGCGAGGAGCTGTTCACCGGGGTGGTGCCCATCCT |
| | | GGTCGAGCTGGACGGCGACGTAAACGGCCACAAGTTCAGCGTGTCCGGCGAGGGC |
| | | GAGGGCGATGCCACCTACGGCAAGCTGACCCTGAAGTTCATCTGCACCACCGGCA |
| | | AGCTGCCCGTGCCCTGGCCCACCCTCGTGACCACCCTGACCTACGGCGTGCAGTG |
| | | CTTCAGCCGCTACCCCGACCACATGAAGCAGCACGACTTCTTCAAGTCCGCCATG |
| | | CCCGAAGGCTACGTCCAGGAGCGCACCATCTTCTTCAAGGACGACGGCAACTACA |
| | | AGACCCGCGCCGAGGTGAAGTTCGAGGGCGACACCCTGGTGAACCGCATCGAGCT |
| | | GAAGGGCATCGACTTCAAGGAGGACGGCAACATCCTGGGGCACAAGCTGGAGTAC |
| | | AACTACAACAGCCACAACGTCTATATCATGGCCGACAAGCAGAAGAACGGCATCA |
| | | AGGTGAACTTCAAGATCCGCCACAACATCGAGGACGGCAGCGTGCAGCTCGCCGA |
| | | CCACTACCAGCAGAACACCCCCATCGGCGACGGCCCCGTGCTGCTGCCCGACAAC |
| | | CACTACCTGAGCACCCAGTCCGCCCTGAGCAAAGACCCCAACGAGAAGCGCGATC |
| | | ACATGGTCCTGCTGGAGTTCGTGACCGCCGCCGGGATCACTCTCGGCATGGACGA |
| | | GCTGTACAAGTCCGGACTCAGATCTCTCATGAAGCAGATCCAGAGCCATGGCTTC |
| | | CCGCCGGAGGTGGAGGAGCAGGATGATGGCACGCTGCCCATGTCTTGTGCCCAGG |
| | | AGAGCGGGATGGACCGTCACCCTGCAGCCTGTGCTTCTGCTAGGATCAATGTGTA |
| | | GAATAAAATCGCTATCCATCGAAGATGGATGTGTGTTGGTTTTTTGTGTGACTGT |
| | | GGGGTGGAGGGGACAGATAAAAGTACCCAGAACCAGAGCCACATTAACCGGCCCT |
| | | GGGAATATAAGGTGGTCCCAGCTCGGGGACACAGGATCCCTGGAGGCAGCAAACA |
| | | TGCTGTCCTGAAGTGGACATAGGGGCCCGGGTTGGAGGAAGAAGACTAGCTGAGC |
| | | TCTCGGACCCCTGGAAGATGCCATGACAGGGGGCTGGAAGAGCTAGCACAGACTA |
| | | GAGAGGTAAGGGGGGTAGGGGAGCTGCCCAAATGAAAGGAGTGAGAGGTGACCCG |
| | | AATCCACAGGAGAACGGGGTGTCCAGGCAAAGAAAGCAAGAGGATGGAGAGGTGG |
| | | CTAAAGCCAGGGAGACGGGGTACTTTGGGGTTGTCCAGAAAAACGGTGATGATGC |
| | | AGGCCTACAAGAAGGGGAGGCGGGACGCAAGGGAGACATCCGTCGGAGAAGGCCA |
| | | TCCTAAGAAACGAGAGATGGCACAGGCCCCAGAAGGAGAAGGAAAAGGGAACCCA |
| | | GCGAGTGAAGACGGCATGGGGTTGGGTGAGGGAGGAGAGATGCCCGGAGAGGACC |
| | | CAGACACGGGGAGGATCCGCTCAGAGGACATCACGTGGTGCAGCGCCGAGAAGGA |
| | | AGTGCTCCGGAAAGAGCATCCTTGGGCAGCAACACAGCAGAGAGCAAGGGGAAGA |
| | | GGGAGTGGAGGAAGACGGAACCTGAAGGAGGCGGC |
| CTX937 | 75 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTTCCCAT |
| | | CCTCGAGGCTGGGGATTCCCCATCTCGAGGCTGGGGATTCCCCATCTCGAGGCTG |
| | | GGGATTCCCCATCTCGACCGTCCATCCATCATTTTGACACCCCCATAATATTTTT |
| | | CCAGAATTAACAGTATAAATTGCATCTCTTGTTCAAGAGTTCCCTATCACTCTCT |
| | | TTAATCACTACTCACAGTAACCTCAACTCCTGCAAGGCCAGCCCAGCACCAGCAC |
| | | CAGCCAACTCTCACTGAAGCCAGCTCTCTCTTCCTCCACCACCATGGTGAGCAAG |
| | | GGCGAGGAGCTGTTCACCGGGGTGGTGCCCATCCTGGTCGAGCTGGACGGCGACG |
| | | TAAACGGCCACAAGTTCAGCGTGTCCGGCGAGGGCGAGGGCGATGCCACCTACGG |
| | | CAAGCTGACCCTGAAGTTCATCTGCACCACCGGCAAGCTGCCCGTGCCCTGGCCC |
| | | ACCCTCGTGACCACCCTGACCTACGGCGTGCAGTGCTTCAGCCGCTACCCCGACC |
| | | ACATGAAGCAGCACGACTTCTTCAAGTCCGCCATGCCCGAAGGCTACGTCCAGGA |
| | | GCGCACCATCTTCTTCAAGGACGACGGCAACTACAAGACCCGCGCCGAGGTGAAG |
| | | TTCGAGGGCGACACCCTGGTGAACCGCATCGAGCTGAAGGGCATCGACTTCAAGG |
| | | AGGACGGCAACATCCTGGGGCACAAGCTGGAGTACAACTACAACAGCCACAACGT |
| | | CTATATCATGGCCGACAAGCAGAAGAACGGCATCAAGGTGAACTTCAAGATCCGC |
| | | CACAACATCGAGGACGGCAGCGTGCAGCTCGCCGACCACTACCAGCAGAACACCC |
| | | CCATCGGCGACGGCCCCGTGCTGCTGCCCGACAACCACTACCTGAGCACCCAGTC |
| | | CGCCCTGAGCAAAGACCCCAACGAGAAGCGCGATCACATGGTCCTGCTGGAGTTC |
| | | GTGACCGCCGCCGGGATCACTCTCGGCATGGACGAGCTGTACAAGTCCGGACTCA |
| | | GATCTCTCATGAAGCAGATCCAGAGCCATGGCTTCCCGCCGGAGGTGGAGGAGCA |
| | | GGATGATGGCACGCTGCCCATGTCTTGTGCCCAGGAGAGCGGGATGGACCGTCAC |
| | | CCTGCAGCCTGTGCTTCTGCTAGGATCAATGTGTAGAATAAAATCGCTATCCATC |
| | | GAAGATGGATGTGTGTTGGTTTTTTGTGTGACTGTGGGGTGGAGGGGACAGATAA |
| | | AAGTACCCAGAACCAGAGCCACATTAACCGGCCCTGGGAATATAAGGTGGTCCCA |
| | | GCTCGGGGACACAGGATCCCTGGAGGCAGCAAACATGCTGTCCTGAAGTGGACAT |
| | | AGGGGCCCGGGTTGGAGGAAGAAGACTAGCTGAGCTCTCGGACCCCTGGAAGATG |
| | | CCATGACAGGGGGCTGGAAGAGCTAGCACAGACTAGAGAGGTAAGGGGGGTAGGG |
| | | GAGCTGCCCAAATGAAAGGAGTGAGAGGTGACCCGAATCCACAGGAGAACGGGGT |
| | | GTCCAGGCAAAGAAAGCAAGAGGATGGAGAGGTGGCTAAAGCCAGGGAGACGGGG |
| | | TACTTTGGGGTTGTCCAGAAAAACGGTGATGATGCAGGCCTACAAGAAGGGGAGG |
| | | CGGGACGCAAGGGAGACATCCGTCGGAGAAGGCCATCCTAAGAAACGAGAGATGG |
| | | CACAGGCCCCAGAAGGAGAAGGAAAAGGGAACCCAGCGAGTGAAGACGGCATGGG |
| | | GTTGGGTGAGGGAGGAGAGATGCCCGGAGAGGACCCAGACACGGGGAGGATCCGC |
| | | TCAGAGGACATCACGTGGTGCAGCGCCGAGAAGGAAGTGCTCCGGAAAGAGCATC |
| | | CTTGGGCAGCAACACAGCAGAGAGCAAGGGGAAGAGGGAGTGGAGGAAGACGGAA |
| | | CCTGAAGGAGGCGGC |
| CTX938 | 76 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTCTTACG |
| | | CGTGCTAGCCCCGATGTTTTCTGAGTTACTTTTGTATCCCCACCCCCCCTCGAGG |
| | | AGGAAAAACTGTTTCATACAGAAGGCGTACGCCTTCTGTATGAAACAGTTTTTCC |
| | | TCCACGCCTTCTGTATGAAACAGTTTTTCCTCCTCGAGGACATTTTGACACCCCC |
| | | ATAATATTTTTCCAGAATTAACAGTATAAATTGCATCTCTTGTTCAAGAGTTCCC |
| | | TATCACTCTCTTTAATCACTACTCACAGTAACCTCAACTCCTGCAAGGCCAGCCC |
| | | AGCACCAGCACCAGCCAACTCTCACTGAAGCCAGCTCTCTCTTCCTCCACCACCA |
| | | TGGTGAGCAAGGGCGAGGAGCTGTTCACCGGGGTGGTGCCCATCCTGGTCGAGCT |
| | | GGACGGCGACGTAAACGGCCACAAGTTCAGCGTGTCCGGCGAGGGCGAGGGCGAT |
| | | GCCACCTACGGCAAGCTGACCCTGAAGTTCATCTGCACCACCGGCAAGCTGCCCG |
| | | TGCCCTGGCCCACCCTCGTGACCACCCTGACCTACGGCGTGCAGTGCTTCAGCCG |
| | | CTACCCCGACCACATGAAGCAGCACGACTTCTTCAAGTCCGCCATGCCCGAAGGC |
| | | TACGTCCAGGAGCGCACCATCTTCTTCAAGGACGACGGCAACTACAAGACCCGCG |
| | | CCGAGGTGAAGTTCGAGGGCGACACCCTGGTGAACCGCATCGAGCTGAAGGGCAT |
| | | CGACTTCAAGGAGGACGGCAACATCCTGGGGCACAAGCTGGAGTACAACTACAAC |
| | | AGCCACAACGTCTATATCATGGCCGACAAGCAGAAGAACGGCATCAAGGTGAACT |
| | | TCAAGATCCGCCACAACATCGAGGACGGCAGCGTGCAGCTCGCCGACCACTACCA |
| | | GCAGAACACCCCCATCGGCGACGGCCCCGTGCTGCTGCCCGACAACCACTACCTG |
| | | AGCACCCAGTCCGCCCTGAGCAAAGACCCCAACGAGAAGCGCGATCACATGGTCC |
| | | TGCTGGAGTTCGTGACCGCCGCCGGGATCACTCTCGGCATGGACGAGCTGTACAA |
| | | GTCCGGACTCAGATCTCTCATGAAGCAGATCCAGAGCCATGGCTTCCCGCCGGAG |
| | | GTGGAGGAGCAGGATGATGGCACGCTGCCCATGTCTTGTGCCCAGGAGAGCGGGA |
| | | TGGACCGTCACCCTGCAGCCTGTGCTTCTGCTAGGATCAATGTGTAGAATAAAAT |
| | | CGCTATCCATCGAAGATGGATGTGTGTTGGTTTTTTGTGTGACTGTGGGGTGGAG |
| | | GGGACAGATAAAAGTACCCAGAACCAGAGCCACATTAACCGGCCCTGGGAATATA |
| | | AGGTGGTCCCAGCTCGGGGACACAGGATCCCTGGAGGCAGCAAACATGCTGTCCT |
| | | GAAGTGGACATAGGGGCCCGGGTTGGAGGAAGAAGACTAGCTGAGCTCTCGGACC |
| | | CCTGGAAGATGCCATGACAGGGGGCTGGAAGAGCTAGCACAGACTAGAGAGGTAA |
| | | GGGGGGTAGGGGAGCTGCCCAAATGAAAGGAGTGAGAGGTGACCCGAATCCACAG |
| | | GAGAACGGGGTGTCCAGGCAAAGAAAGCAAGAGGATGGAGAGGTGGCTAAAGCCA |
| | | GGGAGACGGGGTACTTTGGGGTTGTCCAGAAAAACGGTGATGATGCAGGCCTACA |
| | | AGAAGGGGAGGCGGGACGCAAGGGAGACATCCGTCGGAGAAGGCCATCCTAAGAA |
| | | ACGAGAGATGGCACAGGCCCCAGAAGGAGAAGGAAAAGGGAACCCAGCGAGTGAA |
| | | GACGGCATGGGGTTGGGTGAGGGAGGAGAGATGCCCGGAGAGGACCCAGACACGG |
| | | GGAGGATCCGCTCAGAGGACATCACGTGGTGCAGCGCCGAGAAGGAAGTGCTCCG |
| | | GAAAGAGCATCCTTGGGCAGCAACACAGCAGAGAGCAAGGGGAAGAGGGAGTGGA |
| | | GGAAGACGGAACCTGAAGGAGGCGGC |
| CTX939 | 77 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTTACGCG |
| | | TGCTAGCCCGGGCACTGACTCATCAAGCACTGACTCATCAAGCACTGACTCATCA |
| | | AGGGACTCAGGGAGGGAAACTCCATTTTGACACCCCCATAATATTTTTCCAGAAT |
| | | TAACAGTATAAATTGCATCTCTTGTTCAAGAGTTCCCTATCACTCTCTTTAATCA |
| | | CTACTCACAGTAACCTCAACTCCTGCAAGGCCAGCCCAGCACCAGCACCAGCCAA |
| | | CTCTCACTGAAGCCAGCTCTCTCTTCCTCCACCACCATGGTGAGCAAGGGCGAGG |
| | | AGCTGTTCACCGGGGTGGTGCCCATCCTGGTCGAGCTGGACGGCGACGTAAACGG |
| | | CCACAAGTTCAGCGTGTCCGGCGAGGGCGAGGGCGATGCCACCTACGGCAAGCTG |
| | | ACCCTGAAGTTCATCTGCACCACCGGCAAGCTGCCCGTGCCCTGGCCCACCCTCG |
| | | TGACCACCCTGACCTACGGCGTGCAGTGCTTCAGCCGCTACCCCGACCACATGAA |
| | | GCAGCACGACTTCTTCAAGTCCGCCATGCCCGAAGGCTACGTCCAGGAGCGCACC |
| | | ATCTTCTTCAAGGACGACGGCAACTACAAGACCCGCGCCGAGGTGAAGTTCGAGG |
| | | GCGACACCCTGGTGAACCGCATCGAGCTGAAGGGCATCGACTTCAAGGAGGACGG |
| | | CAACATCCTGGGGCACAAGCTGGAGTACAACTACAACAGCCACAACGTCTATATC |
| | | ATGGCCGACAAGCAGAAGAACGGCATCAAGGTGAACTTCAAGATCCGCCACAACA |
| | | TCGAGGACGGCAGCGTGCAGCTCGCCGACCACTACCAGCAGAACACCCCCATCGG |
| | | CGACGGCCCCGTGCTGCTGCCCGACAACCACTACCTGAGCACCCAGTCCGCCCTG |
| | | AGCAAAGACCCCAACGAGAAGCGCGATCACATGGTCCTGCTGGAGTTCGTGACCG |
| | | CCGCCGGGATCACTCTCGGCATGGACGAGCTGTACAAGTCCGGACTCAGATCTCT |
| | | CATGAAGCAGATCCAGAGCCATGGCTTCCCGCCGGAGGTGGAGGAGCAGGATGAT |
| | | GGCACGCTGCCCATGTCTTGTGCCCAGGAGAGCGGGATGGACCGTCACCCTGCAG |
| | | CCTGTGCTTCTGCTAGGATCAATGTGTAGAATAAAATCGCTATCCATCGAAGATG |
| | | GATGTGTGTTGGTTTTTTGTGTGACTGTGGGGTGGAGGGGACAGATAAAAGTACC |
| | | CAGAACCAGAGCCACATTAACCGGCCCTGGGAATATAAGGTGGTCCCAGCTCGGG |
| | | GACACAGGATCCCTGGAGGCAGCAAACATGCTGTCCTGAAGTGGACATAGGGGCC |
| | | CGGGTTGGAGGAAGAAGACTAGCTGAGCTCTCGGACCCCTGGAAGATGCCATGAC |
| | | AGGGGGCTGGAAGAGCTAGCACAGACTAGAGAGGTAAGGGGGGTAGGGGAGCTGC |
| | | CCAAATGAAAGGAGTGAGAGGTGACCCGAATCCACAGGAGAACGGGGTGTCCAGG |
| | | CAAAGAAAGCAAGAGGATGGAGAGGTGGCTAAAGCCAGGGAGACGGGGTACTTTG |
| | | GGGTTGTCCAGAAAAACGGTGATGATGCAGGCCTACAAGAAGGGGAGGCGGGACG |
| | | CAAGGGAGACATCCGTCGGAGAAGGCCATCCTAAGAAACGAGAGATGGCACAGGC |
| | | CCCAGAAGGAGAAGGAAAAGGGAACCCAGCGAGTGAAGACGGCATGGGGTTGGGT |
| | | GAGGGAGGAGAGATGCCCGGAGAGGACCCAGACACGGGGAGGATCCGCTCAGAGG |
| | | ACATCACGTGGTGCAGCGCCGAGAAGGAAGTGCTCCGGAAAGAGCATCCTTGGGC |
| | | AGCAACACAGCAGAGAGCAAGGGGAAGAGGGAGTGGAGGAAGACGGAACCTGAAG |
| | | GAGGCGGC |
| CTX940 | 78 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTGCCTAA |
| | | CTGGCCGGTACCTGAGCTCAGTTCTGAGAAAAGTAGTTCTGAGAAAAGTAGTTCT |
| | | GAGAAAAGTAGTTCTGAGAAAAGTAGTTCTGAGAAAAGTCCATTTTGACACCCCC |
| | | ATAATATTTTTCCAGAATTAACAGTATAAATTGCATCTCTTGTTCAAGAGTTCCC |
| | | TATCACTCTCTTTAATCACTACTCACAGTAACCTCAACTCCTGCAAGGCCAGCCC |
| | | AGCACCAGCACCAGCCAACTCTCACTGAAGCCAGCTCTCTCTTCCTCCACCACCA |
| | | TGGTGAGCAAGGGCGAGGAGCTGTTCACCGGGGTGGTGCCCATCCTGGTCGAGCT |
| | | GGACGGCGACGTAAACGGCCACAAGTTCAGCGTGTCCGGCGAGGGCGAGGGCGAT |
| | | GCCACCTACGGCAAGCTGACCCTGAAGTTCATCTGCACCACCGGCAAGCTGCCCG |
| | | TGCCCTGGCCCACCCTCGTGACCACCCTGACCTACGGCGTGCAGTGCTTCAGCCG |
| | | CTACCCCGACCACATGAAGCAGCACGACTTCTTCAAGTCCGCCATGCCCGAAGGC |
| | | TACGTCCAGGAGCGCACCATCTTCTTCAAGGACGACGGCAACTACAAGACCCGCG |
| | | CCGAGGTGAAGTTCGAGGGCGACACCCTGGTGAACCGCATCGAGCTGAAGGGCAT |
| | | CGACTTCAAGGAGGACGGCAACATCCTGGGGCACAAGCTGGAGTACAACTACAAC |
| | | AGCCACAACGTCTATATCATGGCCGACAAGCAGAAGAACGGCATCAAGGTGAACT |
| | | TCAAGATCCGCCACAACATCGAGGACGGCAGCGTGCAGCTCGCCGACCACTACCA |
| | | GCAGAACACCCCCATCGGCGACGGCCCCGTGCTGCTGCCCGACAACCACTACCTG |
| | | AGCACCCAGTCCGCCCTGAGCAAAGACCCCAACGAGAAGCGCGATCACATGGTCC |
| | | TGCTGGAGTTCGTGACCGCCGCCGGGATCACTCTCGGCATGGACGAGCTGTACAA |
| | | GTCCGGACTCAGATCTCTCATGAAGCAGATCCAGAGCCATGGCTTCCCGCCGGAG |
| | | GTGGAGGAGCAGGATGATGGCACGCTGCCCATGTCTTGTGCCCAGGAGAGCGGGA |
| | | TGGACCGTCACCCTGCAGCCTGTGCTTCTGCTAGGATCAATGTGTAGAATAAAAT |
| | | CGCTATCCATCGAAGATGGATGTGTGTTGGTTTTTTGTGTGACTGTGGGGTGGAG |
| | | GGGACAGATAAAAGTACCCAGAACCAGAGCCACATTAACCGGCCCTGGGAATATA |
| | | AGGTGGTCCCAGCTCGGGGACACAGGATCCCTGGAGGCAGCAAACATGCTGTCCT |
| | | GAAGTGGACATAGGGGCCCGGGTTGGAGGAAGAAGACTAGCTGAGCTCTCGGACC |
| | | CCTGGAAGATGCCATGACAGGGGGCTGGAAGAGCTAGCACAGACTAGAGAGGTAA |
| | | GGGGGGTAGGGGAGCTGCCCAAATGAAAGGAGTGAGAGGTGACCCGAATCCACAG |
| | | GAGAACGGGGTGTCCAGGCAAAGAAAGCAAGAGGATGGAGAGGTGGCTAAAGCCA |
| | | GGGAGACGGGGTACTTTGGGGTTGTCCAGAAAAACGGTGATGATGCAGGCCTACA |
| | | AGAAGGGGAGGCGGGACGCAAGGGAGACATCCGTCGGAGAAGGCCATCCTAAGAA |
| | | ACGAGAGATGGCACAGGCCCCAGAAGGAGAAGGAAAAGGGAACCCAGCGAGTGAA |
| | | GACGGCATGGGGTTGGGTGAGGGAGGAGAGATGCCCGGAGAGGACCCAGACACGG |
| | | GGAGGATCCGCTCAGAGGACATCACGTGGTGCAGCGCCGAGAAGGAAGTGCTCCG |
| | | GAAAGAGCATCCTTGGGCAGCAACACAGCAGAGAGCAAGGGGAAGAGGGAGTGGA |
| | | GGAAGACGGAACCTGAAGGAGGCGGC |
| CTX941 | 79 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTATCGAT |
| | | AGGTACTAAGTCTAGACGGCAGTCTAGACGTACTAAGTCTAGACGGCAGTCTAGA |
| | | CGTACCGAGCTCTTACGCGTGCTAGCCCGGGCTCGAGATCTGCGATCCATTTTGA |
| | | CACCCCCATAATATTTTTCCAGAATTAACAGTATAAATTGCATCTCTTGTTCAAG |
| | | AGTTCCCTATCACTCTCTTTAATCACTACTCACAGTAACCTCAACTCCTGCAAGG |
| | | CCAGCCCAGCACCAGCACCAGCCAACTCTCACTGAAGCCAGCTCTCTCTTCCTCC |
| | | ACCACCATGGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGA |
| | | GGGCGATGCCACCTACGGCAAGCTGACCCTGAAGTTCATCTGCACCACCGGCAAG |
| | | CTGCCCGTGCCCTGGCCCACCCTCGTGACCACCCTGACCTACGGCGTGCAGTGCT |
| | | TCAGCCGCTACCCCGACCACATGAAGCAGCACGACTTCTTCAAGTCCGCCATGCC |
| | | CGAAGGCTACGTCCAGGAGCGCACCATCTTCTTCAAGGACGACGGCAACTACAAG |
| | | ACCCGCGCCGAGGTGAAGTTCGAGGGCGACACCCTGGTGAACCGCATCGAGCTGA |
| | | AGGGCATCGACTTCAAGGAGGACGGCAACATCCTGGGGCACAAGCTGGAGTACAA |
| | | CTACAACAGCCACAACGTCTATATCATGGCCGACAAGCAGAAGAACGGCATCAAG |
| | | GTGAACTTCAAGATCCGCCACAACATCGAGGACGGCAGCGTGCAGCTCGCCGACC |
| | | ACTACCAGCAGAACACCCCCATCGGCGACGGCCCCGTGCTGCTGCCCGACAACCA |
| | | CTACCTGAGCACCCAGTCCGCCCTGAGCAAAGACCCCAACGAGAAGCGCGATCAC |
| | | ATGGTCCTGCTGGAGTTCGTGACCGCCGCCGGGATCACTCTCGGCATGGACGAGC |
| | | TGTACAAGTCCGGACTCAGATCTCTCATGAAGCAGATCCAGAGCCATGGCTTCCC |
| | | GCCGGAGGTGGAGGAGCAGGATGATGGCACGCTGCCCATGTCTTGTGCCCAGGAG |
| | | AGCGGGATGGACCGTCACCCTGCAGCCTGTGCTTCTGCTAGGATCAATGTGTAGA |
| | | ATAAAATCGCTATCCATCGAAGATGGATGTGTGTTGGTTTTTTGTGTGACTGTGG |
| | | GGTGGAGGGGACAGATAAAAGTACCCAGAACCAGAGCCACATTAACCGGCCCTGG |
| | | GAATATAAGGTGGTCCCAGCTCGGGGACACAGGATCCCTGGAGGCAGCAAACATG |
| | | CTGTCCTGAAGTGGACATAGGGGCCCGGGTTGGAGGAAGAAGACTAGCTGAGCTC |
| | | TCGGACCCCTGGAAGATGCCATGACAGGGGGCTGGAAGAGCTAGCACAGACTAGA |
| | | GAGGTAAGGGGGGTAGGGGAGCTGCCCAAATGAAAGGAGTGAGAGGTGACCCGAA |
| | | TCCACAGGAGAACGGGGTGTCCAGGCAAAGAAAGCAAGAGGATGGAGAGGTGGCT |
| | | AAAGCCAGGGAGACGGGGTACTTTGGGGTTGTCCAGAAAAACGGTGATGATGCAG |
| | | GCCTACAAGAAGGGGAGGCGGGACGCAAGGGAGACATCCGTCGGAGAAGGCCATC |
| | | CTAAGAAACGAGAGATGGCACAGGCCCCAGAAGGAGAAGGAAAAGGGAACCCAGC |
| | | GAGTGAAGACGGCATGGGGTTGGGTGAGGGAGGAGAGATGCCCGGAGAGGACCCA |
| | | GACACGGGGAGGATCCGCTCAGAGGACATCACGTGGTGCAGCGCCGAGAAGGAAG |
| | | TGCTCCGGAAAGAGCATCCTTGGGCAGCAACACAGCAGAGAGCAAGGGGAAGAGG |
| | | GAGTGGAGGAAGACGGAACCTGAAGGAGGCGGC |
| CTX942* | 80 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTAGACGC |
| | | TAGCGGGGGGCTATAAAAGGGGGTGGGGGCGTTCGTCCTCACTCTAAGGCCAGCC |
| | | CAGCACCAGCACCAGCCAACTCTCACTGAAGCCAGCTCTCTCTTCCTCCACCACC |
| | | ATGGTGAGCAAGGGCGAGGAGCTGTTCACCGGGGTGGTGCCCATCCTGGTCGAGC |
| | | TGGACGGCGACGTAAACGGCCACAAGTTCAGCGTGTCCGGCGAGGGCGAGGGCGA |
| | | TGCCACCTACGGCAAGCTGACCCTGAAGTTCATCTGCACCACCGGCAAGCTGCCC |
| | | GTGCCCTGGCCCACCCTCGTGACCACCCTGACCTACGGCGTGCAGTGCTTCAGCC |
| | | GCTACCCCGACCACATGAAGCAGCACGACTTCTTCAAGTCCGCCATGCCCGAAGG |
| | | CTACGTCCAGGAGCGCACCATCTTCTTCAAGGACGACGGCAACTACAAGACCCGC |
| | | GCCGAGGTGAAGTTCGAGGGCGACACCCTGGTGAACCGCATCGAGCTGAAGGGCA |
| | | TCGACTTCAAGGAGGACGGCAACATCCTGGGGCACAAGCTGGAGTACAACTACAA |
| | | CAGCCACAACGTCTATATCATGGCCGACAAGCAGAAGAACGGCATCAAGGTGAAC |
| | | TTCAAGATCCGCCACAACATCGAGGACGGCAGCGTGCAGCTCGCCGACCACTACC |
| | | AGCAGAACACCCCCATCGGCGACGGCCCCGTGCTGCTGCCCGACAACCACTACCT |
| | | GAGCACCCAGTCCGCCCTGAGCAAAGACCCCAACGAGAAGCGCGATCACATGGTC |
| | | CTGCTGGAGTTCGTGACCGCCGCCGGGATCACTCTCGGCATGGACGAGCTGTACA |
| | | AGTCCGGACTCAGATCTCTCATGAAGCAGATCCAGAGCCATGGCTTCCCGCCGGA |
| | | GGTGGAGGAGCAGGATGATGGCACGCTGCCCATGTCTTGTGCCCAGGAGAGCGGG |
| | | ATGGACCGTCACCCTGCAGCCTGTGCTTCTGCTAGGATCAATGTGTAGAATAAAA |
| | | TCGCTATCCATCGAAGATGGATGTGTGTTGGTTTTTTGTGTGACTGTGGGGTGGA |
| | | GGGGACAGATAAAAGTACCCAGAACCAGAGCCACATTAACCGGCCCTGGGAATAT |
| | | AAGGTGGTCCCAGCTCGGGGACACAGGATCCCTGGAGGCAGCAAACATGCTGTCC |
| | | TGAAGTGGACATAGGGGCCCGGGTTGGAGGAAGAAGACTAGCTGAGCTCTCGGAC |
| | | CCCTGGAAGATGCCATGACAGGGGGCTGGAAGAGCTAGCACAGACTAGAGAGGTA |
| | | AGGGGGGTAGGGGAGCTGCCCAAATGAAAGGAGTGAGAGGTGACCCGAATCCACA |
| | | GGAGAACGGGGTGTCCAGGCAAAGAAAGCAAGAGGATGGAGAGGTGGCTAAAGCC |
| | | AGGGAGACGGGGTACTTTGGGGTTGTCCAGAAAAACGGTGATGATGCAGGCCTAC |
| | | AAGAAGGGGAGGCGGGACGCAAGGGAGACATCCGTCGGAGAAGGCCATCCTAAGA |
| | | AACGAGAGATGGCACAGGCCCCAGAAGGAGAAGGAAAAGGGAACCCAGCGAGTGA |
| | | AGACGGCATGGGGTTGGGTGAGGGAGGAGAGATGCCCGGAGAGGACCCAGACACG |
| | | GGGAGGATCCGCTCAGAGGACATCACGTGGTGCAGCGCCGAGAAGGAAGTGCTCC |
| | | GGAAAGAGCATCCTTGGGCAGCAACACAGCAGAGAGCAAGGGGAAGAGGGAGTGG |
| | | AGGAAGACGGAACCTGAAGGAGGCGGC |
| CTX943 | 81 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTTCCCAT |
| | | CCTCGAGGCTGGGGATTCCCCATCTCGAGGCTGGGGATTCCCCATCTCGAGGCTG |
| | | GGGATTCCCCATCTCGACCGTCCATCCATAGACGCTAGCGGGGGGCTATAAAAGG |
| | | GGGTGGGGGCGTTCGTCCTCACTCTAAGGCCAGCCCAGCACCAGCACCAGCCAAC |
| | | TCTCACTGAAGCCAGCTCTCTCTTCCTCCACCACCATGGTGAGCAAGGGCGAGGA |
| | | GCTGTTCACCGGGGTGGTGCCCATCCTGGTCGAGCTGGACGGCGACGTAAACGGC |
| | | CACAAGTTCAGCGTGTCCGGCGAGGGCGAGGGCGATGCCACCTACGGCAAGCTGA |
| | | CCCTGAAGTTCATCTGCACCACCGGCAAGCTGCCCGTGCCCTGGCCCACCCTCGT |
| | | GACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCA |
| | | TCTTCTTCAAGGACGACGGCAACTACAAGACCCGCGCCGAGGTGAAGTTCGAGGG |
| | | CGACACCCTGGTGAACCGCATCGAGCTGAAGGGCATCGACTTCAAGGAGGACGGC |
| | | AACATCCTGGGGCACAAGCTGGAGTACAACTACAACAGCCACAACGTCTATATCA |
| | | TGGCCGACAAGCAGAAGAACGGCATCAAGGTGAACTTCAAGATCCGCCACAACAT |
| | | CGAGGACGGCAGCGTGCAGCTCGCCGACCACTACCAGCAGAACACCCCCATCGGC |
| | | GACGGCCCCGTGCTGCTGCCCGACAACCACTACCTGAGCACCCAGTCCGCCCTGA |
| | | GCAAAGACCCCAACGAGAAGCGCGATCACATGGTCCTGCTGGAGTTCGTGACCGC |
| | | CGCCGGGATCACTCTCGGCATGGACGAGCTGTACAAGTCCGGACTCAGATCTCTC |
| | | ATGAAGCAGATCCAGAGCCATGGCTTCCCGCCGGAGGTGGAGGAGCAGGATGATG |
| | | GCACGCTGCCCATGTCTTGTGCCCAGGAGAGCGGGATGGACCGTCACCCTGCAGC |
| | | CTGTGCTTCTGCTAGGATCAATGTGTAGAATAAAATCGCTATCCATCGAAGATGG |
| | | ATGTGTGTTGGTTTTTTGTGTGACTGTGGGGTGGAGGGGACAGATAAAAGTACCC |
| | | AGAACCAGAGCCACATTAACCGGCCCTGGGAATATAAGGTGGTCCCAGCTCGGGG |
| | | ACACAGGATCCCTGGAGGCAGCAAACATGCTGTCCTGAAGTGGACATAGGGGCCC |
| | | GGGTTGGAGGAAGAAGACTAGCTGAGCTCTCGGACCCCTGGAAGATGCCATGACA |
| | | GGGGGCTGGAAGAGCTAGCACAGACTAGAGAGGTAAGGGGGGTAGGGGAGCTGCC |
| | | CAAATGAAAGGAGTGAGAGGTGACCCGAATCCACAGGAGAACGGGGTGTCCAGGC |
| | | AAAGAAAGCAAGAGGATGGAGAGGTGGCTAAAGCCAGGGAGACGGGGTACTTTGG |
| | | GGTTGTCCAGAAAAACGGTGATGATGCAGGCCTACAAGAAGGGGAGGCGGGACGC |
| | | AAGGGAGACATCCGTCGGAGAAGGCCATCCTAAGAAACGAGAGATGGCACAGGCC |
| | | CCAGAAGGAGAAGGAAAAGGGAACCCAGCGAGTGAAGACGGCATGGGGTTGGGTG |
| | | AGGGAGGAGAGATGCCCGGAGAGGACCCAGACACGGGGAGGATCCGCTCAGAGGA |
| | | CATCACGTGGTGCAGCGCCGAGAAGGAAGTGCTCCGGAAAGAGCATCCTTGGGCA |
| | | GCAACACAGCAGAGAGCAAGGGGAAGAGGGAGTGGAGGAAGACGGAACCTGAAGG |
| | | AGGCGGC |
| CTX944 | 82 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTCTTACG |
| | | CGTGCTAGCCCCGATGTTTTCTGAGTTACTTTTGTATCCCCACCCCCCCTCGAGG |
| | | AGGAAAAACTGTTTCATACAGAAGGCGTACGCCTTCTGTATGAAACAGTTTTTCC |
| | | TCCACGCCTTCTGTATGAAACAGTTTTTCCTCCTCGAGGAAGACGCTAGCGGGGG |
| | | GCTATAAAAGGGGGTGGGGGCGTTCGTCCTCACTCTAAGGCCAGCCCAGCACCAG |
| | | CACCAGCCAACTCTCACTGAAGCCAGCTCTCTCTTCCTCCACCACCATGGTGAGC |
| | | AAGGGCGAGGAGCTGTTCACCGGGGTGGTGCCCATCCTGGTCGAGCTGGACGGCG |
| | | ACGTAAACGGCCACAAGTTCAGCGTGTCCGGCGAGGGCGAGGGCGATGCCACCTA |
| | | CGGCAAGCTGACCCTGAAGTTCATCTGCACCACCGGCAAGCTGCCCGTGCCCTGG |
| | | CCCACCCTCGTGACCACCCTGACCTACGGCGTGCAGTGCTTCAGCCGCTACCCCG |
| | | ACCACATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCCGAGGTG |
| | | AAGTTCGAGGGCGACACCCTGGTGAACCGCATCGAGCTGAAGGGCATCGACTTCA |
| | | AGGAGGACGGCAACATCCTGGGGCACAAGCTGGAGTACAACTACAACAGCCACAA |
| | | CGTCTATATCATGGCCGACAAGCAGAAGAACGGCATCAAGGTGAACTTCAAGATC |
| | | CGCCACAACATCGAGGACGGCAGCGTGCAGCTCGCCGACCACTACCAGCAGAACA |
| | | CCCCCATCGGCGACGGCCCCGTGCTGCTGCCCGACAACCACTACCTGAGCACCCA |
| | | GTCCGCCCTGAGCAAAGACCCCAACGAGAAGCGCGATCACATGGTCCTGCTGGAG |
| | | TTCGTGACCGCCGCCGGGATCACTCTCGGCATGGACGAGCTGTACAAGTCCGGAC |
| | | TCAGATCTCTCATGAAGCAGATCCAGAGCCATGGCTTCCCGCCGGAGGTGGAGGA |
| | | GCAGGATGATGGCACGCTGCCCATGTCTTGTGCCCAGGAGAGCGGGATGGACCGT |
| | | CACCCTGCAGCCTGTGCTTCTGCTAGGATCAATGTGTAGAATAAAATCGCTATCC |
| | | ATCGAAGATGGATGTGTGTTGGTTTTTTGTGTGACTGTGGGGTGGAGGGGACAGA |
| | | TAAAAGTACCCAGAACCAGAGCCACATTAACCGGCCCTGGGAATATAAGGTGGTC |
| | | CCAGCTCGGGGACACAGGATCCCTGGAGGCAGCAAACATGCTGTCCTGAAGTGGA |
| | | CATAGGGGCCCGGGTTGGAGGAAGAAGACTAGCTGAGCTCTCGGACCCCTGGAAG |
| | | ATGCCATGACAGGGGGCTGGAAGAGCTAGCACAGACTAGAGAGGTAAGGGGGGTA |
| | | GGGGAGCTGCCCAAATGAAAGGAGTGAGAGGTGACCCGAATCCACAGGAGAACGG |
| | | GGTGTCCAGGCAAAGAAAGCAAGAGGATGGAGAGGTGGCTAAAGCCAGGGAGACG |
| | | GGGTACTTTGGGGTTGTCCAGAAAAACGGTGATGATGCAGGCCTACAAGAAGGGG |
| | | AGGCGGGACGCAAGGGAGACATCCGTCGGAGAAGGCCATCCTAAGAAACGAGAGA |
| | | TGGCACAGGCCCCAGAAGGAGAAGGAAAAGGGAACCCAGCGAGTGAAGACGGCAT |
| | | GGGGTTGGGTGAGGGAGGAGAGATGCCCGGAGAGGACCCAGACACGGGGAGGATC |
| | | CGCTCAGAGGACATCACGTGGTGCAGCGCCGAGAAGGAAGTGCTCCGGAAAGAGC |
| | | ATCCTTGGGCAGCAACACAGCAGAGAGCAAGGGGAAGAGGGAGTGGAGGAAGACG |
| | | GAACCTGAAGGAGGCGGC |
| CTX945 | 83 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTTACGCG |
| | | TGCTAGCCCGGGCACTGACTCATCAAGCACTGACTCATCAAGCACTGACTCATCA |
| | | AGGGACTCAGGGAGGGAAACTCAGACGCTAGCGGGGGGCTATAAAAGGGGGTGGG |
| | | GGCGTTCGTCCTCACTCTAAGGCCAGCCCAGCACCAGCACCAGCCAACTCTCACT |
| | | GAAGCCAGCTCTCTCTTCCTCCACCACCATGGTGAGCAAGGGCGAGGAGCTGTTC |
| | | ACCGGGGTGGTGCCCATCCTGGTCGAGCTGGACGGCGACGTAAACGGCCACAAGT |
| | | TCAGCGTGTCCGGCGAGGGCGAGGGCGATGCCACCTACGGCAAGCTGACCCTGAA |
| | | GTTCATCTGCACCACCGGCAAGCTGCCCGTGCCCTGGCCCACCCTCGTGACCACC |
| | | CTGACCTACGGCGTGCAGTGCTTCAGCCGCTACCCCGACCACATGAAGCAGCACG |
| | | ACTTCTTCAAGTCCGCCATGCCCGAAGGCTACGTCCAGGAGCGCACCATCTTCTT |
| | | CAAGGACGACGGCAACTACAAGACCCGCGCCGAGGTGAAGTTCGAGGGCGACACC |
| | | CTGGTGAACCGCATCGAGCTGAAGGGCATCGACTTCAAGGAGGACGGCAACATCC |
| | | TGGGGCACAAGCTGGAGTACAACTACAACAGCCACAACGTCTATATCATGGCCGA |
| | | CAAGCAGAAGAACGGCATCAAGGTGAACTTCAAGATCCGCCACAACATCGAGGAC |
| | | GGCAGCGTGCAGCTCGCCGACCACTACCAGCAGAACACCCCCATCGGCGACGGCC |
| | | CCGTGCTGCTGCCCGACAACCACTACCTGAGCACCCAGTCCGCCCTGAGCAAAGA |
| | | CCCCAACGAGAAGCGCGATCACATGGTCCTGCTGGAGTTCGTGACCGCCGCCGGG |
| | | ATCACTCTCGGCATGGACGAGCTGTACAAGTCCGGACTCAGATCTCTCATGAAGC |
| | | AGATCCAGAGCCATGGCTTCCCGCCGGAGGTGGAGGAGCAGGATGATGGCACGCT |
| | | GCCCATGTCTTGTGCCCAGGAGAGCGGGATGGACCGTCACCCTGCAGCCTGTGCT |
| | | TCTGCTAGGATCAATGTGTAGAATAAAATCGCTATCCATCGAAGATGGATGTGTG |
| | | TTGGTTTTTTGTGTGACTGTGGGGTGGAGGGGACAGATAAAAGTACCCAGAACCA |
| | | GAGCCACATTAACCGGCCCTGGGAATATAAGGTGGTCCCAGCTCGGGGACACAGG |
| | | ATCCCTGGAGGCAGCAAACATGCTGTCCTGAAGTGGACATAGGGGCCCGGGTTGG |
| | | AGGAAGAAGACTAGCTGAGCTCTCGGACCCCTGGAAGATGCCATGACAGGGGGCT |
| | | GGAAGAGCTAGCACAGACTAGAGAGGTAAGGGGGGTAGGGGAGCTGCCCAAATGA |
| | | AAGGAGTGAGAGGTGACCCGAATCCACAGGAGAACGGGGTGTCCAGGCAAAGAAA |
| | | GCAAGAGGATGGAGAGGTGGCTAAAGCCAGGGAGACGGGGTACTTTGGGGTTGTC |
| | | CAGAAAAACGGTGATGATGCAGGCCTACAAGAAGGGGAGGCGGGACGCAAGGGAG |
| | | ACATCCGTCGGAGAAGGCCATCCTAAGAAACGAGAGATGGCACAGGCCCCAGAAG |
| | | GAGAAGGAAAAGGGAACCCAGCGAGTGAAGACGGCATGGGGTTGGGTGAGGGAGG |
| | | AGAGATGCCCGGAGAGGACCCAGACACGGGGAGGATCCGCTCAGAGGACATCACG |
| | | TGGTGCAGCGCCGAGAAGGAAGTGCTCCGGAAAGAGCATCCTTGGGCAGCAACAC |
| | | AGCAGAGAGCAAGGGGAAGAGGGAGTGGAGGAAGACGGAACCTGAAGGAGGCGGC |
| CTX946 | 84 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTGCCTAA |
| | | CTGGCCGGTACCTGAGCTCAGTTCTGAGAAAAGTAGTTCTGAGAAAAGTAGTTCT |
| | | GAGAAAAGTAGTTCTGAGAAAAGTAGTTCTGAGAAAAGTCAGACGCTAGCGGGGG |
| | | GCTATAAAAGGGGGTGGGGGCGTTCGTCCTCACTCTAAGGCCAGCCCAGCACCAG |
| | | CACCAGCCAACTCTCACTGAAGCCAGCTCTCTCTTCCTCCACCACCATGGTGAGC |
| | | AAGGGCGAGGAGCTGTTCACCGGGGTGGTGCCCATCCTGGTCGAGCTGGACGGCG |
| | | ACGTAAACGGCCACAAGTTCAGCGTGTCCGGCGAGGGCGAGGGCGATGCCACCTA |
| | | CGGCAAGCTGACCCTGAAGTTCATCTGCACCACCGGCAAGCTGCCCGTGCCCTGG |
| | | CCCACCCTCGTGACCACCCTGACCTACGGCGTGCAGTGCTTCAGCCGCTACCCCG |
| | | ACCACATGAAGCAGCACGACTTCTTCAAGTCCGCCATGCCCGAAGGCTACGTCCA |
| | | GGAGCGCACCATCTTCTTCAAGGACGACGGCAACTACAAGACCCGCGCCGAGGTG |
| | | AAGTTCGAGGGCGACACCCTGGTGAACCGCATCGAGCTGAAGGGCATCGACTTCA |
| | | AGGAGGACGGCAACATCCTGGGGCACAAGCTGGAGTACAACTACAACAGCCACAA |
| | | CGTCTATATCATGGCCGACAAGCAGAAGAACGGCATCAAGGTGAACTTCAAGATC |
| | | CGCCACAACATCGAGGACGGCAGCGTGCAGCTCGCCGACCACTACCAGCAGAACA |
| | | CCCCCATCGGCGACGGCCCCGTGCTGCTGCCCGACAACCACTACCTGAGCACCCA |
| | | GTCCGCCCTGAGCAAAGACCCCAACGAGAAGCGCGATCACATGGTCCTGCTGGAG |
| | | TTCGTGACCGCCGCCGGGATCACTCTCGGCATGGACGAGCTGTACAAGTCCGGAC |
| | | TCAGATCTCTCATGAAGCAGATCCAGAGCCATGGCTTCCCGCCGGAGGTGGAGGA |
| | | GCAGGATGATGGCACGCTGCCCATGTCTTGTGCCCAGGAGAGCGGGATGGACCGT |
| | | CACCCTGCAGCCTGTGCTTCTGCTAGGATCAATGTGTAGAATAAAATCGCTATCC |
| | | ATCGAAGATGGATGTGTGTTGGTTTTTTGTGTGACTGTGGGGTGGAGGGGACAGA |
| | | TAAAAGTACCCAGAACCAGAGCCACATTAACCGGCCCTGGGAATATAAGGTGGTC |
| | | CCAGCTCGGGGACACAGGATCCCTGGAGGCAGCAAACATGCTGTCCTGAAGTGGA |
| | | CATAGGGGCCCGGGTTGGAGGAAGAAGACTAGCTGAGCTCTCGGACCCCTGGAAG |
| | | ATGCCATGACAGGGGGCTGGAAGAGCTAGCACAGACTAGAGAGGTAAGGGGGGTA |
| | | GGGGAGCTGCCCAAATGAAAGGAGTGAGAGGTGACCCGAATCCACAGGAGAACGG |
| | | GGTGTCCAGGCAAAGAAAGCAAGAGGATGGAGAGGTGGCTAAAGCCAGGGAGACG |
| | | GGGTACTTTGGGGTTGTCCAGAAAAACGGTGATGATGCAGGCCTACAAGAAGGGG |
| | | AGGCGGGACGCAAGGGAGACATCCGTCGGAGAAGGCCATCCTAAGAAACGAGAGA |
| | | TGGCACAGGCCCCAGAAGGAGAAGGAAAAGGGAACCCAGCGAGTGAAGACGGCAT |
| | | GGGGTTGGGTGAGGGAGGAGAGATGCCCGGAGAGGACCCAGACACGGGGAGGATC |
| | | CGCTCAGAGGACATCACGTGGTGCAGCGCCGAGAAGGAAGTGCTCCGGAAAGAGC |
| | | ATCCTTGGGCAGCAACACAGCAGAGAGCAAGGGGAAGAGGGAGTGGAGGAAGACG |
| | | GAACCTGAAGGAGGCGGC |
| CTX947 | 85 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTATCGAT |
| | | AGGTACTAAGTCTAGACGGCAGTCTAGACGTACTAAGTCTAGACGGCAGTCTAGA |
| | | CGTACCGAGCTCTTACGCGTGCTAGCCCGGGCTCGAGATCTGCGATCAGACGCTA |
| | | GCGGGGGGCTATAAAAGGGGGTGGGGGCGTTCGTCCTCACTCTAAGGCCAGCCCA |
| | | GCACCAGCACCAGCCAACTCTCACTGAAGCCAGCTCTCTCTTCCTCCACCACCAT |
| | | GGTGAGCAAGGGCGAGGAGCTGTTCACCGGGGTGGTGCCCATCCTGGTCGAGCTG |
| | | GACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGT |
| | | GCCCTGGCCCACCCTCGTGACCACCCTGACCTACGGCGTGCAGTGCTTCAGCCGC |
| | | TACCCCGACCACATGAAGCAGCACGACTTCTTCAAGTCCGCCATGCCCGAAGGCT |
| | | ACGTCCAGGAGCGCACCATCTTCTTCAAGGACGACGGCAACTACAAGACCCGCGC |
| | | CGAGGTGAAGTTCGAGGGCGACACCCTGGTGAACCGCATCGAGCTGAAGGGCATC |
| | | GACTTCAAGGAGGACGGCAACATCCTGGGGCACAAGCTGGAGTACAACTACAACA |
| | | GCCACAACGTCTATATCATGGCCGACAAGCAGAAGAACGGCATCAAGGTGAACTT |
| | | CAAGATCCGCCACAACATCGAGGACGGCAGCGTGCAGCTCGCCGACCACTACCAG |
| | | CAGAACACCCCCATCGGCGACGGCCCCGTGCTGCTGCCCGACAACCACTACCTGA |
| | | GCACCCAGTCCGCCCTGAGCAAAGACCCCAACGAGAAGCGCGATCACATGGTCCT |
| | | GCTGGAGTTCGTGACCGCCGCCGGGATCACTCTCGGCATGGACGAGCTGTACAAG |
| | | TCCGGACTCAGATCTCTCATGAAGCAGATCCAGAGCCATGGCTTCCCGCCGGAGG |
| | | TGGAGGAGCAGGATGATGGCACGCTGCCCATGTCTTGTGCCCAGGAGAGCGGGAT |
| | | GGACCGTCACCCTGCAGCCTGTGCTTCTGCTAGGATCAATGTGTAGAATAAAATC |
| | | GCTATCCATCGAAGATGGATGTGTGTTGGTTTTTTGTGTGACTGTGGGGTGGAGG |
| | | GGACAGATAAAAGTACCCAGAACCAGAGCCACATTAACCGGCCCTGGGAATATAA |
| | | GGTGGTCCCAGCTCGGGGACACAGGATCCCTGGAGGCAGCAAACATGCTGTCCTG |
| | | AAGTGGACATAGGGGCCCGGGTTGGAGGAAGAAGACTAGCTGAGCTCTCGGACCC |
| | | CTGGAAGATGCCATGACAGGGGGCTGGAAGAGCTAGCACAGACTAGAGAGGTAAG |
| | | GGGGGTAGGGGAGCTGCCCAAATGAAAGGAGTGAGAGGTGACCCGAATCCACAGG |
| | | AGAACGGGGTGTCCAGGCAAAGAAAGCAAGAGGATGGAGAGGTGGCTAAAGCCAG |
| | | GGAGACGGGGTACTTTGGGGTTGTCCAGAAAAACGGTGATGATGCAGGCCTACAA |
| | | GAAGGGGAGGCGGGACGCAAGGGAGACATCCGTCGGAGAAGGCCATCCTAAGAAA |
| | | CGAGAGATGGCACAGGCCCCAGAAGGAGAAGGAAAAGGGAACCCAGCGAGTGAAG |
| | | ACGGCATGGGGTTGGGTGAGGGAGGAGAGATGCCCGGAGAGGACCCAGACACGGG |
| | | GAGGATCCGCTCAGAGGACATCACGTGGTGCAGCGCCGAGAAGGAAGTGCTCCGG |
| | | AAAGAGCATCCTTGGGCAGCAACACAGCAGAGAGCAAGGGGAAGAGGGAGTGGAG |
| | | GAAGACGGAACCTGAAGGAGGCGGC |
| B2M RHA | 86 | CCAGCGTGAGTCTCTCCTACCCTCCCGCTCTGGTCCTTCCTCTCCCGCTCTGCAC |
| | | CCTCTGTGGCCCTCGCTGTGCTCTCTCGCTCCGTGACTTCCCTTCTCCAAGTTCT |
| | | CCTTGGTGGCCCGCCGTGGGGCTAGTCCAGGGCTGGATCTCGGGGAAGCGGCGGG |
| | | GTGGCCTGGGAGTGGGGAAGGGGGTGCGCACCCGGGACGCGCGCTACTTGCCCCT |
| | | TTCGGCGGGGAGCAGGGGAGACCTTTGGCCTACGGCGACGGGAGGGTCGGGACAA |
| | | AGTTTAGGGCGTCGATAAGCGTCAGAGCGCCGAGGTTGGGGGAGGGTTTCTCTTC |
| | | CGCTCTTTCGCGGGGCCTCTGGCTCCCCCAGCGCAGCTGGAGTGGGGGACGGGTA |
| | | GGCTCGTCCCAAAGGCGCGGCGCTGAGGTTTGTGAACGCGTGGAGGGGCGCTTGG |
| | | GGTCTGGGGGAGGCGTCGCCCGGGTAAGCCTGTCTGCTGCGGCTCTGCTTCCCTT |
| | | AGACTGGAGAGCTGTGGACTTCGTCTAGGCGCCCGCTAAGTTCGCATGTCCTAGC |
| | | ACCTCTGGGTCTATGTGGGGCCACACCGTGGGGAGGAAACAGCACGCGACGTTTG |
| | | TAGAATGCTTGGCTGTGATACAAAGCGGTTTCGAATAATTAACTTATTTGTTCCC |
| | | ATCACATGTCACTTTTAAAAAATTATAAGAACTACCCGTTATTGACATCTTTCTG |
| | | TGTGCCAAGGACTTTATGTGCTTTGCGTCATTTAATTTTGAAAACAGTTATCTTC |
| | | CGCCATAGATAACTACTATGGTTATCTTCT |
| B2M LHA | 87 | GTTCTAGGGTGGAAACTAAGAGAATGATGTACCTAGAGGGCGCTGGAAGCTCTAA |
| | | AGCCCTAGCAGTTACTGCTTTTACTATTAGTGGTCGTTTTTTTCTCCCCCCCGCC |
| | | CCCCGACAAATCAACAGAACAAAGAAAATTACCTAAACAGCAAGGACATAGGGAG |
| | | GAACTTCTTGGCACAGAACTTTCCAAACACTTTTTCCTGAAGGGATACAAGAAGC |
| | | AAGAAAGGTACTCTTTCACTAGGACCTTCTCTGAGCTGTCCTCAGGATGCTTTTG |
| | | GGACTATTTTTCTTACCCAGAGAATGGAGAAACCCTGCAGGGAATTCCCAAGCTG |
| | | TAGTTATAAACAGAAGTTCTCCTTCTGCTAGGTAGCATTCAAAGATCTTAATCTT |
| | | CTGGGTTTCCGTTTTCTCGAATGAAAAATGCAGGTCCGAGCAGTTAACTGGCTGG |
| | | GGCACCATTAGCAAGTCACTTAGCATCTCTGGGGCCAGTCTGCAAAGCGAGGGGG |
| | | CAGCCTTAATGTGCCTCCAGCCTGAAGTCCTAGAATGAGCGCCCGGTGTCCCAAG |
| | | CTGGGGCGCGCACCCCAGATCGGAGGGCGCCGATGTACAGACAGCAAACTCACCC |
| | | AGTCTAGTGCATGCCTTCTTAAACATCACGAGACTCTAAGAAAAGGAAACTGAAA |
| | | ACGGGAAAGTCCCTCTCTCTAACCTGGCACTGCGTCGCTGGCTTGGAGACAGGTG |
| | | ACGGTCCCTGCGGGCCTTGTCCTGATTGGCTGGGCACGCGTTTAATATAAGTGGA |
| | | GGCGTCGCGCTGGCGGGCATTCCTGAAGCT |
| CD70 RHA | 88 | GGGCTTGGTGATCTGCCTCGTGGTGTGCATCCAGCGCTTCGCACAGGCTCAGCAG |
| | | CAGCTGCCGCTCGAGTCACTTGGGGTGAGTTGAGATGGAAAAGTTGGGAAGAAAA |
| | | CATAGAGAGGCGCGTGACCGAAAAGACAGAATGAGATGGGTACAAAGAGGCCAGA |
| | | GAGGAAGATCTGGTAGGGCAGAGACAGAGACCAGAACAGGGAGGCGAGGCGGGGA |
| | | CCAGGCTGCCCGGTGTAGGGGCTACGAGACAGGCAGCCCTGCCAGGAGGTACAGG |
| | | GAGATCCCGGGATGGGAAAGGTAGGCACACATGGAAATGGAAGATGACTCGGCTC |
| | | TGGTGTTCCCCCGGCAGGCTGACTCAGAGGCTGCTGGGGGCTTCACAAGGCTGGG |
| | | CGTGGGGGCTTCCTGGGGCCTCCTAGGACGGGATGGCCCCAGCCACTCGCTCCGG |
| | | GTGGGGGAGGGGTCCCTTTGGGGACCGCGCCGGGCGCCTTTGCAGCGTAGAGAGT |
| | | CCGCTGCGCGCGGTGCTCTCGCGCCCAGTGACATCCAGGAAAACGATTCGGGAAA |
| | | CGAAGAAGTTCTTTTGAAGGTCTCGACTTCACGTTCCCCGCTGGTTCAGACCTGC |
| | | TTCCTCTTTAAGAAGTCTTAAGAGTAAAAAAAAATAAAATGAAATAAAATCACCA |
| | | GTGCGCGCCGTGGGATGAGAGGTGGAAAGGAGGATGGACAGAGAAAAGAGAGCTC |
| | | CTGGCACAGGGGACACATAGAACCTCTCTGCTTACGTCCGTGCCCTGTTTTCTGG |
| | | TCTTTTCTTCCAGTGGGACGTAGCTGAGCT |
| CD70 LHA | 89 | GTTCAAGTCCAGCCTGGCCAACATGGTGAAACCCCATCTCTACTAAAAATACAAA |
| | | AAATTAGCCAGGCATGGTGGCGCGCGCATGTTACTCCCAGCTACTCGCGAGGCTC |
| | | AGACAGGAGAATCGCTTGAACCCAGGAGATCGAGGTTGCGGCGAGCTGAGATGGC |
| | | GCCACTGCACTCCAGCCTGGGTGACAGAGGGAGACCTCCGTCTCAAAAACAAAAC |
| | | AAATCAAAAAAATGCAGGAGAGGGGTACACGAATATTTGGGGAGCACCCCCAATT |
| | | CTTGGATGTCTGCTGTATCCCCAGTGCACAGCACAATCTAATCCCTAATAAATGT |
| | | GCAGTGGAGGTTTGTTGAATAAATGAATGGGCCCCAGAAGAATGAGGTGGAGAGG |
| | | GGAATAGGAAGATTGAATGTCTCCTGCCTGAAGGTCGGGCGGGGAGGGGTTGGGG |
| | | GCAGGCAACTCTGAGGCTCACCCGGGGCCACTGCCTGCATCCTGGCAACTGCCTC |
| | | CACCCACTTTAGGATCTTCAGACTGGCAGCGGTTGGAGGGAATTTCCCCTCGCCA |
| | | ATTGCTCAAGTCCCTCCCCTCGACCGGCCGGACATCCCCAGAGAGGGGCAGGCTG |
| | | GTCCCCTGACAGGTTGAAGCAAGTAGACGCCCAGGAGCCCCGGGAGGGGGCTGCA |
| | | GTTTCCTTCCTTCCTTCTCGGCAGCGCTCCGCGCCCCCATCGCCCCTCCTGCGCT |
| | | AGCGGAGGTGATCGCCGCGGCGATGCCGGAGGAGGGTTCGGGCTGCTCGGTGCGG |
| | | CGCAGGCCCTATGGGTGCGTCCTGCGGGCT |
| CTX1560* | 90 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTCATTTT |
| | | GACACCCCCATAATATTTTTCCAGAATTAACAGTATAAATTGCATCTCTTGTTCA |
| | | AGAGTTCCCTATCACTCTCTTTAATCACTACTCACAGTAACCTCAACTCCTGCAA |
| | | GGCCAGCCCAGCACCAGCACCAGCCAACTCTCACTGAAGCCAGCTCTCTCTTCCT |
| | | CCACCACCATGTGTCATCAGCAACTGGTCATCTCATGGTTTTCCCTGGTGTTTTT |
| | | GGCGTCACCACTGGTGGCAATATGGGAGCTTAAGAAGGACGTCTACGTTGTCGAG |
| | | CTGGATTGGTACCCAGACGCTCCAGGAGAAATGGTCGTTCTGACGTGTGACACAC |
| | | CTGAGGAAGATGGTATTACCTGGACGCTTGATCAGTCATCCGAAGTTCTTGGGTC |
| | | CGGGAAGACCCTTACAATCCAGGTCAAAGAGTTCGGAGATGCTGGTCAGTATACT |
| | | TGCCACAAGGGCGGTGAGGTCCTTAGTCACAGTTTGCTTCTGCTCCACAAGAAGG |
| | | AGGACGGCATATGGAGCACAGATATATTGAAAGACCAAAAAGAACCCAAAAATAA |
| | | GACATTCCTTCGCTGCGAGGCCAAGAACTACAGCGGCCGGTTTACGTGCTGGTGG |
| | | CTCACAACCATATCCACAGATCTGACGTTCAGTGTTAAATCCTCAAGGGGTAGTA |
| | | GCGATCCGCAAGGGGTTACGTGCGGTGCTGCTACCCTTAGTGCTGAAAGGGTCAG |
| | | AGGGGACAACAAAGAGTACGAATATAGTGTCGAATGCCAGGAAGATAGTGCGTGT |
| | | CCGGCGGCAGAAGAGTCACTGCCAATTGAGGTGATGGTCGACGCTGTGCACAAAT |
| | | TGAAATACGAGAATTATACCTCAAGTTTCTTCATCAGAGATATTATAAAGCCTGA |
| | | CCCGCCCAAAAATTTGCAACTCAAACCACTGAAAAATAGCCGCCAGGTGGAAGTC |
| | | TCATGGGAATATCCTGATACCTGGTCCACACCCCACTCCTATTTCTCACTCACAT |
| | | TTTGCGTTCAGGTCCAGGGAAAGTCCAAGCGAGAAAAAAAAGATCGCGTTTTCAC |
| | | GGACAAAACCTCAGCCACAGTGATTTGCCGCAAGAATGCTTCCATATCCGTACGC |
| | | GCTCAAGACAGGTATTACTCATCTTCATGGTCTGAATGGGCCTCTGTACCCTGTT |
| | | CAGGAGGAGGTGGCAGTGGCGGGGGCGGATCAGGCGGTGGAGGTAGCAGAAATTT |
| | | GCCAGTGGCAACGCCAGATCCTGGTATGTTCCCGTGCCTCCACCACTCTCAGAAC |
| | | CTCTTGAGGGCTGTGTCCAACATGTTGCAAAAGGCGCGCCAAACGCTCGAGTTTT |
| | | ACCCATGTACATCAGAGGAAATTGACCACGAGGACATTACGAAGGATAAAACCAG |
| | | CACAGTAGAGGCATGTCTGCCATTGGAACTCACGAAAAACGAATCATGCCTTAAC |
| | | AGCCGAGAGACTTCTTTCATCACTAACGGATCTTGTCTTGCCTCAAGAAAGACTT |
| | | CATTCATGATGGCCCTCTGCCTCTCCTCAATCTACGAAGACCTCAAAATGTACCA |
| | | AGTTGAGTTCAAGACCATGAACGCTAAACTCCTTATGGATCCAAAGCGCCAAATC |
| | | TTTTTGGACCAAAACATGTTGGCTGTGATAGACGAGCTGATGCAGGCTCTCAACT |
| | | TCAATAGCGAGACCGTGCCCCAAAAGTCATCCCTTGAAGAACCAGATTTTTATAA |
| | | AACGAAGATTAAATTGTGTATTCTGCTTCACGCTTTCCGGATCCGCGCTGTGACC |
| | | ATTGATCGAGTTATGTCTTATCTGAACGCCTCTTAATAATAAAATCGCTATCCAT |
| | | CGAAGATGGATGTGTGTTGGTTTTTTGTGTGACTGTGGGGTGGAGGGGACAGATA |
| | | AAAGTACCCAGAACCAGAGCCACATTAACCGGCCCTGGGAATATAAGGTGGTCCC |
| | | AGCTCGGGGACACAGGATCCCTGGAGGCAGCAAACATGCTGTCCTGAAGTGGACA |
| | | TAGGGGCCCGGGTTGGAGGAAGAAGACTAGCTGAGCTCTCGGACCCCTGGAAGAT |
| | | GCCATGACAGGGGGCTGGAAGAGCTAGCACAGACTAGAGAGGTAAGGGGGGTAGG |
| | | GGAGCTGCCCAAATGAAAGGAGTGAGAGGTGACCCGAATCCACAGGAGAACGGGG |
| | | TGTCCAGGCAAAGAAAGCAAGAGGATGGAGAGGTGGCTAAAGCCAGGGAGACGGG |
| | | GTACTTTGGGGTTGTCCAGAAAAACGGTGATGATGCAGGCCTACAAGAAGGGGAG |
| | | GCGGGACGCAAGGGAGACATCCGTCGGAGAAGGCCATCCTAAGAAACGAGAGATG |
| | | GCACAGGCCCCAGAAGGAGAAGGAAAAGGGAACCCAGCGAGTGAAGACGGCATGG |
| | | GGTTGGGTGAGGGAGGAGAGATGCCCGGAGAGGACCCAGACACGGGGAGGATCCG |
| | | CTCAGAGGACATCACGTGGTGCAGCGCCGAGAAGGAAGTGCTCCGGAAAGAGCAT |
| | | CCTTGGGCAGCAACACAGCAGAGAGCAAGGGGAAGAGGGAGTGGAGGAAGACGGA |
| | | ACCTGAAGGAGGCGGC |
| CTX1561 | 91 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTTACGCG |
| | | TGCTAGCCCGGGCACTGACTCATCAAGCACTGACTCATCAAGCACTGACTCATCA |
| | | AGGGACTCAGGGAGGGAAACTCCATTTTGACACCCCCATAATATTTTTCCAGAAT |
| | | TAACAGTATAAATTGCATCTCTTGTTCAAGAGTTCCCTATCACTCTCTTTAATCA |
| | | CTACTCACAGTAACCTCAACTCCTGCAAGGCCAGCCCAGCACCAGCACCAGCCAA |
| | | CTCTCACTGAAGCCAGCTCTCTCTTCCTCCACCACCATGTGTCATCAGCAACTGG |
| | | TCATCTCATGGTTTTCCCTGGTGTTTTTGGCGTCACCACTGGTGGCAATATGGGA |
| | | GCTTAAGAAGGACGTCTACGTTGTCGAGCTGGATTGGTACCCAGACGCTCCAGGA |
| | | GAAATGGTCGTTCTGACGTGTGACACACCTGAGGAAGATGGTATTACCTGGACGC |
| | | TTGATCAGTCATCCGAAGTTCTTGGGTCCGGGAAGACCCTTACAATCCAGGTCAA |
| | | AGAGTTCGGAGATGCTGGTCAGTATACTTGCCACAAGGGCGGTGAGGTCCTTAGT |
| | | CACAGTTTGCTTCTGCTCCACAAGAAGGAGGACGGCATATGGAGCACAGATATAT |
| | | TGAAAGACCAAAAAGAACCCAAAAATAAGACATTCCTTCGCTGCGAGGCCAAGAA |
| | | CTACAGCGGCCGGTTTACGTGCTGGTGGCTCACAACCATATCCACAGATCTGACG |
| | | TTCAGTGTTAAATCCTCAAGGGGTAGTAGCGATCCGCAAGGGGTTACGTGCGGTG |
| | | CTGCTACCCTTAGTGCTGAAAGGGTCAGAGGGGACAACAAAGAGTACGAATATAG |
| | | TGTCGAATGCCAGGAAGATAGTGCGTGTCCGGCGGCAGAAGAGTCACTGCCAATT |
| | | GAGGTGATGGTCGACGCTGTGCACAAATTGAAATACGAGAATTATACCTCAAGTT |
| | | TCTTCATCAGAGATATTATAAAGCCTGACCCGCCCAAAAATTTGCAACTCAAACC |
| | | ACTGAAAAATAGCCGCCAGGTGGAAGTCTCATGGGAATATCCTGATACCTGGTCC |
| | | ACACCCCACTCCTATTTCTCACTCACATTTTGCGTTCAGGTCCAGGGAAAGTCCA |
| | | AGCGAGAAAAAAAAGATCGCGTTTTCACGGACAAAACCTCAGCCACAGTGATTTG |
| | | CCGCAAGAATGCTTCCATATCCGTACGCGCTCAAGACAGGTATTACTCATCTTCA |
| | | TGGTCTGAATGGGCCTCTGTACCCTGTTCAGGAGGAGGTGGCAGTGGCGGGGGCG |
| | | GATCAGGCGGTGGAGGTAGCAGAAATTTGCCAGTGGCAACGCCAGATCCTGGTAT |
| | | GTTCCCGTGCCTCCACCACTCTCAGAACCTCTTGAGGGCTGTGTCCAACATGTTG |
| | | CAAAAGGCGCGCCAAACGCTCGAGTTTTACCCATGTACATCAGAGGAAATTGACC |
| | | ACGAGGACATTACGAAGGATAAAACCAGCACAGTAGAGGCATGTCTGCCATTGGA |
| | | ACTCACGAAAAACGAATCATGCCTTAACAGCCGAGAGACTTCTTTCATCACTAAC |
| | | GGATCTTGTCTTGCCTCAAGAAAGACTTCATTCATGATGGCCCTCTGCCTCTCCT |
| | | CAATCTACGAAGACCTCAAAATGTACCAAGTTGAGTTCAAGACCATGAACGCTAA |
| | | ACTCCTTATGGATCCAAAGCGCCAAATCTTTTTGGACCAAAACATGTTGGCTGTG |
| | | ATAGACGAGCTGATGCAGGCTCTCAACTTCAATAGCGAGACCGTGCCCCAAAAGT |
| | | CATCCCTTGAAGAACCAGATTTTTATAAAACGAAGATTAAATTGTGTATTCTGCT |
| | | TCACGCTTTCCGGATCCGCGCTGTGACCATTGATCGAGTTATGTCTTATCTGAAC |
| | | GCCTCTTAAAATAAAATCGCTATCCATCGAAGATGGATGTGTGTTGGTTTTTTGT |
| | | GTGACTGTGGGGTGGAGGGGACAGATAAAAGTACCCAGAACCAGAGCCACATTAA |
| | | CCGGCCCTGGGAATATAAGGTGGTCCCAGCTCGGGGACACAGGATCCCTGGAGGC |
| | | AGCAAACATGCTGTCCTGAAGTGGACATAGGGGCCCGGGTTGGAGGAAGAAGACT |
| | | AGCTGAGCTCTCGGACCCCTGGAAGATGCCATGACAGGGGGCTGGAAGAGCTAGC |
| | | ACAGACTAGAGAGGTAAGGGGGGTAGGGGAGCTGCCCAAATGAAAGGAGTGAGAG |
| | | GTGACCCGAATCCACAGGAGAACGGGGTGTCCAGGCAAAGAAAGCAAGAGGATGG |
| | | AGAGGTGGCTAAAGCCAGGGAGACGGGGTACTTTGGGGTTGTCCAGAAAAACGGT |
| | | GATGATGCAGGCCTACAAGAAGGGGAGGCGGGACGCAAGGGAGACATCCGTCGGA |
| | | GAAGGCCATCCTAAGAAACGAGAGATGGCACAGGCCCCAGAAGGAGAAGGAAAAG |
| | | GGAACCCAGCGAGTGAAGACGGCATGGGGTTGGGTGAGGGAGGAGAGATGCCCGG |
| | | AGAGGACCCAGACACGGGGAGGATCCGCTCAGAGGACATCACGTGGTGCAGCGCC |
| | | GAGAAGGAAGTGCTCCGGAAAGAGCATCCTTGGGCAGCAACACAGCAGAGAGCAA |
| | | GGGGAAGAGGGAGTGGAGGAAGACGGAACCTGAAGGAGGCGGC |
| CTX1562* | 92 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTCATTTT |
| | | GACACCCCCATAATATTTTTCCAGAATTAACAGTATAAATTGCATCTCTTGTTCA |
| | | AGAGTTCCCTATCACTCTCTTTAATCACTACTCACAGTAACCTCAACTCCTGCAA |
| | | GGCCAGCCCAGCACCAGCACCAGCCAACTCTCACTGAAGCCAGCTCTCTCTTCCT |
| | | CCACCACCATGTGCCCAGCCCGGAGTCTTCTGCTGGTAGCAACATTGGTTCTCCT |
| | | GGACCATTTGTCACTGGCAAGAAACCTGCCGGTAGCAACCCCCGATCCTGGTATG |
| | | TTCCCTTGTTTGCATCACTCACAAAACCTTCTCCGCGCCGTTTCTAATATGCTGC |
| | | AAAAGGCACGGCAGACCCTTGAAXTTTACCCGTGTACATCCGAAGAAATCGACCA |
| | | TGAAGACATTACCAAGGATAAGACCTCCACGGTGGAAGCTTGTCTCCCTTTGGAA |
| | | CTTACCAAGAATGAAAGCTGCCTTAACTCTCGAGAGACTTCTTTCATCACTAATG |
| | | GAAGCTGCCTGGCGTCCCGGAAAACGTCCTTCATGATGGCGCTTTGTCTCTCCTC |
| | | CATCTACGAGGATCTCAAAATGTACCAGGTGGAATTTAAGACGATGAACGCAAAG |
| | | CTTCTGATGGATCCCAAGAGACAGATATTTCTGGACCAAAACATGTTGGCTGTCA |
| | | TCGACGAACTCATGCAGGCTTTGAATTTTAACTCCGAGACGGTGCCACAGAAGTC |
| | | CTCCCTCGAAGAACCGGATTTCTATAAGACTAAAATTAAATTGTGCATCCTGTTG |
| | | CACGCGTTTCGCATTCGGGCCGTCACAATTGACAGAGTAATGAGTTACCTGAACG |
| | | CCTCAGGTGGGGGTGGCTCCGGTGGAGGAGGATCAGGCGGTGGTGGCAGTATTTG |
| | | GGAATTGAAAAAGGATGTCTATGTTGTAGAACTTGATTGGTATCCGGACGCTCCA |
| | | GGTGAAATGGTCGTTCTGACGTGCGATACACCTGAGGAAGATGGGATCACATGGA |
| | | CACTCGACCAGAGCTCTGAGGTCCTCGGTAGCGGCAAGACGCTCACAATCCAGGT |
| | | TAAGGAGTTCGGGGACGCGGGGCAGTATACTTGCCATAAGGGCGGGGAAGTGCTC |
| | | TCTCATAGCCTGCTCCTTCTGCACAAGAAGGAAGATGGGATATGGTCCACGGACA |
| | | TCCTTAAAGACCAAAAGGAGCCAAAGAATAAAACGTTTCTCAGGTGTGAAGCGAA |
| | | AAACTATTCTGGGAGGTTTACCTGTTGGTGGCTCACGACGATCTCCACAGACTTG |
| | | ACATTCAGTGTTAAATCTAGCAGGGGATCATCTGACCCACAGGGAGTAACTTGTG |
| | | GGGCCGCAACTCTCTCAGCCGAGAGAGTGAGAGGGGACAATAAAGAGTACGAATA |
| | | TTCAGTAGAGTGCCAAGAGGACAGCGCCTGCCCCGCTGCGGAAGAAAGTCTGCCG |
| | | ATTGAAGTCATGGTCGACGCCGTCCATAAGTTGAAGTACGAAAATTACACGTCTT |
| | | CTTTTTTTATTCGAGACATAATAAAACCAGACCCCCCAAAAAATCTCCAACTGAA |
| | | GCCCTTGAAAAACTCACGCCAGGTTGAAGTGAGCTGGGAATATCCCGACACCTGG |
| | | TCCACGCCGCATTCTTATTTTAGCTTGACGTTTTGTGTACAGGTTCAGGGTAAGA |
| | | GTAAACGAGAAAAAAAAGACCGAGTTTTTACAGACAAGACTTCTGCCACAGTCAT |
| | | CTGCAGAAAAAATGCAAGTATCAGTGTAAGAGCGCAGGACCGCTACTACTCTTCC |
| | | TCTTGGAGCGAGTGGGCGTCAGTTCCTTGCAGCTAATAATAAAATCGCTATCCAT |
| | | CGAAGATGGATGTGTGTTGGTTTTTTGTGTGACTGTGGGGTGGAGGGGACAGATA |
| | | AAAGTACCCAGAACCAGAGCCACATTAACCGGCCCTGGGAATATAAGGTGGTCCC |
| | | AGCTCGGGGACACAGGATCCCTGGAGGCAGCAAACATGCTGTCCTGAAGTGGACA |
| | | TAGGGGCCCGGGTTGGAGGAAGAAGACTAGCTGAGCTCTCGGACCCCTGGAAGAT |
| | | GCCATGACAGGGGGCTGGAAGAGCTAGCACAGACTAGAGAGGTAAGGGGGGTAGG |
| | | GGAGCTGCCCAAATGAAAGGAGTGAGAGGTGACCCGAATCCACAGGAGAACGGGG |
| | | TGTCCAGGCAAAGAAAGCAAGAGGATGGAGAGGTGGCTAAAGCCAGGGAGACGGG |
| | | GTACTTTGGGGTTGTCCAGAAAAACGGTGATGATGCAGGCCTACAAGAAGGGGAG |
| | | GCGGGACGCAAGGGAGACATCCGTCGGAGAAGGCCATCCTAAGAAACGAGAGATG |
| | | GCACAGGCCCCAGAAGGAGAAGGAAAAGGGAACCCAGCGAGTGAAGACGGCATGG |
| | | GGTTGGGTGAGGGAGGAGAGATGCCCGGAGAGGACCCAGACACGGGGAGGATCCG |
| | | CTCAGAGGACATCACGTGGTGCAGCGCCGAGAAGGAAGTGCTCCGGAAAGAGCAT |
| | | CCTTGGGCAGCAACACAGCAGAGAGCAAGGGGAAGAGGGAGTGGAGGAAGACGGA |
| | | ACCTGAAGGAGGCGGC |
| CTX1563 | 93 | GAAGCCCAGAGCAGGGCCTTAGGGAAGCGGGACCCTGCTCTGGGCGGAGGAATAT |
| | | GTCCCAGATAGCACTGGGGACTCTTTAAGGAAAGAAGGATGGAGAAAGAGAAAGG |
| | | GAGTAGAGGCGGCCACGACCTGGTGAACACCTAGGACGCACCATTCTCACAAAGG |
| | | GAGTTTTCCACACGGACACCCCCCTCCTCACCACAGCCCTGCCAGGACGGGGCTG |
| | | GCTACTGGCCTTATCTCACAGGTAAAACTGACGCACGGAGGAACAATATAAATTG |
| | | GGGACTAGAAAGGTGAAGAGCCAAAGTTAGAACTCAGGACCAACTTATTCTGATT |
| | | TTGTTTTTCCAAACTGCTTCTCCTCTTGGGAAGTGTAAGGAAGCTGCAGCACCAG |
| | | GATCAGTGAAACGCACCAGACGGCCGCGTCAGAGCAGCTCAGGTTCTGGGAGAGG |
| | | GTAGCGCAGGGTGGCCACTGAGAACCGGGCAGGTCACGCATCCCCCCCTTCCCTC |
| | | CCACCCCCTGCCAAGCTCTCCCTCCCAGGATCCTCTCTGGCTCCATCGTAAGCAA |
| | | ACCTTAGAGGTTCTGGCAAGGAGAGAGATGGCTCCAGGAAATGGGGGTGTGTCAC |
| | | CAGATAAGGAATCTGCCTAACAGGAGGTGGGGGTTAGACCCAATATCAGGAGACT |
| | | AGGAAGGAGGAGGCCTAAGGATGGGGCTTTTCTGTCACCAGCCACTAGTTACGCG |
| | | TGCTAGCCCGGGCACTGACTCATCAAGCACTGACTCATCAAGCACTGACTCATCA |
| | | AGGGACTCAGGGAGGGAAACTCCATTTTGACACCCCCATAATATTTTTCCAGAAT |
| | | TAACAGTATAAATTGCATCTCTTGTTCAAGAGTTCCCTATCACTCTCTTTAATCA |
| | | CTACTCACAGTAACCTCAACTCCTGCAAGGCCAGCCCAGCACCAGCACCAGCCAA |
| | | CTCTCACTGAAGCCAGCTCTCTCTTCCTCCACCACCATGTGCCCAGCCCGGAGTC |
| | | TTCTGCTGGTAGCAACATTGGTTCTCCTGGACCATTTGTCACTGGCAAGAAACCT |
| | | GCCGGTAGCAACCCCCGATCCTGGTATGTTCCCTTGTTTGCATCACTCACAAAAC |
| | | CTTCTCCGCGCCGTTTCTAATATGCTGCAAAAGGCACGGCAGACCCTTGAATTTT |
| | | ACCCGTGTACATCCGAAGAAATCGACCATGAAGACATTACCAAGGATAAGACCTC |
| | | CACGGTGGAAGCTTGTCTCCCTTTGGAACTTACCAAGAATGAAAGCTGCCTTAAC |
| | | TCTCGAGAGACTTCTTTCATCACTAATGGAAGCTGCCTGGCGTCCCGGAAAACGT |
| | | CCTTCATGATGGCGCTTTGTCTCTCCTCCATCTACGAGGATCTCAAAATGTACCA |
| | | GGTGGAATTTAAGACGATGAACGCAAAGCTTCTGATGGATCCCAAGAGACAGATA |
| | | TTTCTGGACCAAAACATGTTGGCTGTCATCGACGAACTCATGCAGGCTTTGAATT |
| | | TTAACTCCGAGACGGTGCCACAGAAGTCCTCCCTCGAAGAACCGGATTTCTATAA |
| | | GACTAAAATTAAATTGTGCATCCTGTTGCACGCGTTTCGCATTCGGGCCGTCACA |
| | | ATTGACAGAGTAATGAGTTACCTGAACGCCTCAGGTGGGGGTGGCTCCGGTGGAG |
| | | GAGGATCAGGCGGTGGTGGCAGTATTTGGGAATTGAAAAAGGATGTCTATGTTGT |
| | | AGAACTTGATTGGTATCCGGACGCTCCAGGTGAAATGGTCGTTCTGACGTGCGAT |
| | | ACACCTGAGGAAGATGGGATCACATGGACACTCGACCAGAGCTCTGAGGTCCTCG |
| | | GTAGCGGCAAGACGCTCACAATCCAGGTTAAGGAGTTCGGGGACGCGGGGCAGTA |
| | | TACTTGCCATAAGGGCGGGGAAGTGCTCTCTCATAGCCTGCTCCTTCTGCACAAG |
| | | AAGGAAGATGGGATATGGTCCACGGACATCCTTAAAGACCAAAAGGAGCCAAAGA |
| | | ATAAAACGTTTCTCAGGTGTGAAGCGAAAAACTATTCTGGGAGGTTTACCTGTTG |
| | | GTGGCTCACGACGATCTCCACAGACTTGACATTCAGTGTTAAATCTAGCAGGGGA |
| | | TCATCTGACCCACAGGGAGTAACTTGTGGGGCCGCAACTCTCTCAGCCGAGAGAG |
| | | TGAGAGGGGACAATAAAGAGTACGAATATTCAGTAGAGTGCCAAGAGGACAGCGC |
| | | CTGCCCCGCTGCGGAAGAAAGTCTGCCGATTGAAGTCATGGTCGACGCCGTCCAT |
| | | AAGTTGAAGTACGAAAATTACACGTCTTCTTTTTTTATTCGAGACATAATAAAAC |
| | | CAGACCCCCCAAAAAATCTCCAACTGAAGCCCTTGAAAAACTCACGCCAGGTTGA |
| | | AGTGAGCTGGGAATATCCCGACACCTGGTCCACGCCGCATTCTTATTTTAGCTTG |
| | | ACGTTTTGTGTACAGGTTCAGGGTAAGAGTAAACGAGAAAAAAAAGACCGAGTTT |
| | | TTACAGACAAGACTTCTGCCACAGTCATCTGCAGAAAAAATGCAAGTATCAGTGT |
| | | AAGAGCGCAGGACCGCTACTACTCTTCCTCTTGGAGCGAGTGGGCGTCAGTTCCT |
| | | TGCAGCTAAAATAAAATCGCTATCCATCGAAGATGGATGTGTGTTGGTTTTTTGT |
| | | GTGACTGTGGGGTGGAGGGGACAGATAAAAGTACCCAGAACCAGAGCCACATTAA |
| | | CCGGCCCTGGGAATATAAGGTGGTCCCAGCTCGGGGACACAGGATCCCTGGAGGC |
| | | AGCAAACATGCTGTCCTGAAGTGGACATAGGGGCCCGGGTTGGAGGAAGAAGACT |
| | | AGCTGAGCTCTCGGACCCCTGGAAGATGCCATGACAGGGGGCTGGAAGAGCTAGC |
| | | ACAGACTAGAGAGGTAAGGGGGGTAGGGGAGCTGCCCAAATGAAAGGAGTGAGAG |
| | | GTGACCCGAATCCACAGGAGAACGGGGTGTCCAGGCAAAGAAAGCAAGAGGATGG |
| | | AGAGGTGGCTAAAGCCAGGGAGACGGGGTACTTTGGGGTTGTCCAGAAAAACGGT |
| | | GATGATGCAGGCCTACAAGAAGGGGAGGCGGGACGCAAGGGAGACATCCGTCGGA |
| | | GAAGGCCATCCTAAGAAACGAGAGATGGCACAGGCCCCAGAAGGAGAAGGAAAAG |
| | | GGAACCCAGCGAGTGAAGACGGCATGGGGTTGGGTGAGGGAGGAGAGATGCCCGG |
| | | AGAGGACCCAGACACGGGGAGGATCCGCTCAGAGGACATCACGTGGTGCAGCGCC |
| | | GAGAAGGAAGTGCTCCGGAAAGAGCATCCTTGGGCAGCAACACAGCAGAGAGCAA |
| | | GGGGAAGAGGGAGTGGAGGAAGACGGAACCTGAAGGAGGCGGC |
| CTX1564* | 94 | GTTCTAGGGTGGAAACTAAGAGAATGATGTACCTAGAGGGCGCTGGAAGCTCTAA |
| | | AGCCCTAGCAGTTACTGCTTTTACTATTAGTGGTCGTTTTTTTCTCCCCCCCGCC |
| | | CCCCGACAAATCAACAGAACAAAGAAAATTACCTAAACAGCAAGGACATAGGGAG |
| | | GAACTTCTTGGCACAGAACTTTCCAAACACTTTTTCCTGAAGGGATACAAGAAGC |
| | | AAGAAAGGTACTCTTTCACTAGGACCTTCTCTGAGCTGTCCTCAGGATGCTTTTG |
| | | GGACTATTTTTCTTACCCAGAGAATGGAGAAACCCTGCAGGGAATTCCCAAGCTG |
| | | TAGTTATAAACAGAAGTTCTCCTTCTGCTAGGTAGCATTCAAAGATCTTAATCTT |
| | | CTGGGTTTCCGTTTTCTCGAATGAAAAATGCAGGTCCGAGCAGTTAACTGGCTGG |
| | | GGCACCATTAGCAAGTCACTTAGCATCTCTGGGGCCAGTCTGCAAAGCGAGGGGG |
| | | CAGCCTTAATGTGCCTCCAGCCTGAAGTCCTAGAATGAGCGCCCGGTGTCCCAAG |
| | | CTGGGGCGCGCACCCCAGATCGGAGGGCGCCGATGTACAGACAGCAAACTCACCC |
| | | AGTCTAGTGCATGCCTTCTTAAACATCACGAGACTCTAAGAAAAGGAAACTGAAA |
| | | ACGGGAAAGTCCCTCTCTCTAACCTGGCACTGCGTCGCTGGCTTGGAGACAGGTG |
| | | ACGGTCCCTGCGGGCCTTGTCCTGATTGGCTGGGCACGCGTTTAATATAAGTGGA |
| | | GGCGTCGCGCTGGCGGGCATTCCTGAAGCTTCATTTTGACACCCCCATAATATTT |
| | | TTCCAGAATTAACAGTATAAATTGCATCTCTTGTTCAAGAGTTCCCTATCACTCT |
| | | CTTTAATCACTACTCACAGTAACCTCAACTCCTGCAAGGCCAGCCCAGCACCAGC |
| | | ACCAGCCAACTCTCACTGAAGCCAGCTCTCTCTTCCTCCACCACCATGTGTCATC |
| | | AGCAACTGGTCATCTCATGGTTTTCCCTGGTGTTTTTGGCGTCACCACTGGTGGC |
| | | AATATGGGAGCTTAAGAAGGACGTCTACGTTGTCGAGCTGGATTGGTACCCAGAC |
| | | GCTCCAGGAGAAATGGTCGTTCTGACGTGTGACACACCTGAGGAAGATGGTATTA |
| | | CCTGGACGCTTGATCAGTCATCCGAAGTTCTTGGGTCCGGGAAGACCCTTACAAT |
| | | CCAGGTCAAAGAGTTCGGAGATGCTGGTCAGTATACTTGCCACAAGGGCGGTGAG |
| | | GTCCTTAGTCACAGTTTGCTTCTGCTCCACAAGAAGGAGGACGGCATATGGAGCA |
| | | CAGATATATTGAAAGACCAAAAAGAACCCAAAAATAAGACATTCCTTCGCTGCGA |
| | | GGCCAAGAACTACAGCGGCCGGTTTACGTGCTGGTGGCTCACAACCATATCCACA |
| | | GATCTGACGTTCAGTGTTAAATCCTCAAGGGGTAGTAGCGATCCGCAAGGGGTTA |
| | | CGTGCGGTGCTGCTACCCTTAGTGCTGAAAGGGTCAGAGGGGACAACAAAGAGTA |
| | | CGAATATAGTGTCGAATGCCAGGAAGATAGTGCGTGTCCGGCGGCAGAAGAGTCA |
| | | CTGCCAATTGAGGTGATGGTCGACGCTGTGCACAAATTGAAATACGAGAATTATA |
| | | CCTCAAGTTTCTTCATCAGAGATATTATAAAGCCTGACCCGCCCAAAAATTTGCA |
| | | ACTCAAACCACTGAAAAATAGCCGCCAGGTGGAAGTCTCATGGGAATATCCTGAT |
| | | ACCTGGTCCACACCCCACTCCTATTTCTCACTCACATTTTGCGTTCAGGTCCAGG |
| | | GAAAGTCCAAGCGAGAAAAAAAAGATCGCGTTTTCACGGACAAAACCTCAGCCAC |
| | | AGTGATTTGCCGCAAGAATGCTTCCATATCCGTACGCGCTCAAGACAGGTATTAC |
| | | TCATCTTCATGGTCTGAATGGGCCTCTGTACCCTGTTCAGGAGGAGGTGGCAGTG |
| | | GCGGGGGCGGATCAGGCGGTGGAGGTAGCAGAAATTTGCCAGTGGCAACGCCAGA |
| | | TCCTGGTATGTTCCCGTGCCTCCACCACTCTCAGAACCTCTTGAGGGCTGTGTCC |
| | | AACATGTTGCAAAAGGCGCGCCAAACGCTCGAGTTTTACCCATGTACATCAGAGG |
| | | AAATTGACCACGAGGACATTACGAAGGATAAAACCAGCACAGTAGAGGCATGTCT |
| | | GCCATTGGAACTCACGAAAAACGAATCATGCCTTAACAGCCGAGAGACTTCTTTC |
| | | ATCACTAACGGATCTTGTCTTGCCTCAAGAAAGACTTCATTCATGATGGCCCTCT |
| | | GCCTCTCCTCAATCTACGAAGACCTCAAAATGTACCAAGTTGAGTTCAAGACCAT |
| | | GAACGCTAAACTCCTTATGGATCCAAAGCGCCAAATCTTTTTGGACCAAAACATG |
| | | TTGGCTGTGATAGACGAGCTGATGCAGGCTCTCAACTTCAATAGCGAGACCGTGC |
| | | CCCAAAAGTCATCCCTTGAAGAACCAGATTTTTATAAAACGAAGATTAAATTGTG |
| | | TATTCTGCTTCACGCTTTCCGGATCCGCGCTGTGACCATTGATCGAGTTATGTCT |
| | | TATCTGAACGCCTCTTAATAATAAAATCGCTATCCATCGAAGATGGATGTGTGTT |
| | | GGTTTTTTGTGTGCCAGCGTGAGTCTCTCCTACCCTCCCGCTCTGGTCCTTCCTC |
| | | TCCCGCTCTGCACCCTCTGTGGCCCTCGCTGTGCTCTCTCGCTCCGTGACTTCCC |
| | | TTCTCCAAGTTCTCCTTGGTGGCCCGCCGTGGGGCTAGTCCAGGGCTGGATCTCG |
| | | GGGAAGCGGCGGGGTGGCCTGGGAGTGGGGAAGGGGGTGCGCACCCGGGACGCGC |
| | | GCTACTTGCCCCTTTCGGCGGGGAGCAGGGGAGACCTTTGGCCTACGGCGACGGG |
| | | AGGGTCGGGACAAAGTTTAGGGCGTCGATAAGCGTCAGAGCGCCGAGGTTGGGGG |
| | | AGGGTTTCTCTTCCGCTCTTTCGCGGGGCCTCTGGCTCCCCCAGCGCAGCTGGAG |
| | | TGGGGGACGGGTAGGCTCGTCCCAAAGGCGCGGCGCTGAGGTTTGTGAACGCGTG |
| | | GAGGGGCGCTTGGGGTCTGGGGGAGGCGTCGCCCGGGTAAGCCTGTCTGCTGCGG |
| | | CTCTGCTTCCCTTAGACTGGAGAGCTGTGGACTTCGTCTAGGCGCCCGCTAAGTT |
| | | CGCATGTCCTAGCACCTCTGGGTCTATGTGGGGCCACACCGTGGGGAGGAAACAG |
| | | CACGCGACGTTTGTAGAATGCTTGGCTGTGATACAAAGCGGTTTCGAATAATTAA |
| | | CTTATTTGTTCCCATCACATGTCACTTTTAAAAAATTATAAGAACTACCCGTTAT |
| | | TGACATCTTTCTGTGTGCCAAGGACTTTATGTGCTTTGCGTCATTTAATTTTGAA |
| | | AACAGTTATCTTCCGCCATAGATAACTACTATGGTTATCTTC |
| CTX1565 | 95 | GTTCTAGGGTGGAAACTAAGAGAATGATGTACCTAGAGGGCGCTGGAAGCTCTAA |
| | | AGCCCTAGCAGTTACTGCTTTTACTATTAGTGGTCGTTTTTTTCTCCCCCCCGCC |
| | | CCCCGACAAATCAACAGAACAAAGAAAATTACCTAAACAGCAAGGACATAGGGAG |
| | | GAACTTCTTGGCACAGAACTTTCCAAACACTTTTTCCTGAAGGGATACAAGAAGC |
| | | AAGAAAGGTACTCTTTCACTAGGACCTTCTCTGAGCTGTCCTCAGGATGCTTTTG |
| | | GGACTATTTTTCTTACCCAGAGAATGGAGAAACCCTGCAGGGAATTCCCAAGCTG |
| | | TAGTTATAAACAGAAGTTCTCCTTCTGCTAGGTAGCATTCAAAGATCTTAATCTT |
| | | CTGGGTTTCCGTTTTCTCGAATGAAAAATGCAGGTCCGAGCAGTTAACTGGCTGG |
| | | GGCACCATTAGCAAGTCACTTAGCATCTCTGGGGCCAGTCTGCAAAGCGAGGGGG |
| | | CAGCCTTAATGTGCCTCCAGCCTGAAGTCCTAGAATGAGCGCCCGGTGTCCCAAG |
| | | CTGGGGCGCGCACCCCAGATCGGAGGGCGCCGATGTACAGACAGCAAACTCACCC |
| | | AGTCTAGTGCATGCCTTCTTAAACATCACGAGACTCTAAGAAAAGGAAACTGAAA |
| | | ACGGGAAAGTCCCTCTCTCTAACCTGGCACTGCGTCGCTGGCTTGGAGACAGGTG |
| | | ACGGTCCCTGCGGGCCTTGTCCTGATTGGCTGGGCACGCGTTTAATATAAGTGGA |
| | | GGCGTCGCGCTGGCGGGCATTCCTGAAGCTTACGCGTGCTAGCCCGGGCACTGAC |
| | | TCATCAAGCACTGACTCATCAAGCACTGACTCATCAAGGGACTCAGGGAGGGAAA |
| | | CTCCATTTTGACACCCCCATAATATTTTTCCAGAATTAACAGTATAAATTGCATC |
| | | TCTTGTTCAAGAGTTCCCTATCACTCTCTTTAATCACTACTCACAGTAACCTCAA |
| | | CTCCTGCAAGGCCAGCCCAGCACCAGCACCAGCCAACTCTCACTGAAGCCAGCTC |
| | | TCTCTTCCTCCACCACCATGTGTCATCAGCAACTGGTCATCTCATGGTTTTCCCT |
| | | GGTGTTTTTGGCGTCACCACTGGTGGCAATATGGGAGCTTAAGAAGGACGTCTAC |
| | | GTTGTCGAGCTGGATTGGTACCCAGACGCTCCAGGAGAAATGGTCGTTCTGACGT |
| | | GTGACACACCTGAGGAAGATGGTATTACCTGGACGCTTGATCAGTCATCCGAAGT |
| | | TCTTGGGTCCGGGAAGACCCTTACAATCCAGGTCAAAGAGTTCGGAGATGCTGGT |
| | | CAGTATACTTGCCACAAGGGCGGTGAGGTCCTTAGTCACAGTTTGCTTCTGCTCC |
| | | ACAAGAAGGAGGACGGCATATGGAGCACAGATATATTGAAAGACCAAAAAGAACC |
| | | CAAAAATAAGACATTCCTTCGCTGCGAGGCCAAGAACTACAGCGGCCGGTTTACG |
| | | TGCTGGTGGCTCACAACCATATCCACAGATCTGACGTTCAGTGTTAAATCCTCAA |
| | | GGGGTAGTAGCGATCCGCAAGGGGTTACGTGCGGTGCTGCTACCCTTAGTGCTGA |
| | | AAGGGTCAGAGGGGACAACAAAGAGTACGAATATAGTGTCGAATGCCAGGAAGAT |
| | | AGTGCGTGTCCGGCGGCAGAAGAGTCACTGCCAATTGAGGTGATGGTCGACGCTG |
| | | TGCACAAATTGAAATACGAGAATTATACCTCAAGTTTCTTCATCAGAGATATTAT |
| | | AAAGCCTGACCCGCCCAAAAATTTGCAACTCAAACCACTGAAAAATAGCCGCCAG |
| | | GTGGAAGTCTCATGGGAATATCCTGATACCTGGTCCACACCCCACTCCTATTTCT |
| | | CACTCACATTTTGCGTTCAGGTCCAGGGAAAGTCCAAGCGAGAAAAAAAAGATCG |
| | | CGTTTTCACGGACAAAACCTCAGCCACAGTGATTTGCCGCAAGAATGCTTCCATA |
| | | TCCGTACGCGCTCAAGACAGGTATTACTCATCTTCATGGTCTGAATGGGCCTCTG |
| | | TACCCTGTTCAGGAGGAGGTGGCAGTGGCGGGGGCGGATCAGGCGGTGGAGGTAG |
| | | CAGAAATTTGCCAGTGGCAACGCCAGATCCTGGTATGTTCCCGTGCCTCCACCAC |
| | | TCTCAGAACCTCTTGAGGGCTGTGTCCAACATGTTGCAAAAGGCGCGCCAAACGC |
| | | TCGAGTTTTACCCATGTACATCAGAGGAAATTGACCACGAGGACATTACGAAGGA |
| | | TAAAACCAGCACAGTAGAGGCATGTCTGCCATTGGAACTCACGAAAAACGAATCA |
| | | TGCCTTAACAGCCGAGAGACTTCTTTCATCACTAACGGATCTTGTCTTGCCTCAA |
| | | GAAAGACTTCATTCATGATGGCCCTCTGCCTCTCCTCAATCTACGAAGACCTCAA |
| | | AATGTACCAAGTTGAGTTCAAGACCATGAACGCTAAACTCCTTATGGATCCAAAG |
| | | CGCCAAATCTTTTTGGACCAAAACATGTTGGCTGTGATAGACGAGCTGATGCAGG |
| | | CTCTCAACTTCAATAGCGAGACCGTGCCCCAAAAGTCATCCCTTGAAGAACCAGA |
| | | TTTTTATAAAACGAAGATTAAATTGTGTATTCTGCTTCACGCTTTCCGGATCCGC |
| | | GCTGTGACCATTGATCGAGTTATGTCTTATCTGAACGCCTCTTAATAATAAAATC |
| | | GCTATCCATCGAAGATGGATGTGTGTTGGTTTTTTGTGTGCCAGCGTGAGTCTCT |
| | | CCTACCCTCCCGCTCTGGTCCTTCCTCTCCCGCTCTGCACCCTCTGTGGCCCTCG |
| | | CTGTGCTCTCTCGCTCCGTGACTTCCCTTCTCCAAGTTCTCCTTGGTGGCCCGCC |
| | | GTGGGGCTAGTCCAGGGCTGGATCTCGGGGAAGCGGCGGGGTGGCCTGGGAGTGG |
| | | GGAAGGGGGTGCGCACCCGGGACGCGCGCTACTTGCCCCTTTCGGCGGGGAGCAG |
| | | GGGAGACCTTTGGCCTACGGCGACGGGAGGGTCGGGACAAAGTTTAGGGCGTCGA |
| | | TAAGCGTCAGAGCGCCGAGGTTGGGGGAGGGTTTCTCTTCCGCTCTTTCGCGGGG |
| | | CCTCTGGCTCCCCCAGCGCAGCTGGAGTGGGGGACGGGTAGGCTCGTCCCAAAGG |
| | | CGCGGCGCTGAGGTTTGTGAACGCGTGGAGGGGCGCTTGGGGTCTGGGGGAGGCG |
| | | TCGCCCGGGTAAGCCTGTCTGCTGCGGCTCTGCTTCCCTTAGACTGGAGAGCTGT |
| | | GGACTTCGTCTAGGCGCCCGCTAAGTTCGCATGTCCTAGCACCTCTGGGTCTATG |
| | | TGGGGCCACACCGTGGGGAGGAAACAGCACGCGACGTTTGTAGAATGCTTGGCTG |
| | | TGATACAAAGCGGTTTCGAATAATTAACTTATTTGTTCCCATCACATGTCACTTT |
| | | TAAAAAATTATAAGAACTACCCGTTATTGACATCTTTCTGTGTGCCAAGGACTTT |
| | | ATGTGCTTTGCGTCATTTAATTTTGAAAACAGTTATCTTCCGCCATAGATAACTA |
| | | CTATGGTTATCTTCT |
| CTX1566* | 96 | GTTCTAGGGTGGAAACTAAGAGAATGATGTACCTAGAGGGCGCTGGAAGCTCTAA |
| | | AGCCCTAGCAGTTACTGCTTTTACTATTAGTGGTCGTTTTTTTCTCCCCCCCGCC |
| | | CCCCGACAAATCAACAGAACAAAGAAAATTACCTAAACAGCAAGGACATAGGGAG |
| | | GAACTTCTTGGCACAGAACTTTCCAAACACTTTTTCCTGAAGGGATACAAGAAGC |
| | | AAGAAAGGTACTCTTTCACTAGGACCTTCTCTGAGCTGTCCTCAGGATGCTTTTG |
| | | GGACTATTTTTCTTACCCAGAGAATGGAGAAACCCTGCAGGGAATTCCCAAGCTG |
| | | TAGTTATAAACAGAAGTTCTCCTTCTGCTAGGTAGCATTCAAAGATCTTAATCTT |
| | | CTGGGTTTCCGTTTTCTCGAATGAAAAATGCAGGTCCGAGCAGTTAACTGGCTGG |
| | | GGCACCATTAGCAAGTCACTTAGCATCTCTGGGGCCAGTCTGCAAAGCGAGGGGG |
| | | CAGCCTTAATGTGCCTCCAGCCTGAAGTCCTAGAATGAGCGCCCGGTGTCCCAAG |
| | | CTGGGGCGCGCACCCCAGATCGGAGGGCGCCGATGTACAGACAGCAAACTCACCC |
| | | AGTCTAGTGCATGCCTTCTTAAACATCACGAGACTCTAAGAAAAGGAAACTGAAA |
| | | ACGGGAAAGTCCCTCTCTCTAACCTGGCACTGCGTCGCTGGCTTGGAGACAGGTG |
| | | ACGGTCCCTGCGGGCCTTGTCCTGATTGGCTGGGCACGCGTTTAATATAAGTGGA |
| | | GGCGTCGCGCTGGCGGGCATTCCTGAAGCTTCATTTTGACACCCCCATAATATTT |
| | | TTCCAGAATTAACAGTATAAATTGCATCTCTTGTTCAAGAGTTCCCTATCACTCT |
| | | CTTTAATCACTACTCACAGTAACCTCAACTCCTGCAAGGCCAGCCCAGCACCAGC |
| | | ACCAGCCAACTCTCACTGAAGCCAGCTCTCTCTTCCTCCACCACCATGTGCCCAG |
| | | CCCGGAGTCTTCTGCTGGTAGCAACATTGGTTCTCCTGGACCATTTGTCACTGGC |
| | | AAGAAACCTGCCGGTAGCAACCCCCGATCCTGGTATGTTCCCTTGTTTGCATCAC |
| | | TCACAAAACCTTCTCCGCGCCGTTTCTAATATGCTGCAAAAGGCACGGCAGACCC |
| | | TTGAATTTTACCCGTGTACATCCGAAGAAATCGACCATGAAGACATTACCAAGGA |
| | | TAAGACCTCCACGGTGGAAGCTTGTCTCCCTTTGGAACTTACCAAGAATGAAAGC |
| | | TGCCTTAACTCTCGAGAGACTTCTTTCATCACTAATGGAAGCTGCCTGGCGTCCC |
| | | GGAAAACGTCCTTCATGATGGCGCTTTGTCTCTCCTCCATCTACGAGGATCTCAA |
| | | AATGTACCAGGTGGAATTTAAGACGATGAACGCAAAGCTTCTGATGGATCCCAAG |
| | | AGACAGATATTTCTGGACCAAAACATGTTGGCTGTCATCGACGAACTCATGCAGG |
| | | CTTTGAATTTTAACTCCGAGACGGTGCCACAGAAGTCCTCCCTCGAAGAACCGGA |
| | | TTTCTATAAGACTAAAATTAAATTGTGCATCCTGTTGCACGCGTTTCGCATTCGG |
| | | GCCGTCACAATTGACAGAGTAATGAGTTACCTGAACGCCTCAGGTGGGGGTGGCT |
| | | CCGGTGGAGGAGGATCAGGCGGTGGTGGCAGTATTTGGGAATTGAAAAAGGATGT |
| | | CTATGTTGTAGAACTTGATTGGTATCCGGACGCTCCAGGTGAAATGGTCGTTCTG |
| | | ACGTGCGATACACCTGAGGAAGATGGGATCACATGGACACTCGACCAGAGCTCTG |
| | | AGGTCCTCGGTAGCGGCAAGACGCTCACAATCCAGGTTAAGGAGTTCGGGGACGC |
| | | GGGGCAGTATACTTGCCATAAGGGCGGGGAAGTGCTCTCTCATAGCCTGCTCCTT |
| | | CTGCACAAGAAGGAAGATGGGATATGGTCCACGGACATCCTTAAAGACCAAAAGG |
| | | AGCCAAAGAATAAAACGTTTCTCAGGTGTGAAGCGAAAAACTATTCTGGGAGGTT |
| | | TACCTGTTGGTGGCTCACGACGATCTCCACAGACTTGACATTCAGTGTTAAATCT |
| | | AGCAGGGGATCATCTGACCCACAGGGAGTAACTTGTGGGGCCGCAACTCTCTCAG |
| | | CCGAGAGAGTGAGAGGGGACAATAAAGAGTACGAATATTCAGTAGAGTGCCAAGA |
| | | GGACAGCGCCTGCCCCGCTGCGGAAGAAAGTCTGCCGATTGAAGTCATGGTCGAC |
| | | GCCGTCCATAAGTTGAAGTACGAAAATTACACGTCTTCTTTTTTTATTCGAGACA |
| | | TAATAAAACCAGACCCCCCAAAAAATCTCCAACTGAAGCCCTTGAAAAACTCACG |
| | | CCAGGTTGAAGTGAGCTGGGAATATCCCGACACCTGGTCCACGCCGCATTCTTAT |
| | | TTTAGCTTGACGTTTTGTGTACAGGTTCAGGGTAAGAGTAAACGAGAAAAAAAAG |
| | | ACCGAGTTTTTACAGACAAGACTTCTGCCACAGTCATCTGCAGAAAAAATGCAAG |
| | | TATCAGTGTAAGAGCGCAGGACCGCTACTACTCTTCCTCTTGGAGCGAGTGGGCG |
| | | TCAGTTCCTTGCAGCTAATAATAAAATCGCTATCCATCGAAGATGGATGTGTGTT |
| | | GGTTTTTTGTGTGCCAGCGTGAGTCTCTCCTACCCTCCCGCTCTGGTCCTTCCTC |
| | | TCCCGCTCTGCACCCTCTGTGGCCCTCGCTGTGCTCTCTCGCTCCGTGACTTCCC |
| | | TTCTCCAAGTTCTCCTTGGTGGCCCGCCGTGGGGCTAGTCCAGGGCTGGATCTCG |
| | | GGGAAGCGGCGGGGTGGCCTGGGAGTGGGGAAGGGGGTGCGCACCCGGGACGCGC |
| | | GCTACTTGCCCCTTTCGGCGGGGAGCAGGGGAGACCTTTGGCCTACGGCGACGGG |
| | | AGGGTCGGGACAAAGTTTAGGGCGTCGATAAGCGTCAGAGCGCCGAGGTTGGGGG |
| | | AGGGTTTCTCTTCCGCTCTTTCGCGGGGCCTCTGGCTCCCCCAGCGCAGCTGGAG |
| | | TGGGGGACGGGTAGGCTCGTCCCAAAGGCGCGGCGCTGAGGTTTGTGAACGCGTG |
| | | GAGGGGCGCTTGGGGTCTGGGGGAGGCGTCGCCCGGGTAAGCCTGTCTGCTGCGG |
| | | CTCTGCTTCCCTTAGACTGGAGAGCTGTGGACTTCGTCTAGGCGCCCGCTAAGTT |
| | | CGCATGTCCTAGCACCTCTGGGTCTATGTGGGGCCACACCGTGGGGAGGAAACAG |
| | | CACGCGACGTTTGTAGAATGCTTGGCTGTGATACAAAGCGGTTTCGAATAATTAA |
| | | CTTATTTGTTCCCATCACATGTCACTTTTAAAAAATTATAAGAACTACCCGTTAT |
| | | TGACATCTTTCTGTGTGCCAAGGACTTTATGTGCTTTGCGTCATTTAATTTTGAA |
| | | AACAGTTATCTTCCGCCATAGATAACTACTATGGTTATCTTCT |
| CTX1567 | 97 | GTTCTAGGGTGGAAACTAAGAGAATGATGTACCTAGAGGGCGCTGGAAGCTCTAA |
| | | AGCCCTAGCAGTTACTGCTTTTACTATTAGTGGTCGTTTTTTTCTCCCCCCCGCC |
| | | CCCCGACAAATCAACAGAACAAAGAAAATTACCTAAACAGCAAGGACATAGGGAG |
| | | GAACTTCTTGGCACAGAACTTTCCAAACACTTTTTCCTGAAGGGATACAAGAAGC |
| | | AAGAAAGGTACTCTTTCACTAGGACCTTCTCTGAGCTGTCCTCAGGATGCTTTTG |
| | | GGACTATTTTTCTTACCCAGAGAATGGAGAAACCCTGCAGGGAATTCCCAAGCTG |
| | | TAGTTATAAACAGAAGTTCTCCTTCTGCTAGGTAGCATTCAAAGATCTTAATCTT |
| | | CTGGGTTTCCGTTTTCTCGAATGAAAAATGCAGGTCCGAGCAGTTAACTGGCTGG |
| | | GGCACCATTAGCAAGTCACTTAGCATCTCTGGGGCCAGTCTGCAAAGCGAGGGGG |
| | | CAGCCTTAATGTGCCTCCAGCCTGAAGTCCTAGAATGAGCGCCCGGTGTCCCAAG |
| | | CTGGGGCGCGCACCCCAGATCGGAGGGCGCCGATGTACAGACAGCAAACTCACCC |
| | | AGTCTAGTGCATGCCTTCTTAAACATCACGAGACTCTAAGAAAAGGAAACTGAAA |
| | | ACGGGAAAGTCCCTCTCTCTAACCTGGCACTGCGTCGCTGGCTTGGAGACAGGTG |
| | | ACGGTCCCTGCGGGCCTTGTCCTGATTGGCTGGGCACGCGTTTAATATAAGTGGA |
| | | GGCGTCGCGCTGGCGGGCATTCCTGAAGCTTACGCGTGCTAGCCCGGGCACTGAC |
| | | TCATCAAGCACTGACTCATCAAGCACTGACTCATCAAGGGACTCAGGGAGGGAAA |
| | | CTCCATTTTGACACCCCCATAATATTTTTCCAGAATTAACAGTATAAATTGCATC |
| | | TCTTGTTCAAGAGTTCCCTATCACTCTCTTTAATCACTACTCACAGTAACCTCAA |
| | | CTCCTGCAAGGCCAGCCCAGCACCAGCACCAGCCAACTCTCACTGAAGCCAGCTC |
| | | TCTCTTCCTCCACCACCATGTGCCCAGCCCGGAGTCTTCTGCTGGTAGCAACATT |
| | | GGTTCTCCTGGACCATTTGTCACTGGCAAGAAACCTGCCGGTAGCAACCCCCGAT |
| | | CCTGGTATGTTCCCTTGTTTGCATCACTCACAAAACCTTCTCCGCGCCGTTTCTA |
| | | ATATGCTGCAAAAGGCACGGCAGACCCTTGAATTTTACCCGTGTACATCCGAAGA |
| | | AATCGACCATGAAGACATTACCAAGGATAAGACCTCCACGGTGGAAGCTTGTCTC |
| | | CCTTTGGAACTTACCAAGAATGAAAGCTGCCTTAACTCTCGAGAGACTTCTTTCA |
| | | TCACTAATGGAAGCTGCCTGGCGTCCCGGAAAACGTCCTTCATGATGGCGCTTTG |
| | | TCTCTCCTCCATCTACGAGGATCTCAAAATGTACCAGGTGGAATTTAAGACGATG |
| | | AACGCAAAGCTTCTGATGGATCCCAAGAGACAGATATTTCTGGACCAAAACATGT |
| | | TGGCTGTCATCGACGAACTCATGCAGGCTTTGAATTTTAACTCCGAGACGGTGCC |
| | | ACAGAAGTCCTCCCTCGAAGAACCGGATTTCTATAAGACTAAAATTAAATTGTGC |
| | | ATCCTGTTGCACGCGTTTCGCATTCGGGCCGTCACAATTGACAGAGTAATGAGTT |
| | | ACCTGAACGCCTCAGGTGGGGGTGGCTCCGGTGGAGGAGGATCAGGCGGTGGTGG |
| | | CAGTATTTGGGAATTGAAAAAGGATGTCTATGTTGTAGAACTTGATTGGTATCCG |
| | | GACGCTCCAGGTGAAATGGTCGTTCTGACGTGCGATACACCTGAGGAAGATGGGA |
| | | TCACATGGACACTCGACCAGAGCTCTGAGGTCCTCGGTAGCGGCAAGACGCTCAC |
| | | AATCCAGGTTAAGGAGTTCGGGGACGCGGGGCAGTATACTTGCCATAAGGGCGGG |
| | | GAAGTGCTCTCTCATAGCCTGCTCCTTCTGCACAAGAAGGAAGATGGGATATGGT |
| | | CCACGGACATCCTTAAAGACCAAAAGGAGCCAAAGAATAAAACGTTTCTCAGGTG |
| | | TGAAGCGAAAAACTATTCTGGGAGGTTTACCTGTTGGTGGCTCACGACGATCTCC |
| | | ACAGACTTGACATTCAGTGTTAAATCTAGCAGGGGATCATCTGACCCACAGGGAG |
| | | TAACTTGTGGGGCCGCAACTCTCTCAGCCGAGAGAGTGAGAGGGGACAATAAAGA |
| | | GTACGAATATTCAGTAGAGTGCCAAGAGGACAGCGCCTGCCCCGCTGCGGAAGAA |
| | | AGTCTGCCGATTGAAGTCATGGTCGACGCCGTCCATAAGTTGAAGTACGAAAATT |
| | | ACACGTCTTCTTTTTTTATTCGAGACATAATAAAACCAGACCCCCCAAAAAATCT |
| | | CCAACTGAAGCCCTTGAAAAACTCACGCCAGGTTGAAGTGAGCTGGGAATATCCC |
| | | GACACCTGGTCCACGCCGCATTCTTATTTTAGCTTGACGTTTTGTGTACAGGTTC |
| | | AGGGTAAGAGTAAACGAGAAAAAAAAGACCGAGTTTTTACAGACAAGACTTCTGC |
| | | CACAGTCATCTGCAGAAAAAATGCAAGTATCAGTGTAAGAGCGCAGGACCGCTAC |
| | | TACTCTTCCTCTTGGAGCGAGTGGGCGTCAGTTCCTTGCAGCTAATAATAAAATC |
| | | GCTATCCATCGAAGATGGATGTGTGTTGGTTTTTTGTGTGCCAGCGTGAGTCTCT |
| | | CCTACCCTCCCGCTCTGGTCCTTCCTCTCCCGCTCTGCACCCTCTGTGGCCCTCG |
| | | CTGTGCTCTCTCGCTCCGTGACTTCCCTTCTCCAAGTTCTCCTTGGTGGCCCGCC |
| | | GTGGGGCTAGTCCAGGGCTGGATCTCGGGGAAGCGGCGGGGTGGCCTGGGAGTGG |
| | | GGAAGGGGGTGCGCACCCGGGACGCGCGCTACTTGCCCCTTTCGGCGGGGAGCAG |
| | | GGGAGACCTTTGGCCTACGGCGACGGGAGGGTCGGGACAAAGTTTAGGGCGTCGA |
| | | TAAGCGTCAGAGCGCCGAGGTTGGGGGAGGGTTTCTCTTCCGCTCTTTCGCGGGG |
| | | CCTCTGGCTCCCCCAGCGCAGCTGGAGTGGGGGACGGGTAGGCTCGTCCCAAAGG |
| | | CGCGGCGCTGAGGTTTGTGAACGCGTGGAGGGGCGCTTGGGGTCTGGGGGAGGCG |
| | | TCGCCCGGGTAAGCCTGTCTGCTGCGGCTCTGCTTCCCTTAGACTGGAGAGCTGT |
| | | GGACTTCGTCTAGGCGCCCGCTAAGTTCGCATGTCCTAGCACCTCTGGGTCTATG |
| | | TGGGGCCACACCGTGGGGAGGAAACAGCACGCGACGTTTGTAGAATGCTTGGCTG |
| | | TGATACAAAGCGGTTTCGAATAATTAACTTATTTGTTCCCATCACATGTCACTTT |
| | | TAAAAAATTATAAGAACTACCCGTTATTGACATCTTTCTGTGTGCCAAGGACTTT |
| | | ATGTGCTTTGCGTCATTTAATTTTGAAAACAGTTATCTTCCGCCATAGATAACTA |
| | | CTATGGTTATCTTCT |
| CTX1568* | 98 | GTTCAAGTCCAGCCTGGCCAACATGGTGAAACCCCATCTCTACTAAAAATACAAA |
| | | AAATTAGCCAGGCATGGTGGCGCGCGCATGTTACTCCCAGCTACTCGCGAGGCTC |
| | | AGACAGGAGAATCGCTTGAACCCAGGAGATCGAGGTTGCGGCGAGCTGAGATGGC |
| | | GCCACTGCACTCCAGCCTGGGTGACAGAGGGAGACCTCCGTCTCAAAAACAAAAC |
| | | AAATCAAAAAAATGCAGGAGAGGGGTACACGAATATTTGGGGAGCACCCCCAATT |
| | | CTTGGATGTCTGCTGTATCCCCAGTGCACAGCACAATCTAATCCCTAATAAATGT |
| | | GCAGTGGAGGTTTGTTGAATAAATGAATGGGCCCCAGAAGAATGAGGTGGAGAGG |
| | | GGAATAGGAAGATTGAATGTCTCCTGCCTGAAGGTCGGGCGGGGAGGGGTTGGGG |
| | | GCAGGCAACTCTGAGGCTCACCCGGGGCCACTGCCTGCATCCTGGCAACTGCCTC |
| | | CACCCACTTTAGGATCTTCAGACTGGCAGCGGTTGGAGGGAATTTCCCCTCGCCA |
| | | ATTGCTCAAGTCCCTCCCCTCGACCGGCCGGACATCCCCAGAGAGGGGCAGGCTG |
| | | GTCCCCTGACAGGTTGAAGCAAGTAGACGCCCAGGAGCCCCGGGAGGGGGCTGCA |
| | | GTTTCCTTCCTTCCTTCTCGGCAGCGCTCCGCGCCCCCATCGCCCCTCCTGCGCT |
| | | AGCGGAGGTGATCGCCGCGGCGATGCCGGAGGAGGGTTCGGGCTGCTCGGTGCGG |
| | | CGCAGGCCCTATGGGTGCGTCCTGCGGGCTCATTTTGACACCCCCATAATATTTT |
| | | TCCAGAATTAACAGTATAAATTGCATCTCTTGTTCAAGAGTTCCCTATCACTCTC |
| | | TTTAATCACTACTCACAGTAACCTCAACTCCTGCAAGGCCAGCCCAGCACCAGCA |
| | | CCAGCCAACTCTCACTGAAGCCAGCTCTCTCTTCCTCCACCACCATGTGTCATCA |
| | | GCAACTGGTCATCTCATGGTTTTCCCTGGTGTTTTTGGCGTCACCACTGGTGGCA |
| | | ATATGGGAGCTTAAGAAGGACGTCTACGTTGTCGAGCTGGATTGGTACCCAGACG |
| | | CTCCAGGAGAAATGGTCGTTCTGACGTGTGACACACCTGAGGAAGATGGTATTAC |
| | | CTGGACGCTTGATCAGTCATCCGAAGTTCTTGGGTCCGGGAAGACCCTTACAATC |
| | | CAGGTCAAAGAGTTCGGAGATGCTGGTCAGTATACTTGCCACAAGGGCGGTGAGG |
| | | TCCTTAGTCACAGTTTGCTTCTGCTCCACAAGAAGGAGGACGGCATATGGAGCAC |
| | | AGATATATTGAAAGACCAAAAAGAACCCAAAAATAAGACATTCCTTCGCTGCGAG |
| | | GCCAAGAACTACAGCGGCCGGTTTACGTGCTGGTGGCTCACAACCATATCCACAG |
| | | ATCTGACGTTCAGTGTTAAATCCTCAAGGGGTAGTAGCGATCCGCAAGGGGTTAC |
| | | GTGCGGTGCTGCTACCCTTAGTGCTGAAAGGGTCAGAGGGGACAACAAAGAGTAC |
| | | GAATATAGTGTCGAATGCCAGGAAGATAGTGCGTGTCCGGCGGCAGAAGAGTCAC |
| | | TGCCAATTGAGGTGATGGTCGACGCTGTGCACAAATTGAAATACGAGAATTATAC |
| | | CTCAAGTTTCTTCATCAGAGATATTATAAAGCCTGACCCGCCCAAAAATTTGCAA |
| | | CTCAAACCACTGAAAAATAGCCGCCAGGTGGAAGTCTCATGGGAATATCCTGATA |
| | | CCTGGTCCACACCCCACTCCTATTTCTCACTCACATTTTGCGTTCAGGTCCAGGG |
| | | AAAGTCCAAGCGAGAAAAAAAAGATCGCGTTTTCACGGACAAAACCTCAGCCACA |
| | | GTGATTTGCCGCAAGAATGCTTCCATATCCGTACGCGCTCAAGACAGGTATTACT |
| | | CATCTTCATGGTCTGAATGGGCCTCTGTACCCTGTTCAGGAGGAGGTGGCAGTGG |
| | | CGGGGGCGGATCAGGCGGTGGAGGTAGCAGAAATTTGCCAGTGGCAACGCCAGAT |
| | | CCTGGTATGTTCCCGTGCCTCCACCACTCTCAGAACCTCTTGAGGGCTGTGTCCA |
| | | ACATGTTGCAAAAGGCGCGCCAAACGCTCGAGTTTTACCCATGTACATCAGAGGA |
| | | AATTGACCACGAGGACATTACGAAGGATAAAACCAGCACAGTAGAGGCATGTCTG |
| | | CCATTGGAACTCACGAAAAACGAATCATGCCTTAACAGCCGAGAGACTTCTTTCA |
| | | TCACTAACGGATCTTGTCTTGCCTCAAGAAAGACTTCATTCATGATGGCCCTCTG |
| | | CCTCTCCTCAATCTACGAAGACCTCAAAATGTACCAAGTTGAGTTCAAGACCATG |
| | | AACGCTAAACTCCTTATGGATCCAAAGCGCCAAATCTTTTTGGACCAAAACATGT |
| | | TGGCTGTGATAGACGAGCTGATGCAGGCTCTCAACTTCAATAGCGAGACCGTGCC |
| | | CCAAAAGTCATCCCTTGAAGAACCAGATTTTTATAAAACGAAGATTAAATTGTGT |
| | | ATTCTGCTTCACGCTTTCCGGATCCGCGCTGTGACCATTGATCGAGTTATGTCTT |
| | | ATCTGAACGCCTCTTAATAATAAAATCGCTATCCATCGAAGATGGATGTGTGTTG |
| | | GTTTTTTGTGTGGGGCTTGGTGATCTGCCTCGTGGTGTGCATCCAGCGCTTCGCA |
| | | CAGGCTCAGCAGCAGCTGCCGCTCGAGTCACTTGGGGTGAGTTGAGATGGAAAAG |
| | | TTGGGAAGAAAACATAGAGAGGCGCGTGACCGAAAAGACAGAATGAGATGGGTAC |
| | | AAAGAGGCCAGAGAGGAAGATCTGGTAGGGCAGAGACAGAGACCAGAACAGGGAG |
| | | GCGAGGCGGGGACCAGGCTGCCCGGTGTAGGGGCTACGAGACAGGCAGCCCTGCC |
| | | AGGAGGTACAGGGAGATCCCGGGATGGGAAAGGTAGGCACACATGGAAATGGAAG |
| | | ATGACTCGGCTCTGGTGTTCCCCCGGCAGGCTGACTCAGAGGCTGCTGGGGGCTT |
| | | CACAAGGCTGGGCGTGGGGGCTTCCTGGGGCCTCCTAGGACGGGATGGCCCCAGC |
| | | CACTCGCTCCGGGTGGGGGAGGGGTCCCTTTGGGGACCGCGCCGGGCGCCTTTGC |
| | | AGCGTAGAGAGTCCGCTGCGCGCGGTGCTCTCGCGCCCAGTGACATCCAGGAAAA |
| | | CGATTCGGGAAACGAAGAAGTTCTTTTGAAGGTCTCGACTTCACGTTCCCCGCTG |
| | | GTTCAGACCTGCTTCCTCTTTAAGAAGTCTTAAGAGTAAAAAAAAATAAAATGAA |
| | | ATAAAATCACCAGTGCGCGCCGTGGGATGAGAGGTGGAAAGGAGGATGGACAGAG |
| | | AAAAGAGAGCTCCTGGCACAGGGGACACATAGAACCTCTCTGCTTACGTCCGTGC |
| | | CCTGTTTTCTGGTCTTTTCTTCCAGTGGGACGTAGCTGAGCT |
| CTX1569 | 99 | GTTCAAGTCCAGCCTGGCCAACATGGTGAAACCCCATCTCTACTAAAAATACAAA |
| | | AAATTAGCCAGGCATGGTGGCGCGCGCATGTTACTCCCAGCTACTCGCGAGGCTC |
| | | AGACAGGAGAATCGCTTGAACCCAGGAGATCGAGGTTGCGGCGAGCTGAGATGGC |
| | | GCCACTGCACTCCAGCCTGGGTGACAGAGGGAGACCTCCGTCTCAAAAACAAAAC |
| | | AAATCAAAAAAATGCAGGAGAGGGGTACACGAATATTTGGGGAGCACCCCCAATT |
| | | CTTGGATGTCTGCTGTATCCCCAGTGCACAGCACAATCTAATCCCTAATAAATGT |
| | | GCAGTGGAGGTTTGTTGAATAAATGAATGGGCCCCAGAAGAATGAGGTGGAGAGG |
| | | GGAATAGGAAGATTGAATGTCTCCTGCCTGAAGGTCGGGCGGGGAGGGGTTGGGG |
| | | GCAGGCAACTCTGAGGCTCACCCGGGGCCACTGCCTGCATCCTGGCAACTGCCTC |
| | | CACCCACTTTAGGATCTTCAGACTGGCAGCGGTTGGAGGGAATTTCCCCTCGCCA |
| | | ATTGCTCAAGTCCCTCCCCTCGACCGGCCGGACATCCCCAGAGAGGGGCAGGCTG |
| | | GTCCCCTGACAGGTTGAAGCAAGTAGACGCCCAGGAGCCCCGGGAGGGGGCTGCA |
| | | GTTTCCTTCCTTCCTTCTCGGCAGCGCTCCGCGCCCCCATCGCCCCTCCTGCGCT |
| | | AGCGGAGGTGATCGCCGCGGCGATGCCGGAGGAGGGTTCGGGCTGCTCGGTGCGG |
| | | CGCAGGCCCTATGGGTGCGTCCTGCGGGCTACGCGTGCTAGCCCGGGCACTGACT |
| | | CATCAAGCACTGACTCATCAAGCACTGACTCATCAAGGGACTCAGGGAGGGAAAC |
| | | TCCATTTTGACACCCCCATAATATTTTTCCAGAATTAACAGTATAAATTGCATCT |
| | | CTTGTTCAAGAGTTCCCTATCACTCTCTTTAATCACTACTCACAGTAACCTCAAC |
| | | TCCTGCAAGGCCAGCCCAGCACCAGCACCAGCCAACTCTCACTGAAGCCAGCTCT |
| | | CTCTTCCTCCACCACCATGTGTCATCAGCAACTGGTCATCTCATGGTTTTCCCTG |
| | | GTGTTTTTGGCGTCACCACTGGTGGCAATATGGGAGCTTAAGAAGGACGTCTACG |
| | | TTGTCGAGCTGGATTGGTACCCAGACGCTCCAGGAGAAATGGTCGTTCTGACGTG |
| | | TGACACACCTGAGGAAGATGGTATTACCTGGACGCTTGATCAGTCATCCGAAGTT |
| | | CTTGGGTCCGGGAAGACCCTTACAATCCAGGTCAAAGAGTTCGGAGATGCTGGTC |
| | | AGTATACTTGCCACAAGGGCGGTGAGGTCCTTAGTCACAGTTTGCTTCTGCTCCA |
| | | CAAGAAGGAGGACGGCATATGGAGCACAGATATATTGAAAGACCAAAAAGAACCC |
| | | AAAAATAAGACATTCCTTCGCTGCGAGGCCAAGAACTACAGCGGCCGGTTTACGT |
| | | GCTGGTGGCTCACAACCATATCCACAGATCTGACGTTCAGTGTTAAATCCTCAAG |
| | | GGGTAGTAGCGATCCGCAAGGGGTTACGTGCGGTGCTGCTACCCTTAGTGCTGAA |
| | | AGGGTCAGAGGGGACAACAAAGAGTACGAATATAGTGTCGAATGCCAGGAAGATA |
| | | GTGCGTGTCCGGCGGCAGAAGAGTCACTGCCAATTGAGGTGATGGTCGACGCTGT |
| | | GCACAAATTGAAATACGAGAATTATACCTCAAGTTTCTTCATCAGAGATATTATA |
| | | AAGCCTGACCCGCCCAAAAATTTGCAACTCAAACCACTGAAAAATAGCCGCCAGG |
| | | TGGAAGTCTCATGGGAATATCCTGATACCTGGTCCACACCCCACTCCTATTTCTC |
| | | ACTCACATTTTGCGTTCAGGTCCAGGGAAAGTCCAAGCGAGAAAAAAAAGATCGC |
| | | GTTTTCACGGACAAAACCTCAGCCACAGTGATTTGCCGCAAGAATGCTTCCATAT |
| | | CCGTACGCGCTCAAGACAGGTATTACTCATCTTCATGGTCTGAATGGGCCTCTGT |
| | | ACCCTGTTCAGGAGGAGGTGGCAGTGGCGGGGGCGGATCAGGCGGTGGAGGTAGC |
| | | AGAAATTTGCCAGTGGCAACGCCAGATCCTGGTATGTTCCCGTGCCTCCACCACT |
| | | CTCAGAACCTCTTGAGGGCTGTGTCCAACATGTTGCAAAAGGCGCGCCAAACGCT |
| | | CGAGTTTTACCCATGTACATCAGAGGAAATTGACCACGAGGACATTACGAAGGAT |
| | | AAAACCAGCACAGTAGAGGCATGTCTGCCATTGGAACTCACGAAAAACGAATCAT |
| | | GCCTTAACAGCCGAGAGACTTCTTTCATCACTAACGGATCTTGTCTTGCCTCAAG |
| | | AAAGACTTCATTCATGATGGCCCTCTGCCTCTCCTCAATCTACGAAGACCTCAAA |
| | | ATGTACCAAGTTGAGTTCAAGACCATGAACGCTAAACTCCTTATGGATCCAAAGC |
| | | GCCAAATCTTTTTGGACCAAAACATGTTGGCTGTGATAGACGAGCTGATGCAGGC |
| | | TCTCAACTTCAATAGCGAGACCGTGCCCCAAAAGTCATCCCTTGAAGAACCAGAT |
| | | TTTTATAAAACGAAGATTAAATTGTGTATTCTGCTTCACGCTTTCCGGATCCGCG |
| | | CTGTGACCATTGATCGAGTTATGTCTTATCTGAACGCCTCTTAATAATAAAATCG |
| | | CTATCCATCGAAGATGGATGTGTGTTGGTTTTTTGTGTGGGGCTTGGTGATCTGC |
| | | CTCGTGGTGTGCATCCAGCGCTTCGCACAGGCTCAGCAGCAGCTGCCGCTCGAGT |
| | | CACTTGGGGTGAGTTGAGATGGAAAAGTTGGGAAGAAAACATAGAGAGGCGCGTG |
| | | ACCGAAAAGACAGAATGAGATGGGTACAAAGAGGCCAGAGAGGAAGATCTGGTAG |
| | | GGCAGAGACAGAGACCAGAACAGGGAGGCGAGGCGGGGACCAGGCTGCCCGGTGT |
| | | AGGGGCTACGAGACAGGCAGCCCTGCCAGGAGGTACAGGGAGATCCCGGGATGGG |
| | | AAAGGTAGGCACACATGGAAATGGAAGATGACTCGGCTCTGGTGTTCCCCCGGCA |
| | | GGCTGACTCAGAGGCTGCTGGGGGCTTCACAAGGCTGGGCGTGGGGGCTTCCTGG |
| | | GGCCTCCTAGGACGGGATGGCCCCAGCCACTCGCTCCGGGTGGGGGAGGGGTCCC |
| | | TTTGGGGACCGCGCCGGGCGCCTTTGCAGCGTAGAGAGTCCGCTGCGCGCGGTGC |
| | | TCTCGCGCCCAGTGACATCCAGGAAAACGATTCGGGAAACGAAGAAGTTCTTTTG |
| | | AAGGTCTCGACTTCACGTTCCCCGCTGGTTCAGACCTGCTTCCTCTTTAAGAAGT |
| | | CTTAAGAGTAAAAAAAAATAAAATGAAATAAAATCACCAGTGCGCGCCGTGGGAT |
| | | GAGAGGTGGAAAGGAGGATGGACAGAGAAAAGAGAGCTCCTGGCACAGGGGACAC |
| | | ATAGAACCTCTCTGCTTACGTCCGTGCCCTGTTTTCTGGTCTTTTCTTCCAGTGG |
| | | GACGTAGCTGAGCT |
| CTX1570* | 100 | GTTCAAGTCCAGCCTGGCCAACATGGTGAAACCCCATCTCTACTAAAAATACAAA |
| | | AAATTAGCCAGGCATGGTGGCGCGCGCATGTTACTCCCAGCTACTCGCGAGGCTC |
| | | AGACAGGAGAATCGCTTGAACCCAGGAGATCGAGGTTGCGGCGAGCTGAGATGGC |
| | | GCCACTGCACTCCAGCCTGGGTGACAGAGGGAGACCTCCGTCTCAAAAACAAAAC |
| | | AAATCAAAAAAATGCAGGAGAGGGGTACACGAATATTTGGGGAGCACCCCCAATT |
| | | CTTGGATGTCTGCTGTATCCCCAGTGCACAGCACAATCTAATCCCTAATAAATGT |
| | | GCAGTGGAGGTTTGTTGAATAAATGAATGGGCCCCAGAAGAATGAGGTGGAGAGG |
| | | GGAATAGGAAGATTGAATGTCTCCTGCCTGAAGGTCGGGCGGGGAGGGGTTGGGG |
| | | GCAGGCAACTCTGAGGCTCACCCGGGGCCACTGCCTGCATCCTGGCAACTGCCTC |
| | | CACCCACTTTAGGATCTTCAGACTGGCAGCGGTTGGAGGGAATTTCCCCTCGCCA |
| | | ATTGCTCAAGTCCCTCCCCTCGACCGGCCGGACATCCCCAGAGAGGGGCAGGCTG |
| | | GTCCCCTGACAGGTTGAAGCAAGTAGACGCCCAGGAGCCCCGGGAGGGGGCTGCA |
| | | GTTTCCTTCCTTCCTTCTCGGCAGCGCTCCGCGCCCCCATCGCCCCTCCTGCGCT |
| | | AGCGGAGGTGATCGCCGCGGCGATGCCGGAGGAGGGTTCGGGCTGCTCGGTGCGG |
| | | CGCAGGCCCTATGGGTGCGTCCTGCGGGCTCATTTTGACACCCCCATAATATTTT |
| | | TCCAGAATTAACAGTATAAATTGCATCTCTTGTTCAAGAGTTCCCTATCACTCTC |
| | | TTTAATCACTACTCACAGTAACCTCAACTCCTGCAAGGCCAGCCCAGCACCAGCA |
| | | CCAGCCAACTCTCACTGAAGCCAGCTCTCTCTTCCTCCACCACCATGTGCCCAGC |
| | | CCGGAGTCTTCTGCTGGTAGCAACATTGGTTCTCCTGGACCATTTGTCACTGGCA |
| | | AGAAACCTGCCGGTAGCAACCCCCGATCCTGGTATGTTCCCTTGTTTGCATCACT |
| | | CACAAAACCTTCTCCGCGCCGTTTCTAATATGCTGCAAAAGGCACGGCAGACCCT |
| | | TGAATTTTACCCGTGTACATCCGAAGAAATCGACCATGAAGACATTACCAAGGAT |
| | | AAGACCTCCACGGTGGAAGCTTGTCTCCCTTTGGAACTTACCAAGAATGAAAGCT |
| | | GCCTTAACTCTCGAGAGACTTCTTTCATCACTAATGGAAGCTGCCTGGCGTCCCG |
| | | GAAAACGTCCTTCATGATGGCGCTTTGTCTCTCCTCCATCTACGAGGATCTCAAA |
| | | ATGTACCAGGTGGAATTTAAGACGATGAACGCAAAGCTTCTGATGGATCCCAAGA |
| | | GACAGATATTTCTGGACCAAAACATGTTGGCTGTCATCGACGAACTCATGCAGGC |
| | | TTTGAATTTTAACTCCGAGACGGTGCCACAGAAGTCCTCCCTCGAAGAACCGGAT |
| | | TTCTATAAGACTAAAATTAAATTGTGCATCCTGTTGCACGCGTTTCGCATTCGGG |
| | | CCGTCACAATTGACAGAGTAATGAGTTACCTGAACGCCTCAGGTGGGGGTGGCTC |
| | | CGGTGGAGGAGGATCAGGCGGTGGTGGCAGTATTTGGGAATTGAAAAAGGATGTC |
| | | TATGTTGTAGAACTTGATTGGTATCCGGACGCTCCAGGTGAAATGGTCGTTCTGA |
| | | CGTGCGATACACCTGAGGAAGATGGGATCACATGGACACTCGACCAGAGCTCTGA |
| | | GGTCCTCGGTAGCGGCAAGACGCTCACAATCCAGGTTAAGGAGTTCGGGGACGCG |
| | | GGGCAGTATACTTGCCATAAGGGCGGGGAAGTGCTCTCTCATAGCCTGCTCCTTC |
| | | TGCACAAGAAGGAAGATGGGATATGGTCCACGGACATCCTTAAAGACCAAAAGGA |
| | | GCCAAAGAATAAAACGTTTCTCAGGTGTGAAGCGAAAAACTATTCTGGGAGGTTT |
| | | ACCTGTTGGTGGCTCACGACGATCTCCACAGACTTGACATTCAGTGTTAAATCTA |
| | | GCAGGGGATCATCTGACCCACAGGGAGTAACTTGTGGGGCCGCAACTCTCTCAGC |
| | | CGAGAGAGTGAGAGGGGACAATAAAGAGTACGAATATTCAGTAGAGTGCCAAGAG |
| | | GACAGCGCCTGCCCCGCTGCGGAAGAAAGTCTGCCGATTGAAGTCATGGTCGACG |
| | | CCGTCCATAAGTTGAAGTACGAAAATTACACGTCTTCTTTTTTTATTCGAGACAT |
| | | AATAAAACCAGACCCCCCAAAAAATCTCCAACTGAAGCCCTTGAAAAACTCACGC |
| | | CAGGTTGAAGTGAGCTGGGAATATCCCGACACCTGGTCCACGCCGCATTCTTATT |
| | | TTAGCTTGACGTTTTGTGTACAGGTTCAGGGTAAGAGTAAACGAGAAAAAAAAGA |
| | | CCGAGTTTTTACAGACAAGACTTCTGCCACAGTCATCTGCAGAAAAAATGCAAGT |
| | | ATCAGTGTAAGAGCGCAGGACCGCTACTACTCTTCCTCTTGGAGCGAGTGGGCGT |
| | | CAGTTCCTTGCAGCTAATAATAAAATCGCTATCCATCGAAGATGGATGTGTGTTG |
| | | GTTTTTTGTGTGGGGCTTGGTGATCTGCCTCGTGGTGTGCATCCAGCGCTTCGCA |
| | | CAGGCTCAGCAGCAGCTGCCGCTCGAGTCACTTGGGGTGAGTTGAGATGGAAAAG |
| | | TTGGGAAGAAAACATAGAGAGGCGCGTGACCGAAAAGACAGAATGAGATGGGTAC |
| | | AAAGAGGCCAGAGAGGAAGATCTGGTAGGGCAGAGACAGAGACCAGAACAGGGAG |
| | | GCGAGGCGGGGACCAGGCTGCCCGGTGTAGGGGCTACGAGACAGGCAGCCCTGCC |
| | | AGGAGGTACAGGGAGATCCCGGGATGGGAAAGGTAGGCACACATGGAAATGGAAG |
| | | ATGACTCGGCTCTGGTGTTCCCCCGGCAGGCTGACTCAGAGGCTGCTGGGGGCTT |
| | | CACAAGGCTGGGCGTGGGGGCTTCCTGGGGCCTCCTAGGACGGGATGGCCCCAGC |
| | | CACTCGCTCCGGGTGGGGGAGGGGTCCCTTTGGGGACCGCGCCGGGCGCCTTTGC |
| | | AGCGTAGAGAGTCCGCTGCGCGCGGTGCTCTCGCGCCCAGTGACATCCAGGAAAA |
| | | CGATTCGGGAAACGAAGAAGTTCTTTTGAAGGTCTCGACTTCACGTTCCCCGCTG |
| | | GTTCAGACCTGCTTCCTCTTTAAGAAGTCTTAAGAGTAAAAAAAAATAAAATGAA |
| | | ATAAAATCACCAGTGCGCGCCGTGGGATGAGAGGTGGAAAGGAGGATGGACAGAG |
| | | AAAAGAGAGCTCCTGGCACAGGGGACACATAGAACCTCTCTGCTTACGTCCGTGC |
| | | CCTGTTTTCTGGTCTTTTCTTCCAGTGGGACGTAGCTGAGCT |
| CTX1571 | 101 | GTTCAAGTCCAGCCTGGCCAACATGGTGAAACCCCATCTCTACTAAAAATACAAA |
| | | AAATTAGCCAGGCATGGTGGCGCGCGCATGTTACTCCCAGCTACTCGCGAGGCTC |
| | | AGACAGGAGAATCGCTTGAACCCAGGAGATCGAGGTTGCGGCGAGCTGAGATGGC |
| | | GCCACTGCACTCCAGCCTGGGTGACAGAGGGAGACCTCCGTCTCAAAAACAAAAC |
| | | AAATCAAAAAAATGCAGGAGAGGGGTACACGAATATTTGGGGAGCACCCCCAATT |
| | | CTTGGATGTCTGCTGTATCCCCAGTGCACAGCACAATCTAATCCCTAATAAATGT |
| | | GCAGTGGAGGTTTGTTGAATAAATGAATGGGCCCCAGAAGAATGAGGTGGAGAGG |
| | | GGAATAGGAAGATTGAATGTCTCCTGCCTGAAGGTCGGGCGGGGAGGGGTTGGGG |
| | | GCAGGCAACTCTGAGGCTCACCCGGGGCCACTGCCTGCATCCTGGCAACTGCCTC |
| | | CACCCACTTTAGGATCTTCAGACTGGCAGCGGTTGGAGGGAATTTCCCCTCGCCA |
| | | ATTGCTCAAGTCCCTCCCCTCGACCGGCCGGACATCCCCAGAGAGGGGCAGGCTG |
| | | GTCCCCTGACAGGTTGAAGCAAGTAGACGCCCAGGAGCCCCGGGAGGGGGCTGCA |
| | | GTTTCCTTCCTTCCTTCTCGGCAGCGCTCCGCGCCCCCATCGCCCCTCCTGCGCT |
| | | AGCGGAGGTGATCGCCGCGGCGATGCCGGAGGAGGGTTCGGGCTGCTCGGTGCGG |
| | | CGCAGGCCCTATGGGTGCGTCCTGCGGGCTACGCGTGCTAGCCCGGGCACTGACT |
| | | CATCAAGCACTGACTCATCAAGCACTGACTCATCAAGGGACTCAGGGAGGGAAAC |
| | | TCCATTTTGACACCCCCATAATATTTTTCCAGAATTAACAGTATAAATTGCATCT |
| | | CTTGTTCAAGAGTTCCCTATCACTCTCTTTAATCACTACTCACAGTAACCTCAAC |
| | | TCCTGCAAGGCCAGCCCAGCACCAGCACCAGCCAACTCTCACTGAAGCCAGCTCT |
| | | CTCTTCCTCCACCACCATGTGCCCAGCCCGGAGTCTTCTGCTGGTAGCAACATTG |
| | | GTTCTCCTGGACCATTTGTCACTGGCAAGAAACCTGCCGGTAGCAACCCCCGATC |
| | | CTGGTATGTTCCCTTGTTTGCATCACTCACAAAACCTTCTCCGCGCCGTTTCTAA |
| | | TATGCTGCAAAAGGCACGGCAGACCCTTGAATTTTACCCGTGTACATCCGAAGAA |
| | | ATCGACCATGAAGACATTACCAAGGATAAGACCTCCACGGTGGAAGCTTGTCTCC |
| | | CTTTGGAACTTACCAAGAATGAAAGCTGCCTTAACTCTCGAGAGACTTCTTTCAT |
| | | CACTAATGGAAGCTGCCTGGCGTCCCGGAAAACGTCCTTCATGATGGCGCTTTGT |
| | | CTCTCCTCCATCTACGAGGATCTCAAAATGTACCAGGTGGAATTTAAGACGATGA |
| | | ACGCAAAGCTTCTGATGGATCCCAAGAGACAGATATTTCTGGACCAAAACATGTT |
| | | GGCTGTCATCGACGAACTCATGCAGGCTTTGAATTTTAACTCCGAGACGGTGCCA |
| | | CAGAAGTCCTCCCTCGAAGAACCGGATTTCTATAAGACTAAAATTAAATTGTGCA |
| | | TCCTGTTGCACGCGTTTCGCATTCGGGCCGTCACAATTGACAGAGTAATGAGTTA |
| | | CCTGAACGCCTCAGGTGGGGGTGGCTCCGGTGGAGGAGGATCAGGCGGTGGTGGC |
| | | AGTATTTGGGAATTGAAAAAGGATGTCTATGTTGTAGAACTTGATTGGTATCCGG |
| | | ACGCTCCAGGTGAAATGGTCGTTCTGACGTGCGATACACCTGAGGAAGATGGGAT |
| | | CACATGGACACTCGACCAGAGCTCTGAGGTCCTCGGTAGCGGCAAGACGCTCACA |
| | | ATCCAGGTTAAGGAGTTCGGGGACGCGGGGCAGTATACTTGCCATAAGGGCGGGG |
| | | AAGTGCTCTCTCATAGCCTGCTCCTTCTGCACAAGAAGGAAGATGGGATATGGTC |
| | | CACGGACATCCTTAAAGACCAAAAGGAGCCAAAGAATAAAACGTTTCTCAGGTGT |
| | | GAAGCGAAAAACTATTCTGGGAGGTTTACCTGTTGGTGGCTCACGACGATCTCCA |
| | | CAGACTTGACATTCAGTGTTAAATCTAGCAGGGGATCATCTGACCCACAGGGAGT |
| | | AACTTGTGGGGCCGCAACTCTCTCAGCCGAGAGAGTGAGAGGGGACAATAAAGAG |
| | | TACGAATATTCAGTAGAGTGCCAAGAGGACAGCGCCTGCCCCGCTGCGGAAGAAA |
| | | GTCTGCCGATTGAAGTCATGGTCGACGCCGTCCATAAGTTGAAGTACGAAAATTA |
| | | CACGTCTTCTTTTTTTATTCGAGACATAATAAAACCAGACCCCCCAAAAAATCTC |
| | | CAACTGAAGCCCTTGAAAAACTCACGCCAGGTTGAAGTGAGCTGGGAATATCCCG |
| | | ACACCTGGTCCACGCCGCATTCTTATTTTAGCTTGACGTTTTGTGTACAGGTTCA |
| | | GGGTAAGAGTAAACGAGAAAAAAAAGACCGAGTTTTTACAGACAAGACTTCTGCC |
| | | ACAGTCATCTGCAGAAAAAATGCAAGTATCAGTGTAAGAGCGCAGGACCGCTACT |
| | | ACTCTTCCTCTTGGAGCGAGTGGGCGTCAGTTCCTTGCAGCTAATAATAAAATCG |
| | | CTATCCATCGAAGATGGATGTGTGTTGGTTTTTTGTGTGGGGCTTGGTGATCTGC |
| | | CTCGTGGTGTGCATCCAGCGCTTCGCACAGGCTCAGCAGCAGCTGCCGCTCGAGT |
| | | CACTTGGGGTGAGTTGAGATGGAAAAGTTGGGAAGAAAACATAGAGAGGCGCGTG |
| | | ACCGAAAAGACAGAATGAGATGGGTACAAAGAGGCCAGAGAGGAAGATCTGGTAG |
| | | GGCAGAGACAGAGACCAGAACAGGGAGGCGAGGCGGGGACCAGGCTGCCCGGTGT |
| | | AGGGGCTACGAGACAGGCAGCCCTGCCAGGAGGTACAGGGAGATCCCGGGATGGG |
| | | AAAGGTAGGCACACATGGAAATGGAAGATGACTCGGCTCTGGTGTTCCCCCGGCA |
| | | GGCTGACTCAGAGGCTGCTGGGGGCTTCACAAGGCTGGGCGTGGGGGCTTCCTGG |
| | | GGCCTCCTAGGACGGGATGGCCCCAGCCACTCGCTCCGGGTGGGGGAGGGGTCCC |
| | | TTTGGGGACCGCGCCGGGCGCCTTTGCAGCGTAGAGAGTCCGCTGCGCGCGGTGC |
| | | TCTCGCGCCCAGTGACATCCAGGAAAACGATTCGGGAAACGAAGAAGTTCTTTTG |
| | | AAGGTCTCGACTTCACGTTCCCCGCTGGTTCAGACCTGCTTCCTCTTTAAGAAGT |
| | | CTTAAGAGTAAAAAAAAATAAAATGAAATAAAATCACCAGTGCGCGCCGTGGGAT |
| | | GAGAGGTGGAAAGGAGGATGGACAGAGAAAAGAGAGCTCCTGGCACAGGGGACAC |
| | | ATAGAACCTCTCTGCTTACGTCCGTGCCCTGTTTTCTGGTCTTTTCTTCCAGTGG |
| | | GACGTAGCTGAGCT |
| 3XAP-Min | 102 | ACGCGTGCTAGCCCGGGCACTGACTCATCAAGCACTGACTCATCAAGCACTGACT |
| IL2 | | CATCAAGGGACTCAGGGAGGGAAACTCCATTTTGACACCCCCATAATATTTTTCC |
| promoter- | | AGAATTAACAGTATAAATTGCATCTCTTGTTCAAGAGTTCCCTATCACTCTCTTT |
| hIL12 (901) | | AATCACTACTCACAGTAACCTCAACTCCTGCAAGGCCAGCCCAGCACCAGCACCA |
| expression | | GCCAACTCTCACTGAAGCCAGCTCTCTCTTCCTCCACCACCATGTGCCCAGCCCG |
| cassette | | GAGTCTTCTGCTGGTAGCAACATTGGTTCTCCTGGACCATTTGTCACTGGCAAGA |
| | | AACCTGCCGGTAGCAACCCCCGATCCTGGTATGTTCCCTTGTTTGCATCACTCAC |
| | | AAAACCTTCTCCGCGCCGTTTCTAATATGCTGCAAAAGGCACGGCAGACCCTTGA |
| | | ATTTTACCCGTGTACATCCGAAGAAATCGACCATGAAGACATTACCAAGGATAAG |
| | | ACCTCCACGGTGGAAGCTTGTCTCCCTTTGGAACTTACCAAGAATGAAAGCTGCC |
| | | TTAACTCTCGAGAGACTTCTTTCATCACTAATGGAAGCTGCCTGGCGTCCCGGAA |
| | | AACGTCCTTCATGATGGCGCTTTGTCTCTCCTCCATCTACGAGGATCTCAAAATG |
| | | TACCAGGTGGAATTTAAGACGATGAACGCAAAGCTTCTGATGGATCCCAAGAGAC |
| | | AGATATTTCTGGACCAAAACATGTTGGCTGTCATCGACGAACTCATGCAGGCTTT |
| | | GAATTTTAACTCCGAGACGGTGCCACAGAAGTCCTCCCTCGAAGAACCGGATTTC |
| | | TATAAGACTAAAATTAAATTGTGCATCCTGTTGCACGCGTTTCGCATTCGGGCCG |
| | | TCACAATTGACAGAGTAATGAGTTACCTGAACGCCTCAGGTGGGGGTGGCTCCGG |
| | | TGGAGGAGGATCAGGCGGTGGTGGCAGTATTTGGGAATTGAAAAAGGATGTCTAT |
| | | GTTGTAGAACTTGATTGGTATCCGGACGCTCCAGGTGAAATGGTCGTTCTGACGT |
| | | GCGATACACCTGAGGAAGATGGGATCACATGGACACTCGACCAGAGCTCTGAGGT |
| | | CCTCGGTAGCGGCAAGACGCTCACAATCCAGGTTAAGGAGTTCGGGGACGCGGGG |
| | | CAGTATACTTGCCATAAGGGCGGGGAAGTGCTCTCTCATAGCCTGCTCCTTCTGC |
| | | ACAAGAAGGAAGATGGGATATGGTCCACGGACATCCTTAAAGACCAAAAGGAGCC |
| | | AAAGAATAAAACGTTTCTCAGGTGTGAAGCGAAAAACTATTCTGGGAGGTTTACC |
| | | TGTTGGTGGCTCACGACGATCTCCACAGACTTGACATTCAGTGTTAAATCTAGCA |
| | | GGGGATCATCTGACCCACAGGGAGTAACTTGTGGGGCCGCAACTCTCTCAGCCGA |
| | | GAGAGTGAGAGGGGACAATAAAGAGTACGAATATTCAGTAGAGTGCCAAGAGGAC |
| | | AGCGCCTGCCCCGCTGCGGAAGAAAGTCTGCCGATTGAAGTCATGGTCGACGCCG |
| | | TCCATAAGTTGAAGTACGAAAATTACACGTCTTCTTTTTTTATTCGAGACATAAT |
| | | AAAACCAGACCCCCCAAAAAATCTCCAACTGAAGCCCTTGAAAAACTCACGCCAG |
| | | GTTGAAGTGAGCTGGGAATATCCCGACACCTGGTCCACGCCGCATTCTTATTTTA |
| | | GCTTGACGTTTTGTGTACAGGTTCAGGGTAAGAGTAAACGAGAAAAAAAAGACCG |
| | | AGTTTTTACAGACAAGACTTCTGCCACAGTCATCTGCAGAAAAAATGCAAGTATC |
| | | AGTGTAAGAGCGCAGGACCGCTACTACTCTTCCTCTTGGAGCGAGTGGGCGTCAG |
| | | TTCCTTGCAGCTAATAATAAAATCGCTATCCATCGAAGATGGATGTGTGTTGGTT |
| | | TTTTGTGTG |
| 3XAP-Min | 103 | ACGCGTGCTAGCCCGGGCACTGACTCATCAAGCACTGACTCATCAAGCACTGACT |
| IL2 | | CATCAAGGGACTCAGGGAGGGAAACTCCATTTTGACACCCCCATAATATTTTTCC |
| promoter- | | AGAATTAACAGTATAAATTGCATCTCTTGTTCAAGAGTTCCCTATCACTCTCTTT |
| hIL12 (902) | | AATCACTACTCACAGTAACCTCAACTCCTGCAAGGCCAGCCCAGCACCAGCACCA |
| expression | | GCCAACTCTCACTGAAGCCAGCTCTCTCTTCCTCCACCACCATGTGTCATCAGCA |
| cassette | | ACTGGTCATCTCATGGTTTTCCCTGGTGTTTTTGGCGTCACCACTGGTGGCAATA |
| (without | | TGGGAGCTTAAGAAGGACGTCTACGTTGTCGAGCTGGATTGGTACCCAGACGCTC |
| donor | | CAGGAGAAATGGTCGTTCTGACGTGTGACACACCTGAGGAAGATGGTATTACCTG |
| template | | GACGCTTGATCAGTCATCCGAAGTTCTTGGGTCCGGGAAGACCCTTACAATCCAG |
| homology | | GTCAAAGAGTTCGGAGATGCTGGTCAGTATACTTGCCACAAGGGCGGTGAGGTCC |
| arms) | | TTAGTCACAGTTTGCTTCTGCTCCACAAGAAGGAGGACGGCATATGGAGCACAGA |
| | | TATATTGAAAGACCAAAAAGAACCCAAAAATAAGACATTCCTTCGCTGCGAGGCC |
| | | AAGAACTACAGCGGCCGGTTTACGTGCTGGTGGCTCACAACCATATCCACAGATC |
| | | TGACGTTCAGTGTTAAATCCTCAAGGGGTAGTAGCGATCCGCAAGGGGTTACGTG |
| | | CGGTGCTGCTACCCTTAGTGCTGAAAGGGTCAGAGGGGACAACAAAGAGTACGAA |
| | | TATAGTGTCGAATGCCAGGAAGATAGTGCGTGTCCGGCGGCAGAAGAGTCACTGC |
| | | CAATTGAGGTGATGGTCGACGCTGTGCACAAATTGAAATACGAGAATTATACCTC |
| | | AAGTTTCTTCATCAGAGATATTATAAAGCCTGACCCGCCCAAAAATTTGCAACTC |
| | | AAACCACTGAAAAATAGCCGCCAGGTGGAAGTCTCATGGGAATATCCTGATACCT |
| | | GGTCCACACCCCACTCCTATTTCTCACTCACATTTTGCGTTCAGGTCCAGGGAAA |
| | | GTCCAAGCGAGAAAAAAAAGATCGCGTTTTCACGGACAAAACCTCAGCCACAGTG |
| | | ATTTGCCGCAAGAATGCTTCCATATCCGTACGCGCTCAAGACAGGTATTACTCAT |
| | | CTTCATGGTCTGAATGGGCCTCTGTACCCTGTTCAGGAGGAGGTGGCAGTGGCGG |
| | | GGGCGGATCAGGCGGTGGAGGTAGCAGAAATTTGCCAGTGGCAACGCCAGATCCT |
| | | GGTATGTTCCCGTGCCTCCACCACTCTCAGAACCTCTTGAGGGCTGTGTCCAACA |
| | | TGTTGCAAAAGGCGCGCCAAACGCTCGAGTTTTACCCATGTACATCAGAGGAAAT |
| | | TGACCACGAGGACATTACGAAGGATAAAACCAGCACAGTAGAGGCATGTCTGCCA |
| | | TTGGAACTCACGAAAAACGAATCATGCCTTAACAGCCGAGAGACTTCTTTCATCA |
| | | CTAACGGATCTTGTCTTGCCTCAAGAAAGACTTCATTCATGATGGCCCTCTGCCT |
| | | CTCCTCAATCTACGAAGACCTCAAAATGTACCAAGTTGAGTTCAAGACCATGAAC |
| | | GCTAAACTCCTTATGGATCCAAAGCGCCAAATCTTTTTGGACCAAAACATGTTGG |
| | | CTGTGATAGACGAGCTGATGCAGGCTCTCAACTTCAATAGCGAGACCGTGCCCCA |
| | | AAAGTCATCCCTTGAAGAACCAGATTTTTATAAAACGAAGATTAAATTGTGTATT |
| | | CTGCTTCACGCTTTCCGGATCCGCGCTGTGACCATTGATCGAGTTATGTCTTATC |
| | | TGAACGCCTCTTAATAATAAAATCGCTATCCATCGAAGATGGATGTGTGTTGGTT |
| | | TTTTGTGTG |
| 3XAP-Min | 104 | ACGCGTGCTAGCCCGGGCACTGACTCATCAAGCACTGACTCATCAAGCACTGACT |
| IL2 | | CATCAAGGGACTCAGGGAGGGAAACTCCATTTTGACACCCCCATAATATTTTTCC |
| promoter | | AGAATTAACAGTATAAATTGCATCTCTTGTTCAAGAGTTCCCTATCACTCTCTTT |
| segment | | AATCACTACTCACAGTAACCTCAACTCCTGC |
| 5′ UTR | 105 | AAGGCCAGCCCAGCACCAGCACCAGCCAACTCTCACTGAAGCCAGCTCTCTCTTC |
| | | CTCCACCACC |

### Example 5: Inducible IL12 Knock-in Improves In Vivo Efficacy

This example shows efficacy of inducible (activation-dependent) IL12 knock-in into anti-CD70 CAR-T cells improvided in vivo anti-cancer efficacy in subcutaneous renal cell, non-small cell lung carcinoma, and pancreatic tumor xenogrograft mouse models.

The ability of T cells expressing a CD70 CAR and inducible IL12 construct to eliminate kidney carcinoma or non-small cell lung carcinoma cells expressing high levels of CD70 was evaluated in vivo using a subcutaneous renal cell carcinoma (A498 an CAKI-1), non-small cell lung carcinoma (NCI-H1975), and pancreatic cancer (BxCP3) xenografts model in mice.

CRISPR/Cas9 and AAV6 were used as above (see, e.g., Example 4) to create human T cells that lack expression of the TCR, β2M and CD70 with concomitant expression from the TRAC locus using a CAR construct targeting CD70 (SEQ ID NO: 19) and expression from the CD70 locus using an inducible IL12 construct targeting CD70. In this example activated T cells were first electroporated with Cas9:sgRNA RNP complexes containing sgRNAs targeting TRAC (SEQ ID NO: 27), β2M (SEQ ID NO: 41), and CD70 (SEQ ID NO: 59). The DNA double stranded break at the TRAC locus was repaired by homology directed repair with an AAV6-delivered DNA template comprising a donor template (SEQ ID NO: 21) (encoding anti-CD70 CAR comprising the amino acid sequence of SEQ ID NO: 19) containing right and left homology arms to the TRAC locus flanking a chimeric antigen receptor cassette (−/+ regulatory elements for gene expression). While the DNA double stranded break at the CD70 locus was repaired by homology directed repair with an AAV6-delivered DNA template comprising a donor template (SEQ ID NO: 99; CTX1569) (encoding inducible IL12 comprising the amino acid sequence of SEQ ID NO: 4) containing right and left homology arms to the CD70 locus. See also Example 4 above.

The resulting genetically modified T cells are:
3X KO (TRAC-/β2M-/CD70-/anti-CD70 CAR+ (with 41BB costimulatory domain)
3X KO (TRAC-/β2M-/CD70-/anti-CD70 CAR+(with 41BB costimulatory domain), plus inducible IL12 knock-in.

The ability of these anti-CD70 CAR+ T cells to ameliorate disease caused by a CD70+ renal carcinoma cell line or non-small cell lung cancer was evaluated in mouse model using methods employed by Translational Drug Development, LLC (Scottsdale, Ariz.). In brief, 5, 5-8 week old female, CIEA NOG (NOD.Cg-Prkdc^{scid}I12rg^{tm1Sug}ug/JicTac) for NCI-H1975 or NSG mice for CAKI-1 or A498, or NOG mice for BxCP3 cells were individually housed in ventilated microisolator cages, and maintained under pathogen-free conditions, 5-7 days prior to the start of the study. Mice received a subcutaneous inoculation of 5×10⁶NCI-H1975, CAM-1, A498, or BxCP3 cells/mouse in the right hind flank. When mean tumor size reached the required target tumor size of ˜100 mm³ for H1975 cells, ˜125 mm³ for CAKI-1 cells, ˜425 mm³ for A498 cells, or 100 mm³ for BxCP3 cells, the mice were further divided into one control group and two treatment groups as shown in Tables 8-10 below.

On Day 1, the treatment groups each received a single 200 ml intravenous dose of anti-CD70 CAR+ T cells with without inducible IL12 as shown in Tables 8-10.

For NOG mice inoculated with NCI-H1975, 1×10⁷ CAR-T cells were administered to each mouse. For NSG mice inoculated with CAKI-1 or A498 cells, 8×10⁶CAR-T cells were administered to each mouse. For NOG mice inoculated with BxCP3, 1×10⁷ cells were administered to each mouse.

TABLE 8
Treatment groups (NCI-H1975)

| Group | CAR-T | T cell treatment (i.v.) | N |
|---|---|---|---|
| 1 | None | None | 5 |
| 2 | CD70 CART | 1 × 10⁷ cells/mouse | 5 |
| 3 | CD70 CART+ Inducible I112 | 1 × 10⁷ cells/mouse | 5 |

TABLE 9
Treatment groups (CAKI-1 or A498)

| Group | CAR-T | T cell treatment (i.v.) | N |
|---|---|---|---|
| 1 | None | None | 5 |
| 2 | CD70 CART | 8 × 10⁶ cells/mouse | 5 |
| 3 | CD70 CART+ Inducible I112 | 8 × 10⁶ cells/mouse | 5 |

TABLE 10
Treatment groups (BxCP3)

| Group | CAR-T | T cell treatment (i.v.) | N |
|---|---|---|---|
| 1 | None | None | 5 |
| 2 | CD70 CART | 1 × 10⁷ cells/mouse | 5 |
| 3 | CD70 CART+ Inducible I112 | 1 × 10⁷ cells/mouse | 5 |

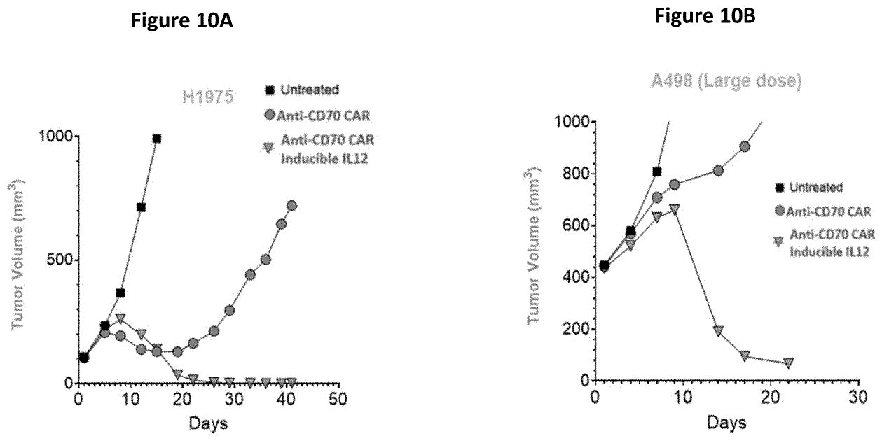

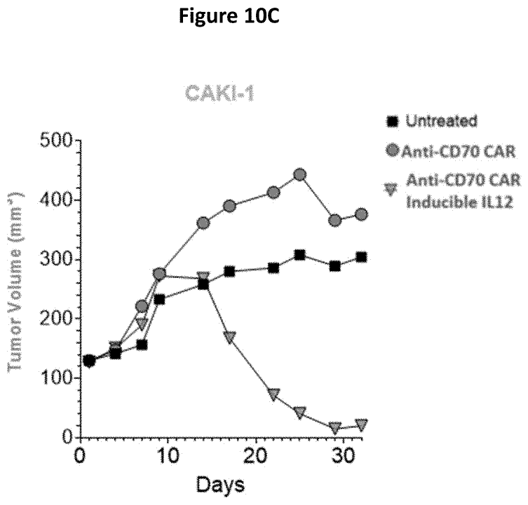

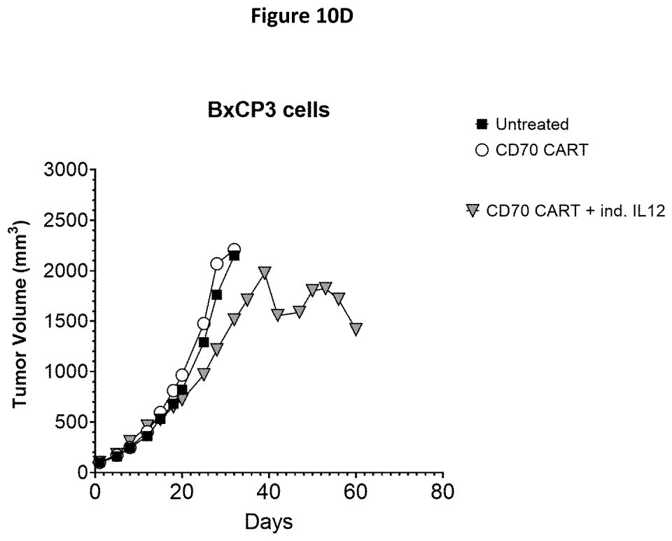

Tumor volume was measured 2 times weekly from day of treatment initiation. As shown in FIGS. 10A-10C, inducible IL12 expression improves the potency of anti-CD70 CAR T cells in vivo. Anti-CD70 CART without an IL 12 knock-in showed some control over the NCI-H1975 tumors but was unable to produce complete tumor regression (FIG. 10A). Anti-CD70 CAR T cells that also expressed IL12 were able to cause complete regression of non-small cell lung carcinoma cells (NCI-H1975) (FIG. 10A). In the RCC studies using A498 and CAM-1 tumor cells, the addition of an inducible IL12 construct also increased efficacy of the anti-CD70 CAR T cells compared to cells without IL12. FIGS. 10B and 10C. The anti-CD70 CAR T cells with inducible IL12 were able to produce complete regression of renal cell carcinoma in vivo.

In the Pancreatic tumor model using BxCP3 tumor cells, the addition of an inducible IL12 construct also increased efficacy of the anti-CD70 CAR T cells compared to cells without IL12 (FIG. 10D). The anti-CD70 CAR T cells with inducible IL12 were able to slow tumor progression of pancreatic cancer cells in vivo.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A population of genetically engineered immune cells, comprising immune cells that express an interleukin 12 (IL12) protein upon activation of the immune cells, wherein the immune cells comprise an expression cassette of the IL12 protein, the expression cassette comprising a transgene encoding the IL12 protein, a promoter operably linked to the transgene, and a binding site of a transcriptional regulatory factor associated with immune cell activation.

2. The population of genetically engineered immune cells of claim 1, wherein the binding site comprises an NFκb binding site, an AP-1 binding site, a STAT5 binding site, a SMAD binding site, an NFAT binding site, or a combination thereof.

3. The population of genetically engineered immune cells of claim 2, wherein the binding site comprises multiple copies of a binding motif of NFκb, AP-1, STATS, SMAD, and/or NFAT.

4. The population of genetically engineered immune cells of claim 1, wherein the promoter is an IL2 promoter or a late ADE promoter.

5. The population of genetically engineered immune cells of claim 1, wherein the immune cells comprise a nucleic acid encoding a chimeric antigen receptor (CAR) specific to an antigen of interest.

6. The population of genetically engineered immune cells of claim 5, wherein the immune cells further comprise a disrupted T cell receptor alpha chain constant (TRAC) gene, a disrupted beta-2-microglubulin (β2M) gene, a disrupted gene encoding the antigen of interest, or a combination thereof.

7. The population of genetically engineered immune cells of claim 5, wherein the nucleic acid encoding the CAR is inserted into a first genomic locus.

8. The population of genetically engineered immune cells of claim 7, wherein the nucleic acid encoding the CAR is inserted into the first genomic locus by CRISPR/Cas-mediated gene editing and homologous recombination.

9. The population of genetically engineered immune cells of claim 8, wherein the CRISPR/Cas-mediated gene editing involves a first guide RNA targeting a site in the first genomic locus, and wherein the nucleic acid encoding the CAR is inserted at the site in the first genomic locus.

10. The population of genetically engineered immune cells of claim 7, wherein the first genomic locus is the TRAC gene and insertion of the nucleic acid encoding the CAR disrupts expression of the TRAC gene.

11. The population of genetically engineered immune cells of claim 9, wherein the first guide RNA targets a TRAC site comprising the nucleotide sequence of 5′-AGAGCAACAGTGCTGTGGCC-3′ (SEQ ID NO: 22).

12. The population of genetically engineered immune cells of claim 11, wherein the nucleic acid encoding the CAR is inserted at the TRAC site comprising the nucleotide sequence of 5′-AGAGCAACAGTGCTGTGGCC-3′ (SEQ ID NO: 22).

13. The population of genetically engineered immune cells of claim 12, wherein the disrupted TRAC gene has a deletion of a fragment comprising 5′-AGAGCAACAGTGCTGTGGCC-3′ (SEQ ID NO: 22), which is replaced by the nucleic acid encoding the CAR.

14. The population of genetically engineered immune cells of claim 1, wherein the expression cassette of the IL12 protein is inserted into a second genomic locus.

15. The population of genetically engineered immune cells of claim 14, wherein the expression cassette of the IL12 protein is inserted into the second genomic locus by CRISPR/Cas-mediated gene editing and homologous recombination.

16. The population of genetically engineered immune cells of claim 15, wherein CRISPR/Cas-mediated gene editing involves a second guide RNA targeting a site in the second genomic locus, and wherein the expression cassette of the IL12 protein is inserted at site in the second genomic locus.

17. The population of genetically engineered immune cells of claim 14, wherein the second target genomic locus is AAVS1, 132M, or the gene encoding the antigen of interest.

18. The population of genetically engineered immune cell of claim 17, wherein the second genomic locus is AAVS1 and the second guide RNA targets an AAVS1 site comprising the nucleotide sequence of 5′-GGGGCCACTAGGGACAGGAT-3′ (SEQ ID NO: 48).

19. The population of genetically engineered immune cell of claim 18, wherein the expression cassette of the IL12 protein is inserted into the AAVS1 site comprising the nucleotide sequence of 5′-GGGGCCACTAGGGACAGGAT-3′ (SEQ ID NO: 48).

20. The population of genetically engineered immune cell of claim 19, wherein the expression cassette of the IL12 protein replaces a fragment comprising the nucleotide sequence of 5′-GGGGCCACTAGGGACAGGAT-3′ (SEQ ID NO: 48) in the AAVS1 genomic locus.

21. The population of genetically engineered immune cells of claim 17, wherein the second genomic locus is β2M and the second guide RNA targets a β2M site comprising the nucleotide sequence of 5′-GCTACTCTCTCTTTCTGGCC-3′ (SEQ ID NO: 36).

22. The population of genetically engineered immune cells of claim 21, wherein the expression cassette of the IL12 protein is inserted into the β2M site comprising the nucleotide sequence of 5′-GCTACTCTCTCTTTCTGGCC-3′ (SEQ ID NO: 36).

23. The population of genetically engineered immune cells of claim 22, wherein the expression cassette of the IL12 protein replaces a fragment comprising the nucleotide sequence of 5′-GCTACTCTCTCTTTCTGGCC-3′ (SEQ ID NO: 36) in the β2M gene.

24. The population of genetically engineered immune cells of claim 5, wherein the antigen of interest is a tumor-associated antigen.

25. The population of genetically engineered immune cells of claim 24, wherein the antigen of interest is CD70.

26. The population of genetically engineered immune cells of claim 5, wherein the antigen of interest is CD70, and wherein the expression cassette of the IL12 is inserted into the CD70 gene locus, thereby disrupting expression of the CD70 gene.

27. The population of genetically engineered immune cells of claim 26, wherein the second guide RNA targets a CD70 site comprising the nucleotide sequence of 5′-GCTTTGGTCCCATTGGTCGC-3′ (SEQ ID NO: 54).

28. The population of genetically engineered immune cells of claim 27, wherein the expression cassette of the IL12 protein is inserted in the CD70 site comprising the nucleotide sequence of 5′-GCTTTGGTCCCATTGGTCGC-3′ (SEQ ID NO: 54).

29. The population of genetically engineered immune cells of claim 28, wherein the expression cassette of the IL12 protein replaces a fragment comprising the nucleotide sequence of 5′-GCTTTGGTCCCATTGGTCGC-3′ (SEQ ID NO: 54) in the CD70 gene.

30. The population of genetically engineered immune cells of claim 24, wherein the CAR binds the tumor-associated antigen.

31. The population of genetically engineered immune cells of claim 25, wherein the CAR binds CD70 and comprises an extracellular domain, a CD8 transmembrane domain, a 4-1BB co-stimulatory domain or a CD28 co-stimulatory domain, and a CD3ζ cytoplasmic signaling domain, and wherein the extracellular domain is a single-chain antibody fragment (scFv) that binds CD70; optionally wherein the CAR that binds CD70 comprises the 4-1BB co-stimulatory domain.

32. The population of genetically engineered immune cells of claim 31, wherein the scFv comprises a heavy chain variable domain (V_{H}) comprising SEQ ID NO: 9, and a light chain variable domain (V_{L}) comprising SEQ ID NO: 10.

33. The population of genetically engineered immune cells of claim 32, wherein the scFv comprises SEQ ID NO: 8.

34. The population of the genetically engineered immune cells of claim 33, wherein the CAR comprises SEQ ID NO: 19.

35. The population of genetically engineered immune cells of claim 1, wherein the immune cells are human T cells.

36. The population of genetically engineered immune cells of claim 1, wherein the binding site in the expression cassette of the IL12 protein comprises one or more AP-1 binding sites, optionally wherein the binding site comprises three AP-1 binding sites.

37. The population of genetically engineered immune cells of claim 36, wherein the AP-1 binding site comprises the nucleotide sequence of SEQ ID NO:67.

38. The population of genetically engineered immune cells of claim 36, wherein the promoter in the expression cassette of the IL12 protein comprises a minimal IL2 promoter.

39. The population of genetically engineered immune cells of claim 38, wherein the minimal IL2 promoter comprises the nucleotide sequence of SEQ ID NO:70.

40. The population of genetically engineered immune cells of claim 1, wherein the IL12 protein is a single chain polypeptide comprising IL12p40 and IL12p35.

41. The population of genetically engineered immune cells of claim 40, wherein the IL12 protein comprises the amino acid sequence of SEQ ID NO: 3 or SEQ ID NO: 4.

42. A population of genetically engineered immune cells, comprising immune cells that, collectively, comprise an expression cassette of an interleukin 12 (IL12) protein, and a nucleic acid encoding a chimeric antigen receptor (CAR) specific to an antigen of interest.

43. The population of genetically engineered immune cells of claim 42, further comprising a disrupted TRAC gene, a disrupted β2M gene, a disrupted gene encoding the antigen of interest, or a combination thereof.

44. The population of genetically engineered immune cells of claim 43, wherein at least a portion of the immune cells each comprise an expression cassette of an interleukin 12 (IL12) protein, a nucleic acid encoding a chimeric antigen receptor (CAR) specific to an antigen of interest, a disrupted TRAC gene, and a disrupted β2M gene.

45. The population of genetically engineered immune cells of claim 43, wherein at least a portion of the immune cells each comprise an expression cassette of an interleukin 12 (IL12) protein, a nucleic acid encoding a chimeric antigen receptor (CAR) specific to an antigen of interest, and a disrupted gene encoding the antigen of interest.

46. The population of genetically engineered immune cells of claim 43, wherein at least a portion of the immune cells each comprise an expression cassette of an interleukin 12 (IL12) protein, a nucleic acid encoding a chimeric antigen receptor (CAR) specific to an antigen of interest, a disrupted TRAC gene, a disrupted β2M gene, and a disrupted gene encoding the antigen of interest.

47. The population of genetically engineered immune cells of claim 42, wherein the nucleic acid encoding the CAR is inserted into a first genomic locus.

48. The population of genetically engineered immune cells of claim 47, wherein the nucleic acid encoding the CAR is inserted into the first genomic locus by CRISPR/Cas-mediated gene editing and homologous recombination.

49. The population of genetically engineered immune cells of claim 48, wherein the CRISPR/Cas-mediated gene editing involves a first guide RNA targeting a site in the first genomic locus, and wherein the nucleic acid is inserted at the site in the first genomic locus.

50. The population of genetically engineered immune cells of claim 47, wherein the first genomic locus is the TRAC gene and insertion of the nucleic acid encoding the CAR disrupts expression of the TRAC gene.

51. The population of genetically engineered immune cells of claim 50, wherein the first guide RNA targets a TRAC site comprising the nucleotide sequence of 5′-AGAGCAACAGTGCTGTGGCC-3′ (SEQ ID NO: 22).

52. The population of genetically engineered immune cells of claim 51, wherein the nucleic acid encoding the CAR is inserted at the TRAC site comprising the nucleotide sequence of 5′-AGAGCAACAGTGCTGTGGCC-3′ (SEQ ID NO: 22).

53. The population of genetically engineered immune cells of claim 52, wherein the disrupted TRAC gene has a deletion of a fragment comprising 5′-AGAGCAACAGTGCTGTGGCC-3′ (SEQ ID NO: 22), which is replaced by the nucleic acid encoding the CAR.

54. The population of genetically engineered immune cells of claim 42, wherein the expression cassette of the IL12 protein is inserted into a second genomic locus.

55. The population of genetically engineered immune cells of claim 54, wherein the expression cassette of the IL12 protein is inserted into the second genomic locus by CRISPR/Cas-mediated gene editing and homologous recombination.

56. The population of genetically engineered immune cells of claim 50, wherein CRISPR/Cas-mediated gene editing involves a second guide RNA targeting a site in the second genomic locus, and wherein the expression cassette of the IL12 protein is inserted at site in the second genomic locus.

57. The population of genetically engineered immune cells of claim 54, wherein the second target genomic locus is AAVS1, β2M, or the gene encoding the antigen of interest.

58. The population of genetically engineered immune cell of claim 57, wherein the second genomic locus is AAVS1 and the second guide RNA targets an AAVS1 site comprising the nucleotide sequence of 5′-GGGGCCACTAGGGACAGGAT-3′ (SEQ ID NO: 48).

59. The population of genetically engineered immune cell of claim 58, wherein the expression cassette of the IL12 protein is inserted in the AAVS1 site comprising the nucleotide sequence of 5′-GGGGCCACTAGGGACAGGAT-3′ (SEQ ID NO: 48).

60. The population of genetically engineered immune cell of claim 59, wherein the expression cassette of the IL12 protein replaces a fragment comprising the nucleotide sequence of 5′-GGGGCCACTAGGGACAGGAT-3′ (SEQ ID NO: 48) in the AAVS1 genomic locus.

61. The population of genetically engineered immune cells of claim 60, wherein first genomic locus is β2M and the second guide RNA targets a β2M site comprising the nucleotide sequence of 5′-GCTACTCTCTCTTTCTGGCC-3′ (SEQ ID NO: 36).

62. The population of genetically engineered immune cells of claim 61, wherein the expression cassette of the IL12 protein is inserted in the β2M site comprising the nucleotide sequence of 5′-GCTACTCTCTCTTTCTGGCC-3′ (SEQ ID NO: 36).

63. The population of genetically engineered immune cells of claim 62, wherein the expression cassette of the IL12 protein replaces a fragment comprising the nucleotide sequence of 5′-GCTACTCTCTCTTTCTGGCC-3′ (SEQ ID NO: 36) in the β2M gene.

64. The population of genetically engineered immune cells of claim 42, wherein the antigen of interest is a tumor-associated antigen.

65. The population of genetically engineered immune cells of claim 64, wherein the antigen of interest is CD70.

66. The population of genetically engineered immune cells of claim 42, wherein the antigen of interest is CD70, and wherein the expression cassette of the IL12 is inserted in the CD70 gene locus, thereby disrupting expression of the CD70 gene.

67. The population of genetically engineered immune cells of claim 66, wherein the second guide RNA targets a CD70 site comprising the nucleotide sequence of 5′-GCTTTGGTCCCATTGGTCGC-3′ (SEQ ID NO: 54).

68. The population of genetically engineered immune cells of claim 67, wherein the expression cassette of the IL12 protein is inserted in the CD70 site comprising the nucleotide sequence of 5′-GCTTTGGTCCCATTGGTCGC-3′ (SEQ ID NO: 54).

69. The population of genetically engineered immune cells of claim 68, wherein the expression cassette of the IL12 protein replaces a fragment comprising the nucleotide sequence of 5′-GCTTTGGTCCCATTGGTCGC-3′ (SEQ ID NO: 54) in the CD70 gene.

70. The population of genetically engineered immune cells of claim 42, wherein the CAR binds the tumor-associated antigen.

71. The population of genetically engineered immune cells of claim 70, wherein the CAR binds CD70 and comprises an extracellular domain, a CD8 transmembrane domain, a 4-1BB co-stimulatory domain or a CD28 co-stimulatory domain, and a CD3ζ cytoplasmic signaling domain, and wherein the extracellular domain is a single-chain antibody fragment (scFv) that binds CD70; optionally wherein the CAR that binds CD70 comprises the 4-1BB co-stimulatory domain.

72. The population of genetically engineered immune cells of claim 71, wherein the scFv comprises a heavy chain variable domain (V_{H}) comprising SEQ ID NO: 9, and a light chain variable domain (V_{L}) comprising SEQ ID NO: 10.

73. The population of genetically engineered immune cells of claim 72, wherein the scFv comprises SEQ ID NO: 8.

74. The population of the genetically engineered immune cells of claim 73, wherein the CAR comprises SEQ ID NO: 19.

75. The population of genetically engineered immune cells of claim 42, wherein the immune cells are human T cells.

76. The population of genetically engineered immune cells of claim 42, wherein the binding site in the expression cassette of the IL12 protein comprises one or more AP-1 binding sites, optionally wherein the binding site comprises three AP-1 binding site.

77. The population of genetically engineered immune cells of claim 76, wherein the AP-1 binding site comprises the nucleotide sequence of SEQ ID NO:67.

78. The population of genetically engineered immune cells of claim 42, wherein the promoter in the expression cassette of the IL12 protein comprises an IL2 promoter or an ADE promoter.

79. The population of genetically engineered immune cells of claim 78, wherein the promoter in the expression cassette of the IL12 protein is a minimal IL2 promoter.

80. The population of genetically engineered immune cells of claim 79, wherein the minimal IL2 promoter comprises the nucleotide sequence of SEQ ID NO:70.

81. The population of genetically engineered immune cells of claim 42, wherein the IL12 protein is a single chain polypeptide comprising IL12p40 and IL12p35.

82. The population of genetically engineered immune cells of claim 81, wherein the IL12 protein comprising the amino acid sequence of SEQ ID NO: 3 or SEQ ID NO: 4.

83. A method for producing genetically engineered immune cells, the method comprising:
(i) introducing into a population of immune cells an expression cassette of an interleukin 12 (IL12) protein, wherein the expression cassette comprising a transgene encoding the IL12 protein, a promoter in operably linkage to the transgene, and a binding site of a transcriptional regulatory factor associated with immune cell activation; and
(ii) harvesting the genetically engineered immune cells produced in step (i).

84. The method of claim 83, wherein the binding site comprises an NFκb binding site, an AP-1 binding site, a STATS binding site, a SMAD binding site, an NFAT binding site, or a combination thereof.

85. The method of claim 84, wherein the binding site comprises multiple copies of a binding motif of NFκb, AP-1, STATS, SMAD, and/or NFAT.

86. The method of claim 83, wherein step (i) is performed by delivering to the population of immune cells:
(a) a first RNA-guided nuclease,
(b) a first guide RNA (gRNA) targeting a genomic locus of interest; and
(c) a first vector comprising (1) the expression cassette of the IL12 protein, and (2) a first upstream and a first downstream nucleotide sequences flanking the expression cassette, wherein the first upstream nucleotide sequence comprises a left region of homology to the first genomic locus of interest and the first downstream nucleotide sequence comprises a right region of homology to the first genomic locus of interest.

87. The method of claim 83, wherein in step (i), the population of immune cells comprise a nucleic acid encoding a chimeric antigen receptor (CAR) specific to an antigen of interest.

88. The method of claim 83, wherein in step (i), the population of immune cells comprise, collectively, (1) a nucleic acid encoding a chimeric antigen receptor (CAR) specific to an antigen of interest, and (2) a disrupted TRAC gene, a disrupted β2M gene, and disrupted gene encoding the antigen of interest, or a combination thereof.

89. The method of claim 88, wherein the nucleic acid encoding the CAR is inserted into the TRAC gene, thereby disrupting expression of the TRAC gene.

90. The method of claim 89, wherein the nucleic acid encoding the CAR is inserted in a TRAC gene site comprising the nucleotide sequence of 5′-AGAGCAACAGTGCTGTGGCC-3′ (SEQ ID NO: 22).

91. The method of claim 88, wherein the disrupted TRAC gene has a deletion of the nucleotide sequence of 5′-AGAGCAACAGTGCTGTGGCC-3′ (SEQ ID NO: 22).

92. The method of claim 91, wherein the nucleic acid encoding the CAR replaces a fragment comprising the nucleotide sequence of 5′-AGAGCAACAGTGCTGTGGCC-3′ (SEQ ID NO: 22).

93. The method of claim 83, further comprising delivering to the immune cells:
(a) a second RNA-guided nuclease,
(b) a second guide RNA (gRNA) targeting a TRAC gene locus, and
(c) a second vector comprising (1) a nucleic acid encoding a chimeric antigen receptor (CAR) specific to an antigen of interest, and (2) a second upstream and a second downstream nucleotide sequences flanking the nucleic acid encoding the CAR, wherein the second upstream nucleotide sequence comprises a left region homology to the TRAC gene locus and the second downstream nucleotide sequence comprises a right region homology to the TRAC gene locus.

94. The method of claim 93, wherein the second gRNA targeting a TRAC site comprising the nucleotide sequence of 5′-AGAGCAACAGUGCUGUGGCC-3′ (SEQ ID NO: 25).

95. The method of claim 83, further comprising delivering to the immune cells (g) a third guide RNA (gRNA) targeting a β2M gene locus, a fourth guide RNA (gRNA) targeting a gene encoding the antigen of interest, or a combination thereof.

96. The method of claim 86, wherein the genomic locus of interest is AAVS1, β2M, or a gene encoding the antigen of interest.

97. The method of claim 96, wherein the genomic locus of interest first is β2M.

98. The method of claim 97, wherein the third gRNA targeting a β2M site comprising the nucleotide sequence of 5′-GCUACUCUCUCUUUCUGGCC-3′ (SEQ ID NO: 38).

99. The method of claim 98, wherein the first gRNA and the third gRNA are the same gRNA.

100. The method of claim 87, wherein the antigen of interest is a tumor-associated antigen.

101. The method of claim 96, wherein the antigen of interest is CD70.

102. The method of claim 100, wherein the antigen of interest is CD70 and the fourth gRNA targets a CD70 gene site comprising the nucleotide sequence of 5′-GCUUUGGUCCCAUUGGUCGC-3′ (SEQ ID NO: 56).

103. The method of claim 102, wherein the first gRNA and the fourth gRNA are the same gRNA.

104. The method of claim 96, wherein the genomic locus of interest is AAVS1.

105. The method of claim 104, wherein the first guide RNA targets an AAVS1 site comprising the nucleotide sequence of 5′-GGGGCCACTAGGGACAGGAT-3′ (SEQ ID NO: 48).

106. The method of claim 87, wherein the CAR binds a tumor-associated antigen.

107. The method of claim 87, wherein the CAR binds CD70 and comprises an extracellular domain, a CD8 transmembrane domain, a 4-1BB co-stimulatory domain or a CD-28 co-stimulary domain, and a CD3ζ cytoplasmic signaling domain, and wherein the extracellular domain is a single-chain antibody fragment (scFv) that binds CD70; optionally wherein the CAR that binds CD70 comprises the 4-1BB co-stimulatory domain.

108. The method of claim 107, wherein the scFv comprises a heavy chain variable domain (V_{H}) comprising SEQ ID NO: 9, and a light chain variable domain (V_{L}) comprising SEQ ID NO: 10.

109. The method of claim 108, wherein the scFv comprises SEQ ID NO: 8.

110. The method of claim 109, wherein the CAR comprises SEQ ID NO: 19.

111. The method of claim 83, wherein the immune cells are human T cells.

112. The method of claim 83, wherein the binding site in the expression cassette of the IL12 protein comprises one or more AP-1 binding sites, optionally wherein the binding site comprises three AP-1 binding site.

113. The method of claim 112, wherein the AP-1 binding site comprises the nucleotide sequence of SEQ ID NO:67.

114. The method of claim 83, wherein the promoter in the expression cassette of the IL12 protein comprises an IL2 promoter or an ADE promoter.

115. The method of claim 114, wherein the promoter is the IL2 promoter, which is a minimal IL2 promoter, optionally wherein the minimal IL2 promoter comprises the nucleotide sequence of SEQ ID NO:70.

116. The method of claim 83, wherein the IL12 protein is a single chain polypeptide comprising IL12p40 and IL12p35.

117. The method of claim 116, wherein the IL12 protein comprising the amino acid sequence of SEQ ID NO: 3 or SEQ ID NO: 4.

118. The method of claim 86, wherein the first vector, the second vector, or both are AAV vectors.

119. The method of claim 86, wherein the first RNA-guided nuclease, the second RNA-guided nuclease, or both are a Cas9 enzyme.

120. The method of claim 86, wherein the first RNA-guide nuclease and the second RNA-guided nuclease are the same enzyme.

121. A method for producing chimeric antigen receptor (CAR) T cells secreting an interleukin 12 protein, the method comprising:
(i) delivering to a population of immune cells
(a) a first vector comprising a nucleic acid encoding an interleukin 12 protein, and
(b) a second vector comprising a nucleic acid encoding a chimeric antigen receptor (CAR) specific to an antigen of interest; and
(ii) harvesting genetically engineered immune cells produced in step (i) expressing the CAR and the IL12 protein.

122. The method of claim 121, further comprising delivering to the population of immune cells:
(c) one or more RNA-guided nucleases, and
(d) a first guide RNA (gRNA) targeting a TRAC gene locus, a second gRNA targeting a β2M gene locus, a third gRNA targeting a genomic locus of interest, a fourth gRNA targeting a gene encoding the antigen of interest, or a combination thereof.

123. The method of claim 122, wherein the first vector further comprises a first upstream and a first downstream nucleotide sequences flanking the expression cassette, and wherein the first upstream nucleotide sequence comprises a left region of homology to the genomic locus of interest and the first downstream nucleotide sequence comprises a right region of homology to the genomic locus of interest.

124. The method of claim 123, wherein the genomic locus of interest is AAVS1, β2M, or a gene encoding the antigen of interest.

125. The method of claim 124, wherein the genomic locus of interest is β2M.

126. The method of claim 125, wherein the second gRNA targeting a β2M site comprising the nucleotide sequence of 5′-GCUACUCUCUCUUUCUGGCC-3′ (SEQ ID NO: 38).

127. The method of claim 126, wherein the second gRNA and the third gRNA are the same gRNA.

128. The method of claim 121, wherein the antigen of interest is a tumor-associated antigen.

129. The method of claim 128, wherein the antigen of interest is CD70.

130. The method of claim 129, wherein the genomic locus of interest is the gene encoding the antigen of interest, which is CD70, and wherein the fourth gRNA targets a CD70 gene site comprising the nucleotide sequence of 5′-GCUUUGGUCCCAUUGGUCGC-3′ (SEQ ID NO: 56).

131. The method of claim 130, wherein the third gRNA and the fourth gRNA are the same gRNA.

132. The method of claim 124, wherein the genomic locus of interest is AAVS1.

133. The method of claim 132, wherein the third guide RNA targets an AAVS1 site comprising the nucleotide sequence of 5′-GGGGCCACTAGGGACAGGAT-3′ (SEQ ID NO: 48).

134. The method of claim 123, wherein the second vector further comprises a second upstream and a second downstream nucleotide sequences flanking the nucleic acid encoding the CAR, and wherein the second upstream nucleotide sequence comprises a left region homology to the TRAC gene locus and the second downstream nucleotide sequence comprises a right region homology to the TRAC gene locus.

135. The method of claim 134, wherein the first gRNA targets a TRAC site comprising the nucleotide sequence of 5′-AGAGCAACAGUGCUGUGGCC-3′ (SEQ ID NO: 25).

136. The method of claim 121, wherein step (i) is performed by delivering components (a)-(d) simultaneously.

137. The method of claim 136, wherein step (i) is performed by delivering the first vector, the second vector, and a ribonucleoprotein (RNP) comprising the RNA-guided nuclease(s) and the gRNAs via one electroporation.

138. The method of claim 121, wherein step (i) is performed by delivering components (a)-(d) sequentially, optionally via multiple electroporation.

139. The method of claim 121, wherein the CAR binds CD70 and comprises an extracellular domain, a CD8 transmembrane domain, a 4-1BB co-stimulatory domain or a CD28 co-stimulatory domain, and a CD3ζ cytoplasmic signaling domain, and wherein the extracellular domain is a single-chain antibody fragment (scFv) that binds CD70; optionally wherein the CAR that binds CD70 comprises the 4-1BB co-stimulatory domain.

140. The method of claim 139, wherein the scFv comprises a heavy chain variable domain (V_{H}) comprising SEQ ID NO: 9, and a light chain variable domain (V_{L}) comprising SEQ ID NO: 10.

141. The method of claim 140, wherein the scFv comprises SEQ ID NO: 8.

142. The method of claim 141, wherein the CAR comprises SEQ ID NO: 19.

143. The method of claim 121, wherein the immune cells are human T cells.

144. The method of claim 121, wherein the binding site in the expression cassette of the IL12 protein comprises one or more AP-1 binding sites, optionally wherein the binding site comprises three AP-1 binding site.

145. The method of claim 144, wherein the AP-1 binding site comprises the nucleotide sequence of SEQ ID NO:67.

146. The method of claim 121, wherein the promoter in the expression cassette of the IL12 protein comprises an IL2 promoter or an ADE promoter.

147. The method of claim 146, wherein the promoter is the IL2 promoter, which is a minimal IL2 promoter, optionally wherein the minimal IL2 promoter comprises the nucleotide sequence of SEQ ID NO:70.

148. The method of claim 121, wherein the IL12 protein is a single chain polypeptide comprising IL12p40 and IL12p35.

149. The method of claim 148, wherein the IL12 protein comprising the amino acid sequence of SEQ ID NO: 3 or SEQ ID NO: 4.

150. The method of claim 121, wherein the first vector comprises an expression cassette, which comprises the nucleic acid encoding the IL12 protein, a promoter in operable linkage to the nucleic acid, and a binding site of a transcriptional regulatory factor associated with immune cell activation.

151. The method of claim 150, wherein the binding site comprises an NFκb binding site, an AP-1 binding site, a STATS binding site, a SMAD binding site, an NFAT binding site, or a combination thereof.

152. The method of claim 151, wherein the binding site comprises multiple copies of a binding motif of NFκb, AP-1, STATS, SMAD, and/or NFAT.

153. The method of claim 121, wherein the first vector, the second vector, or both are AAV vectors.

154. The method of claim 121, wherein the one or more RNA-guided nuclease are a Cas9 enzyme.

155. A genetically engineered immune cells, which is prepared by a method of claim 83.

156. A method for treating a solid tumor or a hematopoietic maligancy, the method comprising administering to a subject in need thereof an effective amount of a population of immune cells set forth in claim 1.

157. The method of claim 156, wherein the population of immune cells is allogeneic.

158. The method of claim 156, wherein the subject is a human patient having a solid tumor or a hematopoietic maligancy.

159. The method of claim 158, wherein the human patient has a solid tumor, which is renal cell carcinoma, lung cancer, or pancreatic cancer, optionally wherein the lung cancer is non-small cell lung cancer.

160. The method of claim 158, wherein the human patient has hematopoitic malignancy.

161. The method of claim 160, wherein the hematopoietic malignancy is a T cell malignancy or a B cell malignancy.

162. The method of claim 161, wherein the T or B cell malignancy is selected from the group consisting of peripheral T cell lymphoma (PTCL), anaplastic large cell lymphoma (ALCL), Sezary syndrome (SS), non-smoldering acute adult T cell leukemia or lymphoma (ATLL), angioimmunoblastic T cell lymphoma (AITL), and diffuse large B cell lymphoma (DLBCL).

163. The method of claim 156, wherein the subject has undergone a lymphodepletion treatment prior to administration of the population of immune cells.

164. The method of claim 156, further comprising subjecting the subject to a lymphodepleting treatment prior to administration of the population of immune cells.


SEQUENCE LISTING (105 sequences)

SEQ ID NO: 1 - protein, 196 aa, Artificial Sequence
  Synthetic
NLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 306 aa, Artificial Sequence
  Synthetic
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 540 aa, Artificial Sequence
  Synthetic
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 540 aa, Artificial Sequence
  Synthetic
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 23 aa, Artificial Sequence
  Synthetic
MCPARSLLLVATLVLLDHLSLAR

SEQ ID NO: 6 - protein, 22 aa, Artificial Sequence
  Synthetic
MCHQQLVISWFSLVFLASPLVA

SEQ ID NO: 7 - protein, 15 aa, Artificial Sequence
  Synthetic
GGGGSGGGGSGGGGS

SEQ ID NO: 8 - protein, 245 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 9 - protein, 118 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 10 - protein, 111 aa, Artificial Sequence
  Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 11 - protein, 16 aa, Artificial Sequence
  Synthetic
GGGGSGGGGSGGGGSG

SEQ ID NO: 12 - protein, 22 aa, Artificial Sequence
  Synthetic
MLLLVTSLLLCELPHPAFLLIP

SEQ ID NO: 13 - protein, 21 aa, Artificial Sequence
  Synthetic
MALPVTALLLPLALLLHAARP

SEQ ID NO: 14 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 15 - protein, 23 aa, Artificial Sequence
  Synthetic
IYIWAPLAGTCGVLLLSLVITLY

SEQ ID NO: 16 - protein, 42 aa, Artificial Sequence
  Synthetic
KRGRKKLLYIFKQPFMRPVQTTQEEDGCSCRFPEEEEGGCEL

SEQ ID NO: 17 - protein, 40 aa, Artificial Sequence
  Synthetic
SKRSRLLHSDYMNMTPRRPGPTRKHYQPYAPPRDFAAYRS

SEQ ID NO: 18 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 508 aa, Artificial Sequence
  Synthetic
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 21 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 22 - DNA, 20 bp, Artificial Sequence
  Synthetic
agagcaacagtgctgtggcc

SEQ ID NO: 23 - DNA, 23 bp, Artificial Sequence
  Synthetic
agagcaacagtgctgtggcctgg

SEQ ID NO: 24 - RNA, 100 nt, Artificial Sequence
  Synthetic
agagcaacagugcuguggccguuuuagagcuagaaauagcaaguuaaaauaaggcuaguccguuaucaacuugaaaaaguggcaccgagucggugcuuuu

SEQ ID NO: 25 - RNA, 20 nt, Artificial Sequence
  Synthetic
agagcaacagugcuguggcc

SEQ ID NO: 26 - RNA, 20 nt, Artificial Sequence
  Synthetic
  misc_feature (1)..(4): modified with 2′-O-methyl phosphorothioate
agagcaacagugcuguggcc

SEQ ID NO: 27 - RNA, 100 nt, Artificial Sequence; identical to SEQ ID NO: 24
  Synthetic
  misc_feature (1)..(4): modified with 2′-O-methyl phosphorothioate
  misc_feature (97)..(100): modified with 2′-O-methyl phosphorothioate

SEQ ID NO: 28 - DNA, 19 bp, Artificial Sequence
  Synthetic
aagagcaacaaatctgact

SEQ ID NO: 29 - DNA, 58 bp, Artificial Sequence
  Synthetic
aagagcaacagtgctgtgcctggagcaacaaatctgactaagagcaacaaatctgact

SEQ ID NO: 30 - DNA, 52 bp, Artificial Sequence
  Synthetic
aagagcaacagtgctggagcaacaaatctgactaagagcaacaaatctgact

SEQ ID NO: 31 - DNA, 53 bp, Artificial Sequence
  Synthetic
aagagcaacagtgcctggagcaacaaatctgactaagagcaacaaatctgact

SEQ ID NO: 32 - DNA, 38 bp, Artificial Sequence
  Synthetic
aagagcaacagtgctgactaagagcaacaaatctgact

SEQ ID NO: 33 - DNA, 60 bp, Artificial Sequence
  Synthetic
aagagcaacagtgctgtgggcctggagcaacaaatctgactaagagcaacaaatctgact

SEQ ID NO: 34 - DNA, 57 bp, Artificial Sequence
  Synthetic
aagagcaacagtgctggcctggagcaacaaatctgactaagagcaacaaatctgact

SEQ ID NO: 35 - DNA, 60 bp, Artificial Sequence
  Synthetic
aagagcaacagtgctgtgtgcctggagcaacaaatctgactaagagcaacaaatctgact

SEQ ID NO: 36 - DNA, 20 bp, Artificial Sequence
  Synthetic
gctactctctctttctggcc

SEQ ID NO: 37 - DNA, 23 bp, Artificial Sequence
  Synthetic
gctactctctctttctggcctgg

SEQ ID NO: 38 - RNA, 20 nt, Artificial Sequence
  Synthetic
gcuacucucucuuucuggcc

SEQ ID NO: 39 - RNA, 20 nt, Artificial Sequence
  Synthetic
  misc_feature (1)..(4): modified with 2′-O-methyl phosphorothioate
gcuacucucucuuucuggcc

SEQ ID NO: 40 - RNA, 100 nt, Artificial Sequence
  Synthetic
gcuacucucucuuucuggccguuuuagagcuagaaauagcaaguuaaaauaaggcuaguccguuaucaacuugaaaaaguggcaccgagucggugcuuuu

SEQ ID NO: 41 - RNA, 100 nt, Artificial Sequence; identical to SEQ ID NO: 40
  Synthetic
  misc_feature (1)..(4): modified with 2′-O-methyl phosphorothioate
  misc_feature (97)..(100): modified with 2′-O-methyl phosphorothioate

SEQ ID NO: 42 - DNA, 79 bp, Artificial Sequence
  Synthetic
cgtggccttagctgtgctcgcgctactctctctttctgcctggaggctatccagcgtgagtctctcctaccctcccgct

SEQ ID NO: 43 - DNA, 78 bp, Artificial Sequence
  Synthetic
cgtggccttagctgtgctcgcgctactctctctttcgcctggaggctatccagcgtgagtctctcctaccctcccgct

SEQ ID NO: 44 - DNA, 75 bp, Artificial Sequence
  Synthetic
cgtggccttagctgtgctcgcgctactctctctttctggaggctatccagcgtgagtctctcctaccctcccgct

SEQ ID NO: 45 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 46 - DNA, 55 bp, Artificial Sequence
  Synthetic
cgtggccttagctgtgctcgcgctatccagcgtgagtctctcctaccctcccgct

SEQ ID NO: 47 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 48 - DNA, 20 bp, Artificial Sequence
  Synthetic
ggggccactagggacaggat

SEQ ID NO: 49 - DNA, 23 bp, Artificial Sequence
  Synthetic
ggggccactagggacaggattgg

SEQ ID NO: 50 - RNA, 20 nt, Artificial Sequence
  Synthetic
ggggccacuagggacaggau

SEQ ID NO: 51 - RNA, 20 nt, Artificial Sequence
  Synthetic
  misc_feature (1)..(4): modified with 2′-O-methyl phosphorothioate
ggggccacuagggacaggau

SEQ ID NO: 52 - DNA, 100 bp, Artificial Sequence
  Synthetic
ggggccactagggacaggatguuuuagagcuagaaauagcaaguuaaaauaaggcuaguccguuaucaacuugaaaaaguggcaccgagucggugcuuuu

SEQ ID NO: 53 - DNA, 100 bp, Artificial Sequence; identical to SEQ ID NO: 52
  Synthetic
  misc_feature (1)..(4): modified with 2′-O-methyl phosphorothioate

SEQ ID NO: 54 - DNA, 20 bp, Artificial Sequence
  Synthetic
gctttggtcccattggtcgc

SEQ ID NO: 55 - DNA, 23 bp, Artificial Sequence
  Synthetic
gctttggtcccattggtcgctgg

SEQ ID NO: 56 - RNA, 20 nt, Artificial Sequence
  Synthetic
gcuuuggucccauuggucgc

SEQ ID NO: 57 - RNA, 20 nt, Artificial Sequence
  Synthetic
  misc_feature (1)..(4): modified with 2′-O-methyl phosphorothioate
gcuuuggucccauuggucgc

SEQ ID NO: 58 - RNA, 100 nt, Artificial Sequence
  Synthetic
gcuuuggucccauuggucgcguuuuagagcuagaaauagcaaguuaaaauaaggcuaguccguuaucaacuugaaaaaguggcaccgagucggugcuuuu

SEQ ID NO: 59 - RNA, 100 nt, Artificial Sequence; identical to SEQ ID NO: 58
  Synthetic
  misc_feature (1)..(4): modified with 2′-O-methyl phosphorothioate

SEQ ID NO: 60 - RNA, 100 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 61 - RNA, 96 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 62 - RNA, 114 nt, Artificial Sequence
  Synthetic
  misc_feature (1)..(17): n is a, c, g, or u
  misc_feature (18)..(30): n is a, c, g, u, or absent
  misc_feature (108)..(114): may be absent
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 63 - protein, 1368 aa, Artificial Sequence [sequence omitted]

SEQ ID NO: 64 - protein, 290 aa, Artificial Sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSLMKQIQSHGFPPEVEEQDDGTLPMSCAQESGMDRHPAACASARINV

SEQ ID NO: 65 - DNA, 13 bp, Artificial Sequence
  Synthetic
tggggattcccca

SEQ ID NO: 66 - DNA, 30 bp, Artificial Sequence
  Synthetic
ggaggaaaaactgtttcatacagaaggcgt

SEQ ID NO: 67 - DNA, 7 bp, Artificial Sequence
  Synthetic
tgactca

SEQ ID NO: 68 - DNA, 9 bp, Artificial Sequence
  Synthetic
ttctgagaa

SEQ ID NO: 69 - DNA, 8 bp, Artificial Sequence
  Synthetic
gtctagac

SEQ ID NO: 70 - DNA, 114 bp, Artificial Sequence
  Synthetic
cattttgacacccccataatatttttccagaattaacagtataaattgcatctcttgttcaagagttccctatcactctctttaatcactactcacagtaacctcaactcctgc

SEQ ID NO: 71 - DNA, 51 bp, Artificial Sequence
  Synthetic
agacgctagcggggggctataaaagggggtgggggcgttcgtcctcactct

SEQ ID NO: 72 - DNA, 700 bp, Artificial Sequence
  Synthetic
actgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 73 - DNA, 700 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcacca

SEQ ID NO: 74 - DNA, 2510 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 75 - DNA, 2600 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagttcccatcctcgaggctggggattccccatctcgaggctggggattccccatctcgaggctggggattccccatctcgaccgtccatccatcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 76 - DNA, 2666 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtcttacgcgtgctagccccgatgttttctgagttacttttgtatccccaccccccctcgaggaggaaaaactgtttcatacagaaggcgtacgccttctgtatgaaacagtttttcctccacgccttctgtatgaaacagtttttcctcctcgaggacattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 77 - DNA, 2593 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagttacgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 78 - DNA, 2611 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtgcctaactggccggtacctgagctcagttctgagaaaagtagttctgagaaaagtagttctgagaaaagtagttctgagaaaagtagttctgagaaaagtccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 79 - DNA, 2618 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtatcgataggtactaagtctagacggcagtctagacgtactaagtctagacggcagtctagacgtaccgagctcttacgcgtgctagcccgggctcgagatctgcgatccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 80 - DNA, 2447 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtagacgctagcggggggctataaaagggggtgggggcgttcgtcctcactctaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 81 - DNA, 2537 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagttcccatcctcgaggctggggattccccatctcgaggctggggattccccatctcgaggctggggattccccatctcgaccgtccatccatagacgctagcggggggctataaaagggggtgggggcgttcgtcctcactctaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 82 - DNA, 2603 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtcttacgcgtgctagccccgatgttttctgagttacttttgtatccccaccccccctcgaggaggaaaaactgtttcatacagaaggcgtacgccttctgtatgaaacagtttttcctccacgccttctgtatgaaacagtttttcctcctcgaggaagacgctagcggggggctataaaagggggtgggggcgttcgtcctcactctaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 83 - DNA, 2530 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagttacgcgtgctagcccgggcactgactcatcaagcactgactcatcaagcactgactcatcaagggactcagggagggaaactcagacgctagcggggggctataaaagggggtgggggcgttcgtcctcactctaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 84 - DNA, 2548 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtgcctaactggccggtacctgagctcagttctgagaaaagtagttctgagaaaagtagttctgagaaaagtagttctgagaaaagtagttctgagaaaagtcagacgctagcggggggctataaaagggggtgggggcgttcgtcctcactctaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 85 - DNA, 2555 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtatcgataggtactaagtctagacggcagtctagacgtactaagtctagacggcagtctagacgtaccgagctcttacgcgtgctagcccgggctcgagatctgcgatcagacgctagcggggggctataaaagggggtgggggcgttcgtcctcactctaaggccagcccagcaccagcaccagccaactctcactgaagccagctctctcttcctccaccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctctcatgaagcagatccagagccatggcttcccgccggaggtggaggagcaggatgatggcacgctgcccatgtcttgtgcccaggagagcgggatggaccgtcaccctgcagcctgtgcttctgctaggatcaatgtgtagaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 86 - DNA, 800 bp, Artificial Sequence
  Synthetic
ccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttct

SEQ ID NO: 87 - DNA, 800 bp, Artificial Sequence
  Synthetic
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagct

SEQ ID NO: 88 - DNA, 800 bp, Artificial Sequence
  Synthetic
gggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagct

SEQ ID NO: 89 - DNA, 800 bp, Artificial Sequence
  Synthetic
gttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggct

SEQ ID NO: 90 - DNA, 3261 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgtcatcagcaactggtcatctcatggttttccctggtgtttttggcgtcaccactggtggcaatatgggagcttaagaaggacgtctacgttgtcgagctggattggtacccagacgctccaggagaaatggtcgttctgacgtgtgacacacctgaggaagatggtattacctggacgcttgatcagtcatccgaagttcttgggtccgggaagacccttacaatccaggtcaaagagttcggagatgctggtcagtatacttgccacaagggcggtgaggtccttagtcacagtttgcttctgctccacaagaaggaggacggcatatggagcacagatatattgaaagaccaaaaagaacccaaaaataagacattccttcgctgcgaggccaagaactacagcggccggtttacgtgctggtggctcacaaccatatccacagatctgacgttcagtgttaaatcctcaaggggtagtagcgatccgcaaggggttacgtgcggtgctgctacccttagtgctgaaagggtcagaggggacaacaaagagtacgaatatagtgtcgaatgccaggaagatagtgcgtgtccggcggcagaagagtcactgccaattgaggtgatggtcgacgctgtgcacaaattgaaatacgagaattatacctcaagtttcttcatcagagatattataaagcctgacccgcccaaaaatttgcaactcaaaccactgaaaaatagccgccaggtggaagtctcatgggaatatcctgatacctggtccacaccccactcctatttctcactcacattttgcgttcaggtccagggaaagtccaagcgagaaaaaaaagatcgcgttttcacggacaaaacctcagccacagtgatttgccgcaagaatgcttccatatccgtacgcgctcaagacaggtattactcatcttcatggtctgaatgggcctctgtaccctgttcaggaggaggtggcagtggcgggggcggatcaggcggtggaggtagcagaaatttgccagtggcaacgccagatcctggtatgttcccgtgcctccaccactctcagaacctcttgagggctgtgtccaacatgttgcaaaaggcgcgccaaacgctcgagttttacccatgtacatcagaggaaattgaccacgaggacattacgaaggataaaaccagcacagtagaggcatgtctgccattggaactcacgaaaaacgaatcatgccttaacagccgagagacttctttcatcactaacggatcttgtcttgcctcaagaaagacttcattcatgatggccctctgcctctcctcaatctacgaagacctcaaaatgtaccaagttgagttcaagaccatgaacgctaaactccttatggatccaaagcgccaaatctttttggaccaaaacatgttggctgtgatagacgagctgatgcaggctctcaacttcaatagcgagaccgtgccccaaaagtcatcccttgaagaaccagatttttataaaacgaagattaaattgtgtattctgcttcacgctttccggatccgcgctgtgaccattgatcgagttatgtcttatctgaacgcctcttaataataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 91 - DNA, 3343 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagttacgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgtcatcagcaactggtcatctcatggttttccctggtgtttttggcgtcaccactggtggcaatatgggagcttaagaaggacgtctacgttgtcgagctggattggtacccagacgctccaggagaaatggtcgttctgacgtgtgacacacctgaggaagatggtattacctggacgcttgatcagtcatccgaagttcttgggtccgggaagacccttacaatccaggtcaaagagttcggagatgctggtcagtatacttgccacaagggcggtgaggtccttagtcacagtttgcttctgctccacaagaaggaggacggcatatggagcacagatatattgaaagaccaaaaagaacccaaaaataagacattccttcgctgcgaggccaagaactacagcggccggtttacgtgctggtggctcacaaccatatccacagatctgacgttcagtgttaaatcctcaaggggtagtagcgatccgcaaggggttacgtgcggtgctgctacccttagtgctgaaagggtcagaggggacaacaaagagtacgaatatagtgtcgaatgccaggaagatagtgcgtgtccggcggcagaagagtcactgccaattgaggtgatggtcgacgctgtgcacaaattgaaatacgagaattatacctcaagtttcttcatcagagatattataaagcctgacccgcccaaaaatttgcaactcaaaccactgaaaaatagccgccaggtggaagtctcatgggaatatcctgatacctggtccacaccccactcctatttctcactcacattttgcgttcaggtccagggaaagtccaagcgagaaaaaaaagatcgcgttttcacggacaaaacctcagccacagtgatttgccgcaagaatgcttccatatccgtacgcgctcaagacaggtattactcatcttcatggtctgaatgggcctctgtaccctgttcaggaggaggtggcagtggcgggggcggatcaggcggtggaggtagcagaaatttgccagtggcaacgccagatcctggtatgttcccgtgcctccaccactctcagaacctcttgagggctgtgtccaacatgttgcaaaaggcgcgccaaacgctcgagttttacccatgtacatcagaggaaattgaccacgaggacattacgaaggataaaaccagcacagtagaggcatgtctgccattggaactcacgaaaaacgaatcatgccttaacagccgagagacttctttcatcactaacggatcttgtcttgcctcaagaaagacttcattcatgatggccctctgcctctcctcaatctacgaagacctcaaaatgtaccaagttgagttcaagaccatgaacgctaaactccttatggatccaaagcgccaaatctttttggaccaaaacatgttggctgtgatagacgagctgatgcaggctctcaacttcaatagcgagaccgtgccccaaaagtcatcccttgaagaaccagatttttataaaacgaagattaaattgtgtattctgcttcacgctttccggatccgcgctgtgaccattgatcgagttatgtcttatctgaacgcctcttaaaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 92 - DNA, 3261 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagtcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgcccagcccggagtcttctgctggtagcaacattggttctcctggaccatttgtcactggcaagaaacctgccggtagcaacccccgatcctggtatgttcccttgtttgcatcactcacaaaaccttctccgcgccgtttctaatatgctgcaaaaggcacggcagacccttgaattttacccgtgtacatccgaagaaatcgaccatgaagacattaccaaggataagacctccacggtggaagcttgtctccctttggaacttaccaagaatgaaagctgccttaactctcgagagacttctttcatcactaatggaagctgcctggcgtcccggaaaacgtccttcatgatggcgctttgtctctcctccatctacgaggatctcaaaatgtaccaggtggaatttaagacgatgaacgcaaagcttctgatggatcccaagagacagatatttctggaccaaaacatgttggctgtcatcgacgaactcatgcaggctttgaattttaactccgagacggtgccacagaagtcctccctcgaagaaccggatttctataagactaaaattaaattgtgcatcctgttgcacgcgtttcgcattcgggccgtcacaattgacagagtaatgagttacctgaacgcctcaggtgggggtggctccggtggaggaggatcaggcggtggtggcagtatttgggaattgaaaaaggatgtctatgttgtagaacttgattggtatccggacgctccaggtgaaatggtcgttctgacgtgcgatacacctgaggaagatgggatcacatggacactcgaccagagctctgaggtcctcggtagcggcaagacgctcacaatccaggttaaggagttcggggacgcggggcagtatacttgccataagggcggggaagtgctctctcatagcctgctccttctgcacaagaaggaagatgggatatggtccacggacatccttaaagaccaaaaggagccaaagaataaaacgtttctcaggtgtgaagcgaaaaactattctgggaggtttacctgttggtggctcacgacgatctccacagacttgacattcagtgttaaatctagcaggggatcatctgacccacagggagtaacttgtggggccgcaactctctcagccgagagagtgagaggggacaataaagagtacgaatattcagtagagtgccaagaggacagcgcctgccccgctgcggaagaaagtctgccgattgaagtcatggtcgacgccgtccataagttgaagtacgaaaattacacgtcttctttttttattcgagacataataaaaccagaccccccaaaaaatctccaactgaagcccttgaaaaactcacgccaggttgaagtgagctgggaatatcccgacacctggtccacgccgcattcttattttagcttgacgttttgtgtacaggttcagggtaagagtaaacgagaaaaaaaagaccgagtttttacagacaagacttctgccacagtcatctgcagaaaaaatgcaagtatcagtgtaagagcgcaggaccgctactactcttcctcttggagcgagtgggcgtcagttccttgcagctaataataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 93 - DNA, 3343 bp, Artificial Sequence
  Synthetic
gaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccagccactagttacgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgcccagcccggagtcttctgctggtagcaacattggttctcctggaccatttgtcactggcaagaaacctgccggtagcaacccccgatcctggtatgttcccttgtttgcatcactcacaaaaccttctccgcgccgtttctaatatgctgcaaaaggcacggcagacccttgaattttacccgtgtacatccgaagaaatcgaccatgaagacattaccaaggataagacctccacggtggaagcttgtctccctttggaacttaccaagaatgaaagctgccttaactctcgagagacttctttcatcactaatggaagctgcctggcgtcccggaaaacgtccttcatgatggcgctttgtctctcctccatctacgaggatctcaaaatgtaccaggtggaatttaagacgatgaacgcaaagcttctgatggatcccaagagacagatatttctggaccaaaacatgttggctgtcatcgacgaactcatgcaggctttgaattttaactccgagacggtgccacagaagtcctccctcgaagaaccggatttctataagactaaaattaaattgtgcatcctgttgcacgcgtttcgcattcgggccgtcacaattgacagagtaatgagttacctgaacgcctcaggtgggggtggctccggtggaggaggatcaggcggtggtggcagtatttgggaattgaaaaaggatgtctatgttgtagaacttgattggtatccggacgctccaggtgaaatggtcgttctgacgtgcgatacacctgaggaagatgggatcacatggacactcgaccagagctctgaggtcctcggtagcggcaagacgctcacaatccaggttaaggagttcggggacgcggggcagtatacttgccataagggcggggaagtgctctctcatagcctgctccttctgcacaagaaggaagatgggatatggtccacggacatccttaaagaccaaaaggagccaaagaataaaacgtttctcaggtgtgaagcgaaaaactattctgggaggtttacctgttggtggctcacgacgatctccacagacttgacattcagtgttaaatctagcaggggatcatctgacccacagggagtaacttgtggggccgcaactctctcagccgagagagtgagaggggacaataaagagtacgaatattcagtagagtgccaagaggacagcgcctgccccgctgcggaagaaagtctgccgattgaagtcatggtcgacgccgtccataagttgaagtacgaaaattacacgtcttctttttttattcgagacataataaaaccagaccccccaaaaaatctccaactgaagcccttgaaaaactcacgccaggttgaagtgagctgggaatatcccgacacctggtccacgccgcattcttattttagcttgacgttttgtgtacaggttcagggtaagagtaaacgagaaaaaaaagaccgagtttttacagacaagacttctgccacagtcatctgcagaaaaaatgcaagtatcagtgtaagagcgcaggaccgctactactcttcctcttggagcgagtgggcgtcagttccttgcagctaaaataaaatcgctatccatcgaagatggatgtgtgttggttttttgtgtgactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctccggaaagagcatccttgggcagcaacacagcagagagcaaggggaagagggagtggaggaagacggaacctgaaggaggcggc

SEQ ID NO: 94 - DNA, 3452 bp, Artificial Sequence
  Synthetic
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagcttcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgtcatcagcaactggtcatctcatggttttccctggtgtttttggcgtcaccactggtggcaatatgggagcttaagaaggacgtctacgttgtcgagctggattggtacccagacgctccaggagaaatggtcgttctgacgtgtgacacacctgaggaagatggtattacctggacgcttgatcagtcatccgaagttcttgggtccgggaagacccttacaatccaggtcaaagagttcggagatgctggtcagtatacttgccacaagggcggtgaggtccttagtcacagtttgcttctgctccacaagaaggaggacggcatatggagcacagatatattgaaagaccaaaaagaacccaaaaataagacattccttcgctgcgaggccaagaactacagcggccggtttacgtgctggtggctcacaaccatatccacagatctgacgttcagtgttaaatcctcaaggggtagtagcgatccgcaaggggttacgtgcggtgctgctacccttagtgctgaaagggtcagaggggacaacaaagagtacgaatatagtgtcgaatgccaggaagatagtgcgtgtccggcggcagaagagtcactgccaattgaggtgatggtcgacgctgtgcacaaattgaaatacgagaattatacctcaagtttcttcatcagagatattataaagcctgacccgcccaaaaatttgcaactcaaaccactgaaaaatagccgccaggtggaagtctcatgggaatatcctgatacctggtccacaccccactcctatttctcactcacattttgcgttcaggtccagggaaagtccaagcgagaaaaaaaagatcgcgttttcacggacaaaacctcagccacagtgatttgccgcaagaatgcttccatatccgtacgcgctcaagacaggtattactcatcttcatggtctgaatgggcctctgtaccctgttcaggaggaggtggcagtggcgggggcggatcaggcggtggaggtagcagaaatttgccagtggcaacgccagatcctggtatgttcccgtgcctccaccactctcagaacctcttgagggctgtgtccaacatgttgcaaaaggcgcgccaaacgctcgagttttacccatgtacatcagaggaaattgaccacgaggacattacgaaggataaaaccagcacagtagaggcatgtctgccattggaactcacgaaaaacgaatcatgccttaacagccgagagacttctttcatcactaacggatcttgtcttgcctcaagaaagacttcattcatgatggccctctgcctctcctcaatctacgaagacctcaaaatgtaccaagttgagttcaagaccatgaacgctaaactccttatggatccaaagcgccaaatctttttggaccaaaacatgttggctgtgatagacgagctgatgcaggctctcaacttcaatagcgagaccgtgccccaaaagtcatcccttgaagaaccagatttttataaaacgaagattaaattgtgtattctgcttcacgctttccggatccgcgctgtgaccattgatcgagttatgtcttatctgaacgcctcttaataataaaatcgctatccatcgaagatggatgtgtgttggttttttgtgtgccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttc

SEQ ID NO: 95 - DNA, 3535 bp, Artificial Sequence
  Synthetic
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagcttacgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgtcatcagcaactggtcatctcatggttttccctggtgtttttggcgtcaccactggtggcaatatgggagcttaagaaggacgtctacgttgtcgagctggattggtacccagacgctccaggagaaatggtcgttctgacgtgtgacacacctgaggaagatggtattacctggacgcttgatcagtcatccgaagttcttgggtccgggaagacccttacaatccaggtcaaagagttcggagatgctggtcagtatacttgccacaagggcggtgaggtccttagtcacagtttgcttctgctccacaagaaggaggacggcatatggagcacagatatattgaaagaccaaaaagaacccaaaaataagacattccttcgctgcgaggccaagaactacagcggccggtttacgtgctggtggctcacaaccatatccacagatctgacgttcagtgttaaatcctcaaggggtagtagcgatccgcaaggggttacgtgcggtgctgctacccttagtgctgaaagggtcagaggggacaacaaagagtacgaatatagtgtcgaatgccaggaagatagtgcgtgtccggcggcagaagagtcactgccaattgaggtgatggtcgacgctgtgcacaaattgaaatacgagaattatacctcaagtttcttcatcagagatattataaagcctgacccgcccaaaaatttgcaactcaaaccactgaaaaatagccgccaggtggaagtctcatgggaatatcctgatacctggtccacaccccactcctatttctcactcacattttgcgttcaggtccagggaaagtccaagcgagaaaaaaaagatcgcgttttcacggacaaaacctcagccacagtgatttgccgcaagaatgcttccatatccgtacgcgctcaagacaggtattactcatcttcatggtctgaatgggcctctgtaccctgttcaggaggaggtggcagtggcgggggcggatcaggcggtggaggtagcagaaatttgccagtggcaacgccagatcctggtatgttcccgtgcctccaccactctcagaacctcttgagggctgtgtccaacatgttgcaaaaggcgcgccaaacgctcgagttttacccatgtacatcagaggaaattgaccacgaggacattacgaaggataaaaccagcacagtagaggcatgtctgccattggaactcacgaaaaacgaatcatgccttaacagccgagagacttctttcatcactaacggatcttgtcttgcctcaagaaagacttcattcatgatggccctctgcctctcctcaatctacgaagacctcaaaatgtaccaagttgagttcaagaccatgaacgctaaactccttatggatccaaagcgccaaatctttttggaccaaaacatgttggctgtgatagacgagctgatgcaggctctcaacttcaatagcgagaccgtgccccaaaagtcatcccttgaagaaccagatttttataaaacgaagattaaattgtgtattctgcttcacgctttccggatccgcgctgtgaccattgatcgagttatgtcttatctgaacgcctcttaataataaaatcgctatccatcgaagatggatgtgtgttggttttttgtgtgccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttct

SEQ ID NO: 96 - DNA, 3453 bp, Artificial Sequence
  Synthetic
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagcttcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgcccagcccggagtcttctgctggtagcaacattggttctcctggaccatttgtcactggcaagaaacctgccggtagcaacccccgatcctggtatgttcccttgtttgcatcactcacaaaaccttctccgcgccgtttctaatatgctgcaaaaggcacggcagacccttgaattttacccgtgtacatccgaagaaatcgaccatgaagacattaccaaggataagacctccacggtggaagcttgtctccctttggaacttaccaagaatgaaagctgccttaactctcgagagacttctttcatcactaatggaagctgcctggcgtcccggaaaacgtccttcatgatggcgctttgtctctcctccatctacgaggatctcaaaatgtaccaggtggaatttaagacgatgaacgcaaagcttctgatggatcccaagagacagatatttctggaccaaaacatgttggctgtcatcgacgaactcatgcaggctttgaattttaactccgagacggtgccacagaagtcctccctcgaagaaccggatttctataagactaaaattaaattgtgcatcctgttgcacgcgtttcgcattcgggccgtcacaattgacagagtaatgagttacctgaacgcctcaggtgggggtggctccggtggaggaggatcaggcggtggtggcagtatttgggaattgaaaaaggatgtctatgttgtagaacttgattggtatccggacgctccaggtgaaatggtcgttctgacgtgcgatacacctgaggaagatgggatcacatggacactcgaccagagctctgaggtcctcggtagcggcaagacgctcacaatccaggttaaggagttcggggacgcggggcagtatacttgccataagggcggggaagtgctctctcatagcctgctccttctgcacaagaaggaagatgggatatggtccacggacatccttaaagaccaaaaggagccaaagaataaaacgtttctcaggtgtgaagcgaaaaactattctgggaggtttacctgttggtggctcacgacgatctccacagacttgacattcagtgttaaatctagcaggggatcatctgacccacagggagtaacttgtggggccgcaactctctcagccgagagagtgagaggggacaataaagagtacgaatattcagtagagtgccaagaggacagcgcctgccccgctgcggaagaaagtctgccgattgaagtcatggtcgacgccgtccataagttgaagtacgaaaattacacgtcttctttttttattcgagacataataaaaccagaccccccaaaaaatctccaactgaagcccttgaaaaactcacgccaggttgaagtgagctgggaatatcccgacacctggtccacgccgcattcttattttagcttgacgttttgtgtacaggttcagggtaagagtaaacgagaaaaaaaagaccgagtttttacagacaagacttctgccacagtcatctgcagaaaaaatgcaagtatcagtgtaagagcgcaggaccgctactactcttcctcttggagcgagtgggcgtcagttccttgcagctaataataaaatcgctatccatcgaagatggatgtgtgttggttttttgtgtgccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttct

SEQ ID NO: 97 - DNA, 3535 bp, Artificial Sequence
  Synthetic
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagcttacgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgcccagcccggagtcttctgctggtagcaacattggttctcctggaccatttgtcactggcaagaaacctgccggtagcaacccccgatcctggtatgttcccttgtttgcatcactcacaaaaccttctccgcgccgtttctaatatgctgcaaaaggcacggcagacccttgaattttacccgtgtacatccgaagaaatcgaccatgaagacattaccaaggataagacctccacggtggaagcttgtctccctttggaacttaccaagaatgaaagctgccttaactctcgagagacttctttcatcactaatggaagctgcctggcgtcccggaaaacgtccttcatgatggcgctttgtctctcctccatctacgaggatctcaaaatgtaccaggtggaatttaagacgatgaacgcaaagcttctgatggatcccaagagacagatatttctggaccaaaacatgttggctgtcatcgacgaactcatgcaggctttgaattttaactccgagacggtgccacagaagtcctccctcgaagaaccggatttctataagactaaaattaaattgtgcatcctgttgcacgcgtttcgcattcgggccgtcacaattgacagagtaatgagttacctgaacgcctcaggtgggggtggctccggtggaggaggatcaggcggtggtggcagtatttgggaattgaaaaaggatgtctatgttgtagaacttgattggtatccggacgctccaggtgaaatggtcgttctgacgtgcgatacacctgaggaagatgggatcacatggacactcgaccagagctctgaggtcctcggtagcggcaagacgctcacaatccaggttaaggagttcggggacgcggggcagtatacttgccataagggcggggaagtgctctctcatagcctgctccttctgcacaagaaggaagatgggatatggtccacggacatccttaaagaccaaaaggagccaaagaataaaacgtttctcaggtgtgaagcgaaaaactattctgggaggtttacctgttggtggctcacgacgatctccacagacttgacattcagtgttaaatctagcaggggatcatctgacccacagggagtaacttgtggggccgcaactctctcagccgagagagtgagaggggacaataaagagtacgaatattcagtagagtgccaagaggacagcgcctgccccgctgcggaagaaagtctgccgattgaagtcatggtcgacgccgtccataagttgaagtacgaaaattacacgtcttctttttttattcgagacataataaaaccagaccccccaaaaaatctccaactgaagcccttgaaaaactcacgccaggttgaagtgagctgggaatatcccgacacctggtccacgccgcattcttattttagcttgacgttttgtgtacaggttcagggtaagagtaaacgagaaaaaaaagaccgagtttttacagacaagacttctgccacagtcatctgcagaaaaaatgcaagtatcagtgtaagagcgcaggaccgctactactcttcctcttggagcgagtgggcgtcagttccttgcagctaataataaaatcgctatccatcgaagatggatgtgtgttggttttttgtgtgccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttct

SEQ ID NO: 98 - DNA, 3452 bp, Artificial Sequence
  Synthetic
gttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgtcatcagcaactggtcatctcatggttttccctggtgtttttggcgtcaccactggtggcaatatgggagcttaagaaggacgtctacgttgtcgagctggattggtacccagacgctccaggagaaatggtcgttctgacgtgtgacacacctgaggaagatggtattacctggacgcttgatcagtcatccgaagttcttgggtccgggaagacccttacaatccaggtcaaagagttcggagatgctggtcagtatacttgccacaagggcggtgaggtccttagtcacagtttgcttctgctccacaagaaggaggacggcatatggagcacagatatattgaaagaccaaaaagaacccaaaaataagacattccttcgctgcgaggccaagaactacagcggccggtttacgtgctggtggctcacaaccatatccacagatctgacgttcagtgttaaatcctcaaggggtagtagcgatccgcaaggggttacgtgcggtgctgctacccttagtgctgaaagggtcagaggggacaacaaagagtacgaatatagtgtcgaatgccaggaagatagtgcgtgtccggcggcagaagagtcactgccaattgaggtgatggtcgacgctgtgcacaaattgaaatacgagaattatacctcaagtttcttcatcagagatattataaagcctgacccgcccaaaaatttgcaactcaaaccactgaaaaatagccgccaggtggaagtctcatgggaatatcctgatacctggtccacaccccactcctatttctcactcacattttgcgttcaggtccagggaaagtccaagcgagaaaaaaaagatcgcgttttcacggacaaaacctcagccacagtgatttgccgcaagaatgcttccatatccgtacgcgctcaagacaggtattactcatcttcatggtctgaatgggcctctgtaccctgttcaggaggaggtggcagtggcgggggcggatcaggcggtggaggtagcagaaatttgccagtggcaacgccagatcctggtatgttcccgtgcctccaccactctcagaacctcttgagggctgtgtccaacatgttgcaaaaggcgcgccaaacgctcgagttttacccatgtacatcagaggaaattgaccacgaggacattacgaaggataaaaccagcacagtagaggcatgtctgccattggaactcacgaaaaacgaatcatgccttaacagccgagagacttctttcatcactaacggatcttgtcttgcctcaagaaagacttcattcatgatggccctctgcctctcctcaatctacgaagacctcaaaatgtaccaagttgagttcaagaccatgaacgctaaactccttatggatccaaagcgccaaatctttttggaccaaaacatgttggctgtgatagacgagctgatgcaggctctcaacttcaatagcgagaccgtgccccaaaagtcatcccttgaagaaccagatttttataaaacgaagattaaattgtgtattctgcttcacgctttccggatccgcgctgtgaccattgatcgagttatgtcttatctgaacgcctcttaataataaaatcgctatccatcgaagatggatgtgtgttggttttttgtgtggggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagct

SEQ ID NO: 99 - DNA, 3534 bp, Artificial Sequence
  Synthetic
gttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctacgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgtcatcagcaactggtcatctcatggttttccctggtgtttttggcgtcaccactggtggcaatatgggagcttaagaaggacgtctacgttgtcgagctggattggtacccagacgctccaggagaaatggtcgttctgacgtgtgacacacctgaggaagatggtattacctggacgcttgatcagtcatccgaagttcttgggtccgggaagacccttacaatccaggtcaaagagttcggagatgctggtcagtatacttgccacaagggcggtgaggtccttagtcacagtttgcttctgctccacaagaaggaggacggcatatggagcacagatatattgaaagaccaaaaagaacccaaaaataagacattccttcgctgcgaggccaagaactacagcggccggtttacgtgctggtggctcacaaccatatccacagatctgacgttcagtgttaaatcctcaaggggtagtagcgatccgcaaggggttacgtgcggtgctgctacccttagtgctgaaagggtcagaggggacaacaaagagtacgaatatagtgtcgaatgccaggaagatagtgcgtgtccggcggcagaagagtcactgccaattgaggtgatggtcgacgctgtgcacaaattgaaatacgagaattatacctcaagtttcttcatcagagatattataaagcctgacccgcccaaaaatttgcaactcaaaccactgaaaaatagccgccaggtggaagtctcatgggaatatcctgatacctggtccacaccccactcctatttctcactcacattttgcgttcaggtccagggaaagtccaagcgagaaaaaaaagatcgcgttttcacggacaaaacctcagccacagtgatttgccgcaagaatgcttccatatccgtacgcgctcaagacaggtattactcatcttcatggtctgaatgggcctctgtaccctgttcaggaggaggtggcagtggcgggggcggatcaggcggtggaggtagcagaaatttgccagtggcaacgccagatcctggtatgttcccgtgcctccaccactctcagaacctcttgagggctgtgtccaacatgttgcaaaaggcgcgccaaacgctcgagttttacccatgtacatcagaggaaattgaccacgaggacattacgaaggataaaaccagcacagtagaggcatgtctgccattggaactcacgaaaaacgaatcatgccttaacagccgagagacttctttcatcactaacggatcttgtcttgcctcaagaaagacttcattcatgatggccctctgcctctcctcaatctacgaagacctcaaaatgtaccaagttgagttcaagaccatgaacgctaaactccttatggatccaaagcgccaaatctttttggaccaaaacatgttggctgtgatagacgagctgatgcaggctctcaacttcaatagcgagaccgtgccccaaaagtcatcccttgaagaaccagatttttataaaacgaagattaaattgtgtattctgcttcacgctttccggatccgcgctgtgaccattgatcgagttatgtcttatctgaacgcctcttaataataaaatcgctatccatcgaagatggatgtgtgttggttttttgtgtggggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagct

SEQ ID NO: 100 - DNA, 3452 bp, Artificial Sequence
  Synthetic
gttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctcattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgcccagcccggagtcttctgctggtagcaacattggttctcctggaccatttgtcactggcaagaaacctgccggtagcaacccccgatcctggtatgttcccttgtttgcatcactcacaaaaccttctccgcgccgtttctaatatgctgcaaaaggcacggcagacccttgaattttacccgtgtacatccgaagaaatcgaccatgaagacattaccaaggataagacctccacggtggaagcttgtctccctttggaacttaccaagaatgaaagctgccttaactctcgagagacttctttcatcactaatggaagctgcctggcgtcccggaaaacgtccttcatgatggcgctttgtctctcctccatctacgaggatctcaaaatgtaccaggtggaatttaagacgatgaacgcaaagcttctgatggatcccaagagacagatatttctggaccaaaacatgttggctgtcatcgacgaactcatgcaggctttgaattttaactccgagacggtgccacagaagtcctccctcgaagaaccggatttctataagactaaaattaaattgtgcatcctgttgcacgcgtttcgcattcgggccgtcacaattgacagagtaatgagttacctgaacgcctcaggtgggggtggctccggtggaggaggatcaggcggtggtggcagtatttgggaattgaaaaaggatgtctatgttgtagaacttgattggtatccggacgctccaggtgaaatggtcgttctgacgtgcgatacacctgaggaagatgggatcacatggacactcgaccagagctctgaggtcctcggtagcggcaagacgctcacaatccaggttaaggagttcggggacgcggggcagtatacttgccataagggcggggaagtgctctctcatagcctgctccttctgcacaagaaggaagatgggatatggtccacggacatccttaaagaccaaaaggagccaaagaataaaacgtttctcaggtgtgaagcgaaaaactattctgggaggtttacctgttggtggctcacgacgatctccacagacttgacattcagtgttaaatctagcaggggatcatctgacccacagggagtaacttgtggggccgcaactctctcagccgagagagtgagaggggacaataaagagtacgaatattcagtagagtgccaagaggacagcgcctgccccgctgcggaagaaagtctgccgattgaagtcatggtcgacgccgtccataagttgaagtacgaaaattacacgtcttctttttttattcgagacataataaaaccagaccccccaaaaaatctccaactgaagcccttgaaaaactcacgccaggttgaagtgagctgggaatatcccgacacctggtccacgccgcattcttattttagcttgacgttttgtgtacaggttcagggtaagagtaaacgagaaaaaaaagaccgagtttttacagacaagacttctgccacagtcatctgcagaaaaaatgcaagtatcagtgtaagagcgcaggaccgctactactcttcctcttggagcgagtgggcgtcagttccttgcagctaataataaaatcgctatccatcgaagatggatgtgtgttggttttttgtgtggggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagct

SEQ ID NO: 101 - DNA, 3534 bp, Artificial Sequence
  Synthetic
gttcaagtccagcctggccaacatggtgaaaccccatctctactaaaaatacaaaaaattagccaggcatggtggcgcgcgcatgttactcccagctactcgcgaggctcagacaggagaatcgcttgaacccaggagatcgaggttgcggcgagctgagatggcgccactgcactccagcctgggtgacagagggagacctccgtctcaaaaacaaaacaaatcaaaaaaatgcaggagaggggtacacgaatatttggggagcacccccaattcttggatgtctgctgtatccccagtgcacagcacaatctaatccctaataaatgtgcagtggaggtttgttgaataaatgaatgggccccagaagaatgaggtggagaggggaataggaagattgaatgtctcctgcctgaaggtcgggcggggaggggttgggggcaggcaactctgaggctcacccggggccactgcctgcatcctggcaactgcctccacccactttaggatcttcagactggcagcggttggagggaatttcccctcgccaattgctcaagtccctcccctcgaccggccggacatccccagagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctacgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgcccagcccggagtcttctgctggtagcaacattggttctcctggaccatttgtcactggcaagaaacctgccggtagcaacccccgatcctggtatgttcccttgtttgcatcactcacaaaaccttctccgcgccgtttctaatatgctgcaaaaggcacggcagacccttgaattttacccgtgtacatccgaagaaatcgaccatgaagacattaccaaggataagacctccacggtggaagcttgtctccctttggaacttaccaagaatgaaagctgccttaactctcgagagacttctttcatcactaatggaagctgcctggcgtcccggaaaacgtccttcatgatggcgctttgtctctcctccatctacgaggatctcaaaatgtaccaggtggaatttaagacgatgaacgcaaagcttctgatggatcccaagagacagatatttctggaccaaaacatgttggctgtcatcgacgaactcatgcaggctttgaattttaactccgagacggtgccacagaagtcctccctcgaagaaccggatttctataagactaaaattaaattgtgcatcctgttgcacgcgtttcgcattcgggccgtcacaattgacagagtaatgagttacctgaacgcctcaggtgggggtggctccggtggaggaggatcaggcggtggtggcagtatttgggaattgaaaaaggatgtctatgttgtagaacttgattggtatccggacgctccaggtgaaatggtcgttctgacgtgcgatacacctgaggaagatgggatcacatggacactcgaccagagctctgaggtcctcggtagcggcaagacgctcacaatccaggttaaggagttcggggacgcggggcagtatacttgccataagggcggggaagtgctctctcatagcctgctccttctgcacaagaaggaagatgggatatggtccacggacatccttaaagaccaaaaggagccaaagaataaaacgtttctcaggtgtgaagcgaaaaactattctgggaggtttacctgttggtggctcacgacgatctccacagacttgacattcagtgttaaatctagcaggggatcatctgacccacagggagtaacttgtggggccgcaactctctcagccgagagagtgagaggggacaataaagagtacgaatattcagtagagtgccaagaggacagcgcctgccccgctgcggaagaaagtctgccgattgaagtcatggtcgacgccgtccataagttgaagtacgaaaattacacgtcttctttttttattcgagacataataaaaccagaccccccaaaaaatctccaactgaagcccttgaaaaactcacgccaggttgaagtgagctgggaatatcccgacacctggtccacgccgcattcttattttagcttgacgttttgtgtacaggttcagggtaagagtaaacgagaaaaaaaagaccgagtttttacagacaagacttctgccacagtcatctgcagaaaaaatgcaagtatcagtgtaagagcgcaggaccgctactactcttcctcttggagcgagtgggcgtcagttccttgcagctaataataaaatcgctatccatcgaagatggatgtgtgttggttttttgtgtggggcttggtgatctgcctcgtggtgtgcatccagcgcttcgcacaggctcagcagcagctgccgctcgagtcacttggggtgagttgagatggaaaagttgggaagaaaacatagagaggcgcgtgaccgaaaagacagaatgagatgggtacaaagaggccagagaggaagatctggtagggcagagacagagaccagaacagggaggcgaggcggggaccaggctgcccggtgtaggggctacgagacaggcagccctgccaggaggtacagggagatcccgggatgggaaaggtaggcacacatggaaatggaagatgactcggctctggtgttcccccggcaggctgactcagaggctgctgggggcttcacaaggctgggcgtgggggcttcctggggcctcctaggacgggatggccccagccactcgctccgggtgggggaggggtccctttggggaccgcgccgggcgcctttgcagcgtagagagtccgctgcgcgcggtgctctcgcgcccagtgacatccaggaaaacgattcgggaaacgaagaagttcttttgaaggtctcgacttcacgttccccgctggttcagacctgcttcctctttaagaagtcttaagagtaaaaaaaaataaaatgaaataaaatcaccagtgcgcgccgtgggatgagaggtggaaaggaggatggacagagaaaagagagctcctggcacaggggacacatagaacctctctgcttacgtccgtgccctgttttctggtcttttcttccagtgggacgtagctgagct

SEQ ID NO: 102 - DNA, 1934 bp, Artificial Sequence
  Synthetic
acgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgcccagcccggagtcttctgctggtagcaacattggttctcctggaccatttgtcactggcaagaaacctgccggtagcaacccccgatcctggtatgttcccttgtttgcatcactcacaaaaccttctccgcgccgtttctaatatgctgcaaaaggcacggcagacccttgaattttacccgtgtacatccgaagaaatcgaccatgaagacattaccaaggataagacctccacggtggaagcttgtctccctttggaacttaccaagaatgaaagctgccttaactctcgagagacttctttcatcactaatggaagctgcctggcgtcccggaaaacgtccttcatgatggcgctttgtctctcctccatctacgaggatctcaaaatgtaccaggtggaatttaagacgatgaacgcaaagcttctgatggatcccaagagacagatatttctggaccaaaacatgttggctgtcatcgacgaactcatgcaggctttgaattttaactccgagacggtgccacagaagtcctccctcgaagaaccggatttctataagactaaaattaaattgtgcatcctgttgcacgcgtttcgcattcgggccgtcacaattgacagagtaatgagttacctgaacgcctcaggtgggggtggctccggtggaggaggatcaggcggtggtggcagtatttgggaattgaaaaaggatgtctatgttgtagaacttgattggtatccggacgctccaggtgaaatggtcgttctgacgtgcgatacacctgaggaagatgggatcacatggacactcgaccagagctctgaggtcctcggtagcggcaagacgctcacaatccaggttaaggagttcggggacgcggggcagtatacttgccataagggcggggaagtgctctctcatagcctgctccttctgcacaagaaggaagatgggatatggtccacggacatccttaaagaccaaaaggagccaaagaataaaacgtttctcaggtgtgaagcgaaaaactattctgggaggtttacctgttggtggctcacgacgatctccacagacttgacattcagtgttaaatctagcaggggatcatctgacccacagggagtaacttgtggggccgcaactctctcagccgagagagtgagaggggacaataaagagtacgaatattcagtagagtgccaagaggacagcgcctgccccgctgcggaagaaagtctgccgattgaagtcatggtcgacgccgtccataagttgaagtacgaaaattacacgtcttctttttttattcgagacataataaaaccagaccccccaaaaaatctccaactgaagcccttgaaaaactcacgccaggttgaagtgagctgggaatatcccgacacctggtccacgccgcattcttattttagcttgacgttttgtgtacaggttcagggtaagagtaaacgagaaaaaaaagaccgagtttttacagacaagacttctgccacagtcatctgcagaaaaaatgcaagtatcagtgtaagagcgcaggaccgctactactcttcctcttggagcgagtgggcgtcagttccttgcagctaataataaaatcgctatccatcgaagatggatgtgtgttggttttttgtgtg

SEQ ID NO: 103 - DNA, 1934 bp, Artificial Sequence
  Synthetic
acgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgcaaggccagcccagcaccagcaccagccaactctcactgaagccagctctctcttcctccaccaccatgtgtcatcagcaactggtcatctcatggttttccctggtgtttttggcgtcaccactggtggcaatatgggagcttaagaaggacgtctacgttgtcgagctggattggtacccagacgctccaggagaaatggtcgttctgacgtgtgacacacctgaggaagatggtattacctggacgcttgatcagtcatccgaagttcttgggtccgggaagacccttacaatccaggtcaaagagttcggagatgctggtcagtatacttgccacaagggcggtgaggtccttagtcacagtttgcttctgctccacaagaaggaggacggcatatggagcacagatatattgaaagaccaaaaagaacccaaaaataagacattccttcgctgcgaggccaagaactacagcggccggtttacgtgctggtggctcacaaccatatccacagatctgacgttcagtgttaaatcctcaaggggtagtagcgatccgcaaggggttacgtgcggtgctgctacccttagtgctgaaagggtcagaggggacaacaaagagtacgaatatagtgtcgaatgccaggaagatagtgcgtgtccggcggcagaagagtcactgccaattgaggtgatggtcgacgctgtgcacaaattgaaatacgagaattatacctcaagtttcttcatcagagatattataaagcctgacccgcccaaaaatttgcaactcaaaccactgaaaaatagccgccaggtggaagtctcatgggaatatcctgatacctggtccacaccccactcctatttctcactcacattttgcgttcaggtccagggaaagtccaagcgagaaaaaaaagatcgcgttttcacggacaaaacctcagccacagtgatttgccgcaagaatgcttccatatccgtacgcgctcaagacaggtattactcatcttcatggtctgaatgggcctctgtaccctgttcaggaggaggtggcagtggcgggggcggatcaggcggtggaggtagcagaaatttgccagtggcaacgccagatcctggtatgttcccgtgcctccaccactctcagaacctcttgagggctgtgtccaacatgttgcaaaaggcgcgccaaacgctcgagttttacccatgtacatcagaggaaattgaccacgaggacattacgaaggataaaaccagcacagtagaggcatgtctgccattggaactcacgaaaaacgaatcatgccttaacagccgagagacttctttcatcactaacggatcttgtcttgcctcaagaaagacttcattcatgatggccctctgcctctcctcaatctacgaagacctcaaaatgtaccaagttgagttcaagaccatgaacgctaaactccttatggatccaaagcgccaaatctttttggaccaaaacatgttggctgtgatagacgagctgatgcaggctctcaacttcaatagcgagaccgtgccccaaaagtcatcccttgaagaaccagatttttataaaacgaagattaaattgtgtattctgcttcacgctttccggatccgcgctgtgaccattgatcgagttatgtcttatctgaacgcctcttaataataaaatcgctatccatcgaagatggatgtgtgttggttttttgtgtg

SEQ ID NO: 104 - DNA, 196 bp, Artificial Sequence
  Synthetic
acgcgtgctagcccgggcactgactcatcaagcactgactcatcaagcactgactcatcaagggactcagggagggaaactccattttgacacccccataatatttttccagaattaacagtataaattgcatctcttgttcaagagttccctatcactctctttaatcactactcacagtaacctcaactcctgc

SEQ ID NO: 105 - DNA, 65 bp, Artificial Sequence
  Synthetic
aaggccagcccagcaccagcaccagccaactctcactgaagccagctctctcttcctccaccacc